SEQUENCE LISTING (347 sequences)

SEQ ID NO: 1 - DNA, 20 bp, Artificial Sequence
  Description of Artificial Sequence Synthetic oligonucleotide
aagaacttccaaccggtaat

SEQ ID NO: 2 - DNA, 20 bp, Artificial Sequence
  Description of Artificial Sequence Synthetic oligonucleotide
gcaatatggaagatattcgt

SEQ ID NO: 3 - DNA, 20 bp, Artificial Sequence
  Description of Artificial Sequence Synthetic oligonucleotide
ataatatctaaatcttcatt

SEQ ID NO: 4 - DNA, 20 bp, Artificial Sequence
  Description of Artificial Sequence Synthetic oligonucleotide
atgttctgggctgttcttta

SEQ ID NO: 5 - DNA, 20 bp, Artificial Sequence
  Description of Artificial Sequence Synthetic oligonucleotide
gagcagtatcgattagcttt

SEQ ID NO: 6 - DNA, 20 bp, Artificial Sequence
  Description of Artificial Sequence Synthetic oligonucleotide
gatttcagaaggaacttcaa

SEQ ID NO: 7 - DNA, 20 bp, Artificial Sequence
  Description of Artificial Sequence Synthetic oligonucleotide
gttgtatcttatcaagtctt

SEQ ID NO: 8 - DNA, 20 bp, Artificial Sequence
  Description of Artificial Sequence Synthetic oligonucleotide
ttctgaagaagatgcttgaa

SEQ ID NO: 9 - DNA, 20 bp, Artificial Sequence
  Description of Artificial Sequence Synthetic oligonucleotide
cttccaaagggaactttaga

SEQ ID NO: 10 - DNA, 20 bp, Artificial Sequence
  Description of Artificial Sequence Synthetic oligonucleotide
tcacgaaatacaccataaat

SEQ ID NO: 11 - DNA, 20 bp, Artificial Sequence
  Description of Artificial Sequence Synthetic oligonucleotide
gaacatatcaaaaagcgtag

SEQ ID NO: 12 - DNA, 20 bp, Artificial Sequence
  Description of Artificial Sequence Synthetic oligonucleotide
ttacagaagaaattggtgat

SEQ ID NO: 13 - DNA, 20 bp, Artificial Sequence
  Description of Artificial Sequence Synthetic oligonucleotide
gcgctgcaaccaatagtctt

SEQ ID NO: 14 - DNA, 20 bp, Artificial Sequence
  Description of Artificial Sequence Synthetic oligonucleotide
gcgctttatgtttggtaaat

SEQ ID NO: 15 - DNA, 20 bp, Artificial Sequence
  Description of Artificial Sequence Synthetic oligonucleotide
gattgagttgctattcgttg

SEQ ID NO: 16 - DNA, 20 bp, Artificial Sequence
  Description of Artificial Sequence Synthetic oligonucleotide
gttagtaaatcctgccacac

SEQ ID NO: 17 - DNA, 20 bp, Artificial Sequence
  Description of Artificial Sequence Synthetic oligonucleotide
tattcgcaattggataaatg

SEQ ID NO: 18 - DNA, 20 bp, Artificial Sequence
  Description of Artificial Sequence Synthetic oligonucleotide
agcggataaggatgttttaa

SEQ ID NO: 19 - DNA, 20 bp, Artificial Sequence
  Description of Artificial Sequence Synthetic oligonucleotide
gaatagaaggcataccgctc

SEQ ID NO: 20 - DNA, 20 bp, Artificial Sequence
  Description of Artificial Sequence Synthetic oligonucleotide
tgatacctcgttgcagtatt

SEQ ID NO: 21 - DNA, 20 bp, Artificial Sequence
  Description of Artificial Sequence Synthetic oligonucleotide
aatttcgagctggtcttcgg

SEQ ID NO: 22 - DNA, 20 bp, Artificial Sequence
  Description of Artificial Sequence Synthetic oligonucleotide
tttcttccgcattcattcca

SEQ ID NO: 23 - DNA, 20 bp, Artificial Sequence
  Description of Artificial Sequence Synthetic oligonucleotide
caacgtttagaactggcact

SEQ ID NO: 24 - DNA, 20 bp, Artificial Sequence
  Description of Artificial Sequence Synthetic oligonucleotide
ggcctttactacagaagctt

SEQ ID NO: 25 - DNA, 20 bp, Artificial Sequence
  Description of Artificial Sequence Synthetic oligonucleotide
accacgggctgcattagcaa

SEQ ID NO: 26 - protein, 8 aa, Unknown
  Description of Unknown T4 phage sequence
TEDYWLQR

SEQ ID NO: 27 - protein, 8 aa, Unknown
  Description of Unknown T4 phage sequence
GNMEDIRW

SEQ ID NO: 28 - protein, 8 aa, Unknown
  Description of Unknown T4 phage sequence
QSGMPSIL

SEQ ID NO: 29 - protein, 8 aa, Unknown
  Description of Unknown T4 phage sequence
KNFQPVMG

SEQ ID NO: 30 - DNA, 58 bp, Unknown
  Description of Unknown T4 phage sequence
cgctgcgtggttccgatccgaagaacttccaaccggtaatgggattcaaaactcgtta

SEQ ID NO: 31 - DNA, 58 bp, Artificial Sequence
  Description of Artificial Sequence Synthetic oligonucleotide
cgctgcgtggttccgatccgaagaacttccaaccggtaatggtattcaaaactcgtta

SEQ ID NO: 32 - DNA, 58 bp, Artificial Sequence
  Description of Artificial Sequence Synthetic oligonucleotide
cgctgcgtggttccgatccgaagaacttccaaccggtaatggcattcaaaactcgtta

SEQ ID NO: 33 - DNA, 58 bp, Artificial Sequence; identical to SEQ ID NO: 32
  Description of Artificial Sequence Synthetic oligonucleotide

SEQ ID NO: 34 - DNA, 58 bp, Artificial Sequence; identical to SEQ ID NO: 31
  Description of Artificial Sequence Synthetic oligonucleotide

SEQ ID NO: 35 - DNA, 58 bp, Artificial Sequence; identical to SEQ ID NO: 32
  Description of Artificial Sequence Synthetic oligonucleotide

SEQ ID NO: 36 - DNA, 58 bp, Artificial Sequence; identical to SEQ ID NO: 31
  Description of Artificial Sequence Synthetic oligonucleotide

SEQ ID NO: 37 - DNA, 58 bp, Artificial Sequence; identical to SEQ ID NO: 32
  Description of Artificial Sequence Synthetic oligonucleotide

SEQ ID NO: 38 - DNA, 58 bp, Artificial Sequence; identical to SEQ ID NO: 32
  Description of Artificial Sequence Synthetic oligonucleotide

SEQ ID NO: 39 - DNA, 58 bp, Artificial Sequence; identical to SEQ ID NO: 32
  Description of Artificial Sequence Synthetic oligonucleotide

SEQ ID NO: 40 - DNA, 58 bp, Artificial Sequence; identical to SEQ ID NO: 32
  Description of Artificial Sequence Synthetic oligonucleotide

SEQ ID NO: 41 - DNA, 58 bp, Unknown; identical to SEQ ID NO: 32
  Description of Unknown T4 phage sequence

SEQ ID NO: 42 - DNA, 58 bp, Unknown
  Description of Unknown T4 phage sequence
cgctgcgtggttccgatccgaacaacttccaaccggtaatgggattcaaaactcgtta

SEQ ID NO: 43 - DNA, 58 bp, Unknown; identical to SEQ ID NO: 32
  Description of Unknown T4 phage sequence

SEQ ID NO: 44 - DNA, 58 bp, Unknown; identical to SEQ ID NO: 32
  Description of Unknown T4 phage sequence

SEQ ID NO: 45 - DNA, 58 bp, Unknown; identical to SEQ ID NO: 42
  Description of Unknown T4 phage sequence

SEQ ID NO: 46 - DNA, 58 bp, Unknown; identical to SEQ ID NO: 42
  Description of Unknown T4 phage sequence

SEQ ID NO: 47 - DNA, 58 bp, Unknown; identical to SEQ ID NO: 32
  Description of Unknown T4 phage sequence

SEQ ID NO: 48 - DNA, 58 bp, Unknown; identical to SEQ ID NO: 32
  Description of Unknown T4 phage sequence

SEQ ID NO: 49 - DNA, 58 bp, Unknown; identical to SEQ ID NO: 32
  Description of Unknown T4 phage sequence

SEQ ID NO: 50 - DNA, 58 bp, Unknown; identical to SEQ ID NO: 32
  Description of Unknown T4 phage sequence

SEQ ID NO: 51 - DNA, 58 bp, Unknown
  Description of Unknown T4 phage sequence
ccatcacggcgctgcaaccaatagtcttcggtcatagacatattatgctgttgatttt

SEQ ID NO: 52 - DNA, 58 bp, Artificial Sequence
  Description of Artificial Sequence Synthetic oligonucleotide
ccatcacggcgctgcaaccaatagtcttcagtcatagacatattatgctgttgatttt

SEQ ID NO: 53 - DNA, 58 bp, Artificial Sequence
  Description of Artificial Sequence Synthetic oligonucleotide
ccatcacggcgctgcaaccaatagtcttctgtcatagacatattatgctgttgatttt

SEQ ID NO: 54 - DNA, 58 bp, Artificial Sequence
  Description of Artificial Sequence Synthetic oligonucleotide
ccatcacggcgctgcaaccaatagtcttcgatcatagacatattatgctgttgatttt

SEQ ID NO: 55 - DNA, 58 bp, Artificial Sequence; identical to SEQ ID NO: 53
  Description of Artificial Sequence Synthetic oligonucleotide

SEQ ID NO: 56 - DNA, 58 bp, Artificial Sequence; identical to SEQ ID NO: 53
  Description of Artificial Sequence Synthetic oligonucleotide

SEQ ID NO: 57 - DNA, 58 bp, Artificial Sequence; identical to SEQ ID NO: 53
  Description of Artificial Sequence Synthetic oligonucleotide

SEQ ID NO: 58 - DNA, 58 bp, Artificial Sequence; identical to SEQ ID NO: 53
  Description of Artificial Sequence Synthetic oligonucleotide

SEQ ID NO: 59 - DNA, 58 bp, Artificial Sequence; identical to SEQ ID NO: 54
  Description of Artificial Sequence Synthetic oligonucleotide

SEQ ID NO: 60 - DNA, 58 bp, Artificial Sequence; identical to SEQ ID NO: 53
  Description of Artificial Sequence Synthetic oligonucleotide

SEQ ID NO: 61 - DNA, 58 bp, Artificial Sequence; identical to SEQ ID NO: 52
  Description of Artificial Sequence Synthetic oligonucleotide

SEQ ID NO: 62 - DNA, 58 bp, Unknown; identical to SEQ ID NO: 51
  Description of Unknown T4 phage sequence

SEQ ID NO: 63 - DNA, 58 bp, Unknown; identical to SEQ ID NO: 51
  Description of Unknown T4 phage sequence

SEQ ID NO: 64 - DNA, 58 bp, Unknown; identical to SEQ ID NO: 51
  Description of Unknown T4 phage sequence

SEQ ID NO: 65 - DNA, 58 bp, Unknown; identical to SEQ ID NO: 52
  Description of Unknown T4 phage sequence

SEQ ID NO: 66 - DNA, 58 bp, Unknown; identical to SEQ ID NO: 51
  Description of Unknown T4 phage sequence

SEQ ID NO: 67 - DNA, 58 bp, Unknown; identical to SEQ ID NO: 51
  Description of Unknown T4 phage sequence

SEQ ID NO: 68 - DNA, 58 bp, Unknown; identical to SEQ ID NO: 51
  Description of Unknown T4 phage sequence

SEQ ID NO: 69 - DNA, 58 bp, Unknown; identical to SEQ ID NO: 53
  Description of Unknown T4 phage sequence

SEQ ID NO: 70 - DNA, 58 bp, Unknown; identical to SEQ ID NO: 51
  Description of Unknown T4 phage sequence

SEQ ID NO: 71 - DNA, 58 bp, Unknown; identical to SEQ ID NO: 51
  Description of Unknown T4 phage sequence

SEQ ID NO: 72 - DNA, 41 bp, Unknown
  Description of Unknown T4 phage sequence
atccgaagaacttccaaccggtaatgggattcaaaactcgt

SEQ ID NO: 73 - DNA, 41 bp, Artificial Sequence
  Description of Artificial Sequence Synthetic oligonucleotide
atccgaagaacttctagccagtgatgggattcaaaactcgt

SEQ ID NO: 74 - DNA, 41 bp, Artificial Sequence; identical to SEQ ID NO: 73
  Description of Artificial Sequence Synthetic oligonucleotide

SEQ ID NO: 75 - DNA, 42 bp, Artificial Sequence
  Description of Artificial Sequence Synthetic oligonucleotide
gatccgaagaacttctagccagtgatgggattcaaaactcgt

SEQ ID NO: 76 - DNA, 123 bp, Unknown
  Description of Unknown T4 phage sequence
aaatgtcccccgaagaccagctcgaaattaaatttgagcttgtggatatatttcatttcgtattaaatatgtttgttggccttggaatgaatgcggaagaaatctttaaactttattatctga

SEQ ID NO: 77 - DNA, 123 bp, Artificial Sequence
  Description of Artificial Sequence Synthetic polynucleotide
aaatgtctccggaggatcagctggaaatcaaattcgaactggttgacatcttccacttcgttctgaacatgttcgttggtctgggtatgaacgctgaagaaatcttcaaactgtactacctga

SEQ ID NO: 78 - DNA, 123 bp, Artificial Sequence; identical to SEQ ID NO: 77
  Description of Artificial Sequence Synthetic polynucleotide

SEQ ID NO: 79 - DNA, 123 bp, Artificial Sequence; identical to SEQ ID NO: 77
  Description of Artificial Sequence Synthetic polynucleotide

SEQ ID NO: 80 - DNA, 80 bp, Unknown
  Description of Unknown T4 phage sequence
aatgcgtgaaagaggaacacgtaatgccatataaagagcttgtctaaaccaacgaatatcttccatattgccagtattat

SEQ ID NO: 81 - DNA, 80 bp, Artificial Sequence
  Description of Artificial Sequence Synthetic oligonucleotide
aatgcgtgaaagaggaacctataatgccatataaagagcttgtctaaaccatcttatgtcttccatattgccagtattat

SEQ ID NO: 82 - DNA, 80 bp, Artificial Sequence; identical to SEQ ID NO: 81
  Description of Artificial Sequence Synthetic oligonucleotide

SEQ ID NO: 83 - DNA, 35 bp, Artificial Sequence
  Description of Artificial Sequence Synthetic primer
tcatagatctcacaaatgactatcaaaactaaagc

SEQ ID NO: 84 - DNA, 33 bp, Artificial Sequence
  Description of Artificial Sequence Synthetic primer
ctaggatcccgttagagattagatacctttaac

SEQ ID NO: 85 - DNA, 69 bp, Artificial Sequence
  Description of Artificial Sequence Synthetic primer
gctccatatgtagctctgactccgctgcgtggttccgatccgaagaacttctagccagtgatgggattc

SEQ ID NO: 86 - DNA, 32 bp, Artificial Sequence
  Description of Artificial Sequence Synthetic primer
actctcgagaatcatatacaaacggagtgatg

SEQ ID NO: 87 - DNA, 32 bp, Artificial Sequence
  Description of Artificial Sequence Synthetic primer
atcgagatctctccatatgtagctctgactcc

SEQ ID NO: 88 - DNA, 32 bp, Artificial Sequence
  Description of Artificial Sequence Synthetic primer
atcgagatctcactgtaggttataaaggtccg

SEQ ID NO: 89 - DNA, 32 bp, Artificial Sequence
  Description of Artificial Sequence Synthetic primer
atcgagatctctaccggctttagcactgatac

SEQ ID NO: 90 - DNA, 31 bp, Artificial Sequence
  Description of Artificial Sequence Synthetic primer
atcgagatctaaatcaaccgtgaagttgttg

SEQ ID NO: 91 - DNA, 29 bp, Artificial Sequence
  Description of Artificial Sequence Synthetic primer
gtgctcgagcggattctgcaaatgggttg

SEQ ID NO: 92 - DNA, 29 bp, Artificial Sequence
  Description of Artificial Sequence Synthetic primer
gtgctcgagcagaatagaaggcataccgc

SEQ ID NO: 93 - DNA, 29 bp, Artificial Sequence
  Description of Artificial Sequence Synthetic primer
gtgctcgagaagggaacccgaaggttccc

SEQ ID NO: 94 - DNA, 28 bp, Artificial Sequence
  Description of Artificial Sequence Synthetic primer
gtgctcgagcaaaggcaaaaacaggcgt

SEQ ID NO: 95 - DNA, 33 bp, Artificial Sequence
  Description of Artificial Sequence Synthetic primer
gtgctcgagcctttcggtccctttttatttaac

SEQ ID NO: 96 - DNA, 23 bp, Artificial Sequence
  Description of Artificial Sequence Synthetic primer
gtttaaaaagtatagcagtcttg

SEQ ID NO: 97 - DNA, 40 bp, Artificial Sequence
  Description of Artificial Sequence Synthetic primer
agcgaattcggaaaatacctaataagatacaaccgctgag

SEQ ID NO: 98 - DNA, 30 bp, Artificial Sequence
  Description of Artificial Sequence Synthetic primer
gtttctagagtatataatggcaccaactcg

SEQ ID NO: 99 - DNA, 37 bp, Artificial Sequence
  Description of Artificial Sequence Synthetic primer
tccgaattcttattagtgttttaccttgttcagctgc

SEQ ID NO: 100 - DNA, 29 bp, Artificial Sequence
  Description of Artificial Sequence Synthetic primer
gtttctagaggctaatgttacgggtgctc

SEQ ID NO: 101 - DNA, 23 bp, Unknown
  Description of Unknown T4 phage sequence
ccgaagactattggttgcagcgc

SEQ ID NO: 102 - protein, 7 aa, Unknown
  Description of Unknown T4 phage sequence
EDYWLQR

SEQ ID NO: 103 - DNA, 23 bp, Unknown
  Description of Unknown T4 phage sequence
gcaatatggaagatattcgttgg

SEQ ID NO: 104 - protein, 7 aa, Unknown
  Description of Unknown T4 phage sequence
NMEDIRW

SEQ ID NO: 105 - DNA, 23 bp, Unknown
  Description of Unknown T4 phage sequence
ccagagcggtatgccttctattc

SEQ ID NO: 106 - protein, 7 aa, Unknown
  Description of Unknown T4 phage sequence
QSGMPSI

SEQ ID NO: 107 - DNA, 23 bp, Unknown
  Description of Unknown T4 phage sequence
aagaacttccaaccggtaatggg

SEQ ID NO: 108 - protein, 7 aa, Unknown
  Description of Unknown T4 phage sequence
KNFQPVM

SEQ ID NO: 109 - DNA, 23 bp, Unknown
  Description of Unknown T4 phage sequence
gcgctgcaaccaatagtcttcgg

SEQ ID NO: 110 - DNA, 23 bp, Artificial Sequence
  Description of Artificial Sequence Synthetic oligonucleotide
gcgctgcaaccaatagtcttcgg

SEQ ID NO: 111 - DNA, 23 bp, Artificial Sequence
  Description of Artificial Sequence Synthetic oligonucleotide
gcgctgcaaccaatagtcttcgg

SEQ ID NO: 112 - DNA, 23 bp, Artificial Sequence
  Description of Artificial Sequence Synthetic oligonucleotide
gcgctgcaaccaatagtcttcgg

SEQ ID NO: 113 - DNA, 23 bp, Artificial Sequence
  Description of Artificial Sequence Synthetic oligonucleotide
gcgctgcatccaatagtcttcgg

SEQ ID NO: 114 - DNA, 23 bp, Artificial Sequence
  Description of Artificial Sequence Synthetic oligonucleotide
gcgctgcaaccaatagtcttcgg

SEQ ID NO: 115 - DNA, 23 bp, Artificial Sequence
  Description of Artificial Sequence Synthetic oligonucleotide
gcgctgcaaccaatagtcttcgg

SEQ ID NO: 116 - DNA, 23 bp, Artificial Sequence
  Description of Artificial Sequence Synthetic oligonucleotide
gcgctgcaaccaatagtcttcgg

SEQ ID NO: 117 - DNA, 23 bp, Artificial Sequence
  Description of Artificial Sequence Synthetic oligonucleotide
gcgctgcaaccaatagtcttcgg

SEQ ID NO: 118 - DNA, 23 bp, Artificial Sequence
  Description of Artificial Sequence Synthetic oligonucleotide
gcgctgcaaccaatagtcttcgg

SEQ ID NO: 119 - DNA, 23 bp, Artificial Sequence
  Description of Artificial Sequence Synthetic oligonucleotide
gcgctgcaaccaatagtcttcgg

SEQ ID NO: 120 - DNA, 23 bp, Unknown
  Description of Unknown T4 phage sequence
gaatagaaggcataccgctctgg

SEQ ID NO: 121 - DNA, 23 bp, Artificial Sequence
  Description of Artificial Sequence Synthetic oligonucleotide
gaatagaaggcataccgctctgg

SEQ ID NO: 122 - DNA, 23 bp, Artificial Sequence
  Description of Artificial Sequence Synthetic oligonucleotide
gaatagaaggcataccgctctgg

SEQ ID NO: 123 - DNA, 23 bp, Artificial Sequence
  Description of Artificial Sequence Synthetic oligonucleotide
gaatagaaggcataccgctctgg

SEQ ID NO: 124 - DNA, 23 bp, Artificial Sequence
  Description of Artificial Sequence Synthetic oligonucleotide
gaatagaaggcatacctctctgg

SEQ ID NO: 125 - DNA, 23 bp, Artificial Sequence
  Description of Artificial Sequence Synthetic oligonucleotide
gaatagaaggcataccgctctgg

SEQ ID NO: 126 - DNA, 23 bp, Artificial Sequence
  Description of Artificial Sequence Synthetic oligonucleotide
gaatagaaggcataccgctctgg

SEQ ID NO: 127 - DNA, 23 bp, Artificial Sequence
  Description of Artificial Sequence Synthetic oligonucleotide
gaatagaaggcataccgctctgg

SEQ ID NO: 128 - DNA, 23 bp, Artificial Sequence
  Description of Artificial Sequence Synthetic oligonucleotide
gaatagaaggcataccgctctgg

SEQ ID NO: 129 - DNA, 23 bp, Artificial Sequence
  Description of Artificial Sequence Synthetic oligonucleotide
gaatagaaggcataccgctctgg

SEQ ID NO: 130 - DNA, 23 bp, Artificial Sequence
  Description of Artificial Sequence Synthetic oligonucleotide
gaatagaaggcataccgctctgg

SEQ ID NO: 131 - DNA, 23 bp, Unknown
  Description of Unknown T4 phage sequence
gcaatatggaagatattcgttgg

SEQ ID NO: 132 - DNA, 23 bp, Artificial Sequence
  Description of Artificial Sequence Synthetic oligonucleotide
gcaatatggaagatattcgttgt

SEQ ID NO: 133 - DNA, 23 bp, Artificial Sequence
  Description of Artificial Sequence Synthetic oligonucleotide
gcaatatggaagatattcgtttg

SEQ ID NO: 134 - DNA, 23 bp, Artificial Sequence
  Description of Artificial Sequence Synthetic oligonucleotide
gcaatatggaagatattcgtttg

SEQ ID NO: 135 - DNA, 23 bp, Artificial Sequence
  Description of Artificial Sequence Synthetic oligonucleotide
gcaatatggaagatattcgtttg

SEQ ID NO: 136 - DNA, 23 bp, Artificial Sequence
  Description of Artificial Sequence Synthetic oligonucleotide
gcaatatggaagatattcgtttg

SEQ ID NO: 137 - DNA, 23 bp, Artificial Sequence
  Description of Artificial Sequence Synthetic oligonucleotide
gcaatatggaagatattcgtttg

SEQ ID NO: 138 - DNA, 23 bp, Artificial Sequence
  Description of Artificial Sequence Synthetic oligonucleotide
gcaatatggaagatattcgtttg

SEQ ID NO: 139 - DNA, 23 bp, Artificial Sequence
  Description of Artificial Sequence Synthetic oligonucleotide
gcaatatggaagatattcgttgt

SEQ ID NO: 140 - DNA, 23 bp, Artificial Sequence
  Description of Artificial Sequence Synthetic oligonucleotide
gcaatatggaagatattcgtttg

SEQ ID NO: 141 - DNA, 23 bp, Artificial Sequence
  Description of Artificial Sequence Synthetic oligonucleotide
gcaatatggaagatattcgtttg

SEQ ID NO: 142 - DNA, 23 bp, Unknown
  Description of Unknown T4 phage sequence
aagaacttccaaccggtaatggg

SEQ ID NO: 143 - DNA, 23 bp, Artificial Sequence
  Description of Artificial Sequence Synthetic oligonucleotide
aagaacttccaaccgttaatggg

SEQ ID NO: 144 - DNA, 23 bp, Artificial Sequence
  Description of Artificial Sequence Synthetic oligonucleotide
aagaacttccaaccggtaatggt

SEQ ID NO: 145 - DNA, 23 bp, Artificial Sequence
  Description of Artificial Sequence Synthetic oligonucleotide
aagaacttccaaccggtaatggc

SEQ ID NO: 146 - DNA, 23 bp, Artificial Sequence
  Description of Artificial Sequence Synthetic oligonucleotide
aaaaacttccaaccggtaatggg

SEQ ID NO: 147 - DNA, 23 bp, Artificial Sequence
  Description of Artificial Sequence Synthetic oligonucleotide
aagaacttccaaccggtaatggt

SEQ ID NO: 148 - DNA, 23 bp, Artificial Sequence
  Description of Artificial Sequence Synthetic oligonucleotide
aagaacttccaacctgtaatggg

SEQ ID NO: 149 - DNA, 23 bp, Artificial Sequence
  Description of Artificial Sequence Synthetic oligonucleotide
aagaacttccaaccggtgatggg

SEQ ID NO: 150 - DNA, 23 bp, Artificial Sequence
  Description of Artificial Sequence Synthetic oligonucleotide
aggaacttccaaccggtaatggg

SEQ ID NO: 151 - DNA, 23 bp, Artificial Sequence
  Description of Artificial Sequence Synthetic oligonucleotide
aagaacttccaaccggtgatggg

SEQ ID NO: 152 - DNA, 23 bp, Artificial Sequence
  Description of Artificial Sequence Synthetic oligonucleotide
aaaaacttccaaccggtaatggg

SEQ ID NO: 153 - DNA, 23 bp, Unknown
  Description of Unknown T4 phage sequence
gcgctgcaaccaatagtcttcgg

SEQ ID NO: 154 - DNA, 23 bp, Artificial Sequence
  Description of Artificial Sequence Synthetic oligonucleotide
gcgctgcaaccaatagtcttcgg

SEQ ID NO: 155 - DNA, 23 bp, Artificial Sequence
  Description of Artificial Sequence Synthetic oligonucleotide
gcgctgcaaccaatagtcttcgg

SEQ ID NO: 156 - DNA, 23 bp, Artificial Sequence
  Description of Artificial Sequence Synthetic oligonucleotide
gcgctgcaaccaatagtcttcgg

SEQ ID NO: 157 - DNA, 23 bp, Artificial Sequence
  Description of Artificial Sequence Synthetic oligonucleotide
gcgctgcaaccaatagtcttcgg

SEQ ID NO: 158 - DNA, 23 bp, Artificial Sequence
  Description of Artificial Sequence Synthetic oligonucleotide
gcgctgcaaccaatagtcttcgg

SEQ ID NO: 159 - DNA, 23 bp, Artificial Sequence
  Description of Artificial Sequence Synthetic oligonucleotide
gcgctgcaaccaatagtcttcgg

SEQ ID NO: 160 - DNA, 23 bp, Artificial Sequence
  Description of Artificial Sequence Synthetic oligonucleotide
gcgctgcaaccaatagtcttcgg

SEQ ID NO: 161 - DNA, 23 bp, Artificial Sequence
  Description of Artificial Sequence Synthetic oligonucleotide
gcgctgcatccaatagtcttcgg

SEQ ID NO: 162 - DNA, 23 bp, Artificial Sequence
  Description of Artificial Sequence Synthetic oligonucleotide
gcgctgcaaccaatagtcttcgg

SEQ ID NO: 163 - DNA, 23 bp, Artificial Sequence
  Description of Artificial Sequence Synthetic oligonucleotide
gcgctgcaaccaatagtcttcgg

SEQ ID NO: 164 - DNA, 23 bp, Artificial Sequence
  Description of Artificial Sequence Synthetic oligonucleotide
gcgctgcaaccaatagtcttcgg

SEQ ID NO: 165 - DNA, 23 bp, Artificial Sequence
  Description of Artificial Sequence Synthetic oligonucleotide
gcgctgcaaccaatagtcttcag

SEQ ID NO: 166 - DNA, 23 bp, Artificial Sequence
  Description of Artificial Sequence Synthetic oligonucleotide
gcgctgcaaccaatagtcttcgc

SEQ ID NO: 167 - DNA, 23 bp, Artificial Sequence
  Description of Artificial Sequence Synthetic oligonucleotide
gcgctgcaaccaatagtcttcgg

SEQ ID NO: 168 - DNA, 23 bp, Artificial Sequence
  Description of Artificial Sequence Synthetic oligonucleotide
gcgctgcaaccaatagtcttcga

SEQ ID NO: 169 - DNA, 23 bp, Artificial Sequence
  Description of Artificial Sequence Synthetic oligonucleotide
gcgctgcaaccagtagtcttcgg

SEQ ID NO: 170 - DNA, 23 bp, Artificial Sequence
  Description of Artificial Sequence Synthetic oligonucleotide
gcgctgcaaccaatagtcttcgg

SEQ ID NO: 171 - DNA, 23 bp, Artificial Sequence
  Description of Artificial Sequence Synthetic oligonucleotide
gcgctgcatccaatagtcttcgg

SEQ ID NO: 172 - DNA, 23 bp, Artificial Sequence
  Description of Artificial Sequence Synthetic oligonucleotide
gcgctgcaaccaatagtcttcgg

SEQ ID NO: 173 - DNA, 23 bp, Artificial Sequence
  Description of Artificial Sequence Synthetic oligonucleotide
gcgctgcaaccaatagtcttcgg

SEQ ID NO: 174 - DNA, 23 bp, Artificial Sequence
  Description of Artificial Sequence Synthetic oligonucleotide
gcgctgcaaccaatagtcttcag

SEQ ID NO: 175 - DNA, 23 bp, Artificial Sequence
  Description of Artificial Sequence Synthetic oligonucleotide
gcgctgcaaccaatagtcttcag

SEQ ID NO: 176 - DNA, 23 bp, Artificial Sequence
  Description of Artificial Sequence Synthetic oligonucleotide
gcgctgcaaccaatagtcttcgc

SEQ ID NO: 177 - DNA, 23 bp, Artificial Sequence
  Description of Artificial Sequence Synthetic oligonucleotide
gcgctgtaaccaatagtcttcgg

SEQ ID NO: 178 - DNA, 23 bp, Artificial Sequence
  Description of Artificial Sequence Synthetic oligonucleotide
gcgctgcaaccaatagtcttcga

SEQ ID NO: 179 - DNA, 23 bp, Artificial Sequence
  Description of Artificial Sequence Synthetic oligonucleotide
gcgctgcaaccagtagtcttcgg

SEQ ID NO: 180 - DNA, 23 bp, Artificial Sequence
  Description of Artificial Sequence Synthetic oligonucleotide
gcgctgcagccaatagtcttcgg

SEQ ID NO: 181 - DNA, 23 bp, Artificial Sequence
  Description of Artificial Sequence Synthetic oligonucleotide
gcgctgcatccaatagtcttcgg

SEQ ID NO: 182 - DNA, 23 bp, Artificial Sequence
  Description of Artificial Sequence Synthetic oligonucleotide
gcgctgtaaccaatagtcttcgg

SEQ ID NO: 183 - DNA, 23 bp, Artificial Sequence
  Description of Artificial Sequence Synthetic oligonucleotide
gcgctgcaaccaatagtcttcgg

SEQ ID NO: 184 - DNA, 23 bp, Artificial Sequence
  Description of Artificial Sequence Synthetic oligonucleotide
gcgctgcaaccaatagtcttcag

SEQ ID NO: 185 - DNA, 23 bp, Artificial Sequence
  Description of Artificial Sequence Synthetic oligonucleotide
gcgctgcaaccaatagtcttcag

SEQ ID NO: 186 - DNA, 23 bp, Artificial Sequence
  Description of Artificial Sequence Synthetic oligonucleotide
gcgctgcaaccaatagtcttcgc

SEQ ID NO: 187 - DNA, 23 bp, Artificial Sequence
  Description of Artificial Sequence Synthetic oligonucleotide
gcgctgtaaccaatagtcttcgg

SEQ ID NO: 188 - DNA, 23 bp, Artificial Sequence
  Description of Artificial Sequence Synthetic oligonucleotide
gcgctgcaaccaatagtcttcga

SEQ ID NO: 189 - DNA, 23 bp, Artificial Sequence
  Description of Artificial Sequence Synthetic oligonucleotide
gcgctgcaaccagtagtcttcgg

SEQ ID NO: 190 - DNA, 23 bp, Artificial Sequence
  Description of Artificial Sequence Synthetic oligonucleotide
gcgctgcagccaatagtcttcgg

SEQ ID NO: 191 - DNA, 23 bp, Artificial Sequence
  Description of Artificial Sequence Synthetic oligonucleotide
gcgctgcatccaatagtcttcgg

SEQ ID NO: 192 - DNA, 23 bp, Artificial Sequence
  Description of Artificial Sequence Synthetic oligonucleotide
gcgctgtaaccaatagtcttcgg

SEQ ID NO: 193 - DNA, 23 bp, Artificial Sequence
  Description of Artificial Sequence Synthetic oligonucleotide
gcgctgtaaccaatagtcttcgg

SEQ ID NO: 194 - DNA, 23 bp, Unknown
  Description of Unknown T4 phage sequence
gcgctgcaaccaatagtcttcgg

SEQ ID NO: 195 - DNA, 23 bp, Artificial Sequence
  Description of Artificial Sequence Synthetic oligonucleotide
gcgctgcaaccaatagtcttcga

SEQ ID NO: 196 - DNA, 23 bp, Artificial Sequence
  Description of Artificial Sequence Synthetic oligonucleotide
gcgctgcaaccaatagtcttcgg

SEQ ID NO: 197 - DNA, 23 bp, Artificial Sequence
  Description of Artificial Sequence Synthetic oligonucleotide
gcgctgcaaccaatagtcttcga

SEQ ID NO: 198 - DNA, 23 bp, Artificial Sequence
  Description of Artificial Sequence Synthetic oligonucleotide
gcgctgcaaccaatagtcttcga

SEQ ID NO: 199 - DNA, 23 bp, Artificial Sequence
  Description of Artificial Sequence Synthetic oligonucleotide
gcgctgcagccaatagtcttcgg

SEQ ID NO: 200 - DNA, 23 bp, Artificial Sequence
  Description of Artificial Sequence Synthetic oligonucleotide
gcgctgtaaccaatagtcttcgg

SEQ ID NO: 201 - DNA, 23 bp, Artificial Sequence
  Description of Artificial Sequence Synthetic oligonucleotide
gcgctgcaaccaatagtcttcga

SEQ ID NO: 202 - DNA, 23 bp, Artificial Sequence
  Description of Artificial Sequence Synthetic oligonucleotide
gcgctgtaaccaatagtcttcgg

SEQ ID NO: 203 - DNA, 23 bp, Artificial Sequence
  Description of Artificial Sequence Synthetic oligonucleotide
gcgctgcaaccaatagtcttcga

SEQ ID NO: 204 - DNA, 23 bp, Artificial Sequence
  Description of Artificial Sequence Synthetic oligonucleotide
gcgctgcaaccaatagtcttcag

SEQ ID NO: 205 - DNA, 23 bp, Unknown
  Description of Unknown T4 phage sequence
gcgctgcaaccaatagtcttcgg

SEQ ID NO: 206 - DNA, 23 bp, Artificial Sequence
  Description of Artificial Sequence Synthetic oligonucleotide
gcgctgcaaccaatagtcttcga

SEQ ID NO: 207 - DNA, 23 bp, Artificial Sequence
  Description of Artificial Sequence Synthetic oligonucleotide
gcgctgcaaccaatagtcttcgg

SEQ ID NO: 208 - DNA, 23 bp, Artificial Sequence
  Description of Artificial Sequence Synthetic oligonucleotide
gcgctgcaaccaatagtcttcga

SEQ ID NO: 209 - DNA, 23 bp, Artificial Sequence
  Description of Artificial Sequence Synthetic oligonucleotide
gcgctgcaaccaatagtcttcga

SEQ ID NO: 210 - DNA, 23 bp, Artificial Sequence
  Description of Artificial Sequence Synthetic oligonucleotide
gcgctgcagccaatagtcttcgg

SEQ ID NO: 211 - DNA, 23 bp, Artificial Sequence
  Description of Artificial Sequence Synthetic oligonucleotide
gcgctgtaaccaatagtcttcgg

SEQ ID NO: 212 - DNA, 23 bp, Artificial Sequence
  Description of Artificial Sequence Synthetic oligonucleotide
gcgctgcaaccaatagtcttcga

SEQ ID NO: 213 - DNA, 23 bp, Artificial Sequence
  Description of Artificial Sequence Synthetic oligonucleotide
gcgctgtaaccaatagtcttcgg

SEQ ID NO: 214 - DNA, 23 bp, Artificial Sequence
  Description of Artificial Sequence Synthetic oligonucleotide
gcgctgcaaccaatagtcttcga

SEQ ID NO: 215 - DNA, 23 bp, Artificial Sequence
  Description of Artificial Sequence Synthetic oligonucleotide
gcgctgcaaccaatagtcttcag

SEQ ID NO: 216 - DNA, 23 bp, Unknown
  Description of Unknown T4 phage sequence
gcgctgcaaccaatagtcttcgg

SEQ ID NO: 217 - DNA, 23 bp, Artificial Sequence
  Description of Artificial Sequence Synthetic oligonucleotide
gcgctgcaaccaatagtcttcga

SEQ ID NO: 218 - DNA, 23 bp, Artificial Sequence
  Description of Artificial Sequence Synthetic oligonucleotide
gcgctgtaaccaatagtcttcgg

SEQ ID NO: 219 - DNA, 23 bp, Unknown
  Description of Unknown T4 phage sequence
gaatagaaggcataccgctctgg

SEQ ID NO: 220 - DNA, 23 bp, Artificial Sequence
  Description of Artificial Sequence Synthetic oligonucleotide
gaatagaaggcataccgctctgg

SEQ ID NO: 221 - DNA, 23 bp, Artificial Sequence
  Description of Artificial Sequence Synthetic oligonucleotide
gaatagaaggcataccgctctgg

SEQ ID NO: 222 - DNA, 23 bp, Artificial Sequence
  Description of Artificial Sequence Synthetic oligonucleotide
gaatagaaggcataccgctctgg

SEQ ID NO: 223 - DNA, 23 bp, Artificial Sequence
  Description of Artificial Sequence Synthetic oligonucleotide
gaatagaaggcatacctctctgg

SEQ ID NO: 224 - DNA, 23 bp, Artificial Sequence
  Description of Artificial Sequence Synthetic oligonucleotide
gaatagaaggcataccgctctgg

SEQ ID NO: 225 - DNA, 23 bp, Artificial Sequence
  Description of Artificial Sequence Synthetic oligonucleotide
gaatagaaggcataccgctctgg

SEQ ID NO: 226 - DNA, 23 bp, Artificial Sequence
  Description of Artificial Sequence Synthetic oligonucleotide
gaatagaaggcataccgctctgg

SEQ ID NO: 227 - DNA, 23 bp, Artificial Sequence
  Description of Artificial Sequence Synthetic oligonucleotide
gaatagaaggcataccgctctgg

SEQ ID NO: 228 - DNA, 23 bp, Artificial Sequence
  Description of Artificial Sequence Synthetic oligonucleotide
gaatagaaggcataccgctctgg

SEQ ID NO: 229 - DNA, 23 bp, Artificial Sequence
  Description of Artificial Sequence Synthetic oligonucleotide
gaatagaaggcataccgctctgg

SEQ ID NO: 230 - DNA, 23 bp, Artificial Sequence
  Description of Artificial Sequence Synthetic oligonucleotide
gaatagaaggcataccgctctgg

SEQ ID NO: 231 - DNA, 23 bp, Artificial Sequence
  Description of Artificial Sequence Synthetic oligonucleotide
gaatagaaggcataccgctctgg

SEQ ID NO: 232 - DNA, 23 bp, Artificial Sequence
  Description of Artificial Sequence Synthetic oligonucleotide
gaatagaaggcataccgctctgg

SEQ ID NO: 233 - DNA, 23 bp, Artificial Sequence
  Description of Artificial Sequence Synthetic oligonucleotide
gaatagaaggcatacctctctgg

SEQ ID NO: 234 - DNA, 23 bp, Artificial Sequence
  Description of Artificial Sequence Synthetic oligonucleotide
gaatagaaggcatacctctctgg

SEQ ID NO: 235 - DNA, 23 bp, Artificial Sequence
  Description of Artificial Sequence Synthetic oligonucleotide
gaatagaaggcataccgctctgg

SEQ ID NO: 236 - DNA, 23 bp, Artificial Sequence
  Description of Artificial Sequence Synthetic oligonucleotide
gaatagaaggcataccgctctgg

SEQ ID NO: 237 - DNA, 23 bp, Artificial Sequence
  Description of Artificial Sequence Synthetic oligonucleotide
gaatagaaggcatacctctctgg

SEQ ID NO: 238 - DNA, 23 bp, Artificial Sequence
  Description of Artificial Sequence Synthetic oligonucleotide
gaatagaaggcataccgctctgg

SEQ ID NO: 239 - DNA, 23 bp, Artificial Sequence
  Description of Artificial Sequence Synthetic oligonucleotide
gaatagaaggcatacctctctgg

SEQ ID NO: 240 - DNA, 23 bp, Artificial Sequence
  Description of Artificial Sequence Synthetic oligonucleotide
gaatagaaggcatacctctctgg

SEQ ID NO: 241 - DNA, 23 bp, Artificial Sequence
  Description of Artificial Sequence Synthetic oligonucleotide
gaatagaaggcatacctctctgg

SEQ ID NO: 242 - DNA, 23 bp, Artificial Sequence
  Description of Artificial Sequence Synthetic oligonucleotide
gaatagaaggcatacctctctgg

SEQ ID NO: 243 - DNA, 23 bp, Artificial Sequence
  Description of Artificial Sequence Synthetic oligonucleotide
gaatagaaggcatacctctctgg

SEQ ID NO: 244 - DNA, 23 bp, Artificial Sequence
  Description of Artificial Sequence Synthetic oligonucleotide
gaatagaaggcatacctctctgg

SEQ ID NO: 245 - DNA, 23 bp, Artificial Sequence
  Description of Artificial Sequence Synthetic oligonucleotide
gaatagaaggcatactgctctgg

SEQ ID NO: 246 - DNA, 23 bp, Artificial Sequence
  Description of Artificial Sequence Synthetic oligonucleotide
gaatagaaggcatacctctctgg

SEQ ID NO: 247 - DNA, 23 bp, Artificial Sequence
  Description of Artificial Sequence Synthetic oligonucleotide
gaatagaaggcatacctctctgg

SEQ ID NO: 248 - DNA, 23 bp, Artificial Sequence
  Description of Artificial Sequence Synthetic oligonucleotide
gaatagaaggcatacctctctgg

SEQ ID NO: 249 - DNA, 23 bp, Artificial Sequence
  Description of Artificial Sequence Synthetic oligonucleotide
gaatagaaggcatacctctctgg

SEQ ID NO: 250 - DNA, 24 bp, Unknown
  Description of Unknown T4 phage sequence
accgaagactattggttgcagcgc

SEQ ID NO: 251 - DNA, 24 bp, Unknown
  Description of Unknown T4 phage sequence
tggcttctgataaccaacgtcgcg

SEQ ID NO: 252 - DNA, 24 bp, Artificial Sequence
  Description of Artificial Sequence Synthetic oligonucleotide
tgacttctgataaccaacgtcgcg

SEQ ID NO: 253 - DNA, 24 bp, Artificial Sequence
  Description of Artificial Sequence Synthetic oligonucleotide
tcgcttctgataaccaacgtcgcg

SEQ ID NO: 254 - DNA, 24 bp, Artificial Sequence
  Description of Artificial Sequence Synthetic oligonucleotide
tggcttctgataaccaatgtcgcg

SEQ ID NO: 255 - DNA, 24 bp, Artificial Sequence
  Description of Artificial Sequence Synthetic oligonucleotide
tagcttctgataaccaacgtcgcg

SEQ ID NO: 256 - DNA, 24 bp, Artificial Sequence
  Description of Artificial Sequence Synthetic oligonucleotide
tggcttctgatgaccaacgtcgcg

SEQ ID NO: 257 - DNA, 24 bp, Artificial Sequence
  Description of Artificial Sequence Synthetic oligonucleotide
tggcttctgataaccgacgtcgcg

SEQ ID NO: 258 - DNA, 24 bp, Artificial Sequence
  Description of Artificial Sequence Synthetic oligonucleotide
tggcttctgataacctacgtcgcg

SEQ ID NO: 259 - DNA, 24 bp, Unknown
  Description of Unknown T4 phage sequence
ggcaatatggaagatattcgttgg

SEQ ID NO: 260 - DNA, 24 bp, Artificial Sequence
  Description of Artificial Sequence Synthetic oligonucleotide
ggcaatatggaagatattcgttgt

SEQ ID NO: 261 - DNA, 24 bp, Artificial Sequence
  Description of Artificial Sequence Synthetic oligonucleotide
ggcaatatggaagatattcgtttg

SEQ ID NO: 262 - DNA, 24 bp, Unknown
  Description of Unknown T4 phage sequence
aagaacttccaaccggtaatggga

SEQ ID NO: 263 - DNA, 24 bp, Artificial Sequence
  Description of Artificial Sequence Synthetic oligonucleotide
aagaacttccaaccgttaatggga

SEQ ID NO: 264 - DNA, 24 bp, Artificial Sequence
  Description of Artificial Sequence Synthetic oligonucleotide
aagaacttccaaccggtaatggta

SEQ ID NO: 265 - DNA, 24 bp, Artificial Sequence
  Description of Artificial Sequence Synthetic oligonucleotide
aagaacttccaaccggtaatggca

SEQ ID NO: 266 - DNA, 24 bp, Artificial Sequence
  Description of Artificial Sequence Synthetic oligonucleotide
aaaaacttccaaccggtaatggga

SEQ ID NO: 267 - DNA, 24 bp, Artificial Sequence
  Description of Artificial Sequence Synthetic oligonucleotide
aagaacttccaacctgtaatggga

SEQ ID NO: 268 - DNA, 24 bp, Artificial Sequence
  Description of Artificial Sequence Synthetic oligonucleotide
aagaacttccaaccggtgatggga

SEQ ID NO: 269 - DNA, 24 bp, Artificial Sequence
  Description of Artificial Sequence Synthetic oligonucleotide
aggaacttccaaccggtaatggga

SEQ ID NO: 270 - DNA, 24 bp, Unknown
  Description of Unknown T4 phage sequence
tccagagcggtatgccttctattc

SEQ ID NO: 271 - DNA, 24 bp, Unknown
  Description of Unknown T4 phage sequence
aggtctcgccatacggaagataag

SEQ ID NO: 272 - DNA, 24 bp, Artificial Sequence
  Description of Artificial Sequence Synthetic oligonucleotide
aggtctctccatacggaagataag

SEQ ID NO: 273 - DNA, 24 bp, Artificial Sequence
  Description of Artificial Sequence Synthetic oligonucleotide
aggtcttgccatacggaagataag

SEQ ID NO: 274 - DNA, 24 bp, Unknown
  Description of Unknown T4 phage sequence
accgaagactattggttgcagcgc

SEQ ID NO: 275 - protein, 8 aa, Unknown
  Description of Unknown T4 phage sequence
TEDYWLQR

SEQ ID NO: 276 - DNA, 24 bp, Unknown
  Description of Unknown T4 phage sequence
accgaagactattggttgcagcgc

SEQ ID NO: 277 - DNA, 24 bp, Artificial Sequence
  Description of Artificial Sequence Synthetic oligonucleotide
agcaaacacgatggggtgaagagc

SEQ ID NO: 278 - DNA, 24 bp, Artificial Sequence
  Description of Artificial Sequence Synthetic oligonucleotide
aactaaaaccatcggctgtagtgc

SEQ ID NO: 279 - DNA, 24 bp, Artificial Sequence
  Description of Artificial Sequence Synthetic oligonucleotide
atccaatacaataggatggagggc

SEQ ID NO: 280 - DNA, 24 bp, Artificial Sequence
  Description of Artificial Sequence Synthetic oligonucleotide
acggtagtcttttcgtggctgccc

SEQ ID NO: 281 - DNA, 24 bp, Artificial Sequence
  Description of Artificial Sequence Synthetic oligonucleotide
acaggaggctgttagtcgcggcac

SEQ ID NO: 282 - DNA, 24 bp, Artificial Sequence
  Description of Artificial Sequence Synthetic oligonucleotide
actgcagcctctttgtagccgctc

SEQ ID NO: 283 - DNA, 21 bp, Artificial Sequence
  Description of Artificial Sequence Synthetic oligonucleotide
gatgaatagtgcttccaccga

SEQ ID NO: 284 - DNA, 21 bp, Artificial Sequence
  Description of Artificial Sequence Synthetic oligonucleotide
gaggattactgattacaacgt

SEQ ID NO: 285 - DNA, 21 bp, Artificial Sequence
  Description of Artificial Sequence Synthetic oligonucleotide
gacgagtaatgttttcatcgg

SEQ ID NO: 286 - DNA, 24 bp, Unknown
  Description of Unknown T4 phage sequence
ggcaatatggaagatattcgttgg

SEQ ID NO: 287 - protein, 8 aa, Unknown
  Description of Unknown T4 phage sequence
GNMEDIRW

SEQ ID NO: 288 - DNA, 24 bp, Unknown
  Description of Unknown T4 phage sequence
ggcaatatggaagatattcgttgg

SEQ ID NO: 289 - DNA, 24 bp, Artificial Sequence
  Description of Artificial Sequence Synthetic oligonucleotide
gcctatttgcaacattttagtggg

SEQ ID NO: 290 - DNA, 24 bp, Artificial Sequence
  Description of Artificial Sequence Synthetic oligonucleotide
gacgatgtgaaaaatgtttgtcgg

SEQ ID NO: 291 - DNA, 24 bp, Artificial Sequence
  Description of Artificial Sequence Synthetic oligonucleotide
gtccatctgtaatatcttggtagg

SEQ ID NO: 292 - DNA, 24 bp, Artificial Sequence
  Description of Artificial Sequence Synthetic oligonucleotide
ggaattagggtagttagtccttcg

SEQ ID NO: 293 - DNA, 24 bp, Artificial Sequence
  Description of Artificial Sequence Synthetic oligonucleotide
ggtagtacgggaggtactcattag

SEQ ID NO: 294 - DNA, 24 bp, Artificial Sequence
  Description of Artificial Sequence Synthetic oligonucleotide
gggactaaggcagctaatcttttg

SEQ ID NO: 295 - DNA, 21 bp, Artificial Sequence
  Description of Artificial Sequence Synthetic oligonucleotide
aagatcgatgagatgcggtgc

SEQ ID NO: 296 - DNA, 21 bp, Artificial Sequence
  Description of Artificial Sequence Synthetic oligonucleotide
aacatagaggacatccgctga

SEQ ID NO: 297 - DNA, 21 bp, Artificial Sequence
  Description of Artificial Sequence Synthetic oligonucleotide
aaaattgacgaaatacgatgt

SEQ ID NO: 298 - DNA, 27 bp, Unknown
  Description of Unknown T4 phage sequence
atccagagcggtatgccttctattctg

SEQ ID NO: 299 - protein, 9 aa, Unknown
  Description of Unknown T4 phage sequence
IQSGMPSIL

SEQ ID NO: 300 - DNA, 27 bp, Unknown
  Description of Unknown T4 phage sequence
atccagagcggtatgccttctattctg

SEQ ID NO: 301 - DNA, 27 bp, Artificial Sequence
  Description of Artificial Sequence Synthetic oligonucleotide
ataaagtgccgtttgactgcttttatg

SEQ ID NO: 302 - DNA, 27 bp, Artificial Sequence
  Description of Artificial Sequence Synthetic oligonucleotide
atttagggcagtgtgtctcctgttttg

SEQ ID NO: 303 - DNA, 27 bp, Artificial Sequence
  Description of Artificial Sequence Synthetic oligonucleotide
atggagcgctgtctggctactcttgtg

SEQ ID NO: 304 - DNA, 21 bp, Artificial Sequence
  Description of Artificial Sequence Synthetic oligonucleotide
ctgaccgctaggcattatagt

SEQ ID NO: 305 - DNA, 21 bp, Artificial Sequence
  Description of Artificial Sequence Synthetic oligonucleotide
cggaacgatacgctttttact

SEQ ID NO: 306 - DNA, 21 bp, Artificial Sequence
  Description of Artificial Sequence Synthetic oligonucleotide
ccgatcgttaagcgttgtaat

SEQ ID NO: 307 - DNA, 21 bp, Artificial Sequence
  Description of Artificial Sequence Synthetic oligonucleotide
cacagagggatcccgtcgatg

SEQ ID NO: 308 - DNA, 21 bp, Artificial Sequence
  Description of Artificial Sequence Synthetic oligonucleotide
caaagtggcataccctccatc

SEQ ID NO: 309 - DNA, 21 bp, Artificial Sequence
  Description of Artificial Sequence Synthetic oligonucleotide
cataggggaattccatcaata

SEQ ID NO: 310 - DNA, 24 bp, Unknown
  Description of Unknown T4 phage sequence
aagaacttccaaccggtaatggga

SEQ ID NO: 311 - protein, 8 aa, Unknown
  Description of Unknown T4 phage sequence
KNFQPVMG

SEQ ID NO: 312 - DNA, 24 bp, Unknown
  Description of Unknown T4 phage sequence
aagaacttccaaccggtaatggga

SEQ ID NO: 313 - DNA, 24 bp, Artificial Sequence
  Description of Artificial Sequence Synthetic oligonucleotide
tagtacgtcaaaacgctattgcga

SEQ ID NO: 314 - DNA, 24 bp, Artificial Sequence
  Description of Artificial Sequence Synthetic oligonucleotide
gaggacctctaatcgatagtgaga

SEQ ID NO: 315 - DNA, 24 bp, Artificial Sequence
  Description of Artificial Sequence Synthetic oligonucleotide
cagcacatcgaagcgttactgtga

SEQ ID NO: 316 - DNA, 24 bp, Artificial Sequence
  Description of Artificial Sequence Synthetic oligonucleotide
atgatctgcctacagggaagggca

SEQ ID NO: 317 - DNA, 24 bp, Artificial Sequence
  Description of Artificial Sequence Synthetic oligonucleotide
aggagctcccgactggcaacggaa

SEQ ID NO: 318 - DNA, 24 bp, Artificial Sequence
  Description of Artificial Sequence Synthetic oligonucleotide
acgacctacccacgggaaaaggta

SEQ ID NO: 319 - DNA, 21 bp, Artificial Sequence
  Description of Artificial Sequence Synthetic oligonucleotide
aacaaattacatcccgttatc

SEQ ID NO: 320 - DNA, 21 bp, Artificial Sequence
  Description of Artificial Sequence Synthetic oligonucleotide
aaaaattttcagccagtgata

SEQ ID NO: 321 - DNA, 21 bp, Artificial Sequence
  Description of Artificial Sequence Synthetic oligonucleotide
aataagttgcaccctgtcatt

SEQ ID NO: 322 - DNA, 23 bp, Unknown
  Description of Unknown T4 phage sequence
gcgctgcaaccaatagtcttcgg

SEQ ID NO: 323 - DNA, 23 bp, Artificial Sequence
  Description of Artificial Sequence Synthetic oligonucleotide
gcgctgcaaccaatagtcttcga

SEQ ID NO: 324 - DNA, 23 bp, Artificial Sequence
  Description of Artificial Sequence Synthetic oligonucleotide
gcgctgcagccaatagtcttcgg

SEQ ID NO: 325 - DNA, 23 bp, Artificial Sequence
  Description of Artificial Sequence Synthetic oligonucleotide
gcgctgtaaccaatagtcttcgg

SEQ ID NO: 326 - DNA, 23 bp, Artificial Sequence
  Description of Artificial Sequence Synthetic oligonucleotide
gcgctgcaaccaatagtcttcag

SEQ ID NO: 327 - DNA, 23 bp, Unknown
  Description of Unknown T4 phage sequence
gcgctgcaaccaatagtcttcgg

SEQ ID NO: 328 - DNA, 23 bp, Artificial Sequence
  Description of Artificial Sequence Synthetic oligonucleotide
gcgctgtaaccaatagtcttcgg

SEQ ID NO: 329 - DNA, 23 bp, Artificial Sequence
  Description of Artificial Sequence Synthetic oligonucleotide
gcgctgcaaccaatagtcttcag

SEQ ID NO: 330 - DNA, 23 bp, Artificial Sequence
  Description of Artificial Sequence Synthetic oligonucleotide
gcgctgcaaccaatagtcttcag

SEQ ID NO: 331 - DNA, 23 bp, Artificial Sequence
  Description of Artificial Sequence Synthetic oligonucleotide
gcgctgcaaccaatagtcttcag

SEQ ID NO: 332 - DNA, 23 bp, Artificial Sequence
  Description of Artificial Sequence Synthetic oligonucleotide
gcgctgcagccaatagtcttcgg

SEQ ID NO: 333 - DNA, 23 bp, Artificial Sequence
  Description of Artificial Sequence Synthetic oligonucleotide
gcgctgcagccaatagtcttcgg

SEQ ID NO: 334 - DNA, 23 bp, Artificial Sequence
  Description of Artificial Sequence Synthetic oligonucleotide
gcgctgtaaccaatagtcttcgg

SEQ ID NO: 335 - DNA, 23 bp, Artificial Sequence
  Description of Artificial Sequence Synthetic oligonucleotide
gcgctgtaaccaatagtcttcgg

SEQ ID NO: 336 - DNA, 23 bp, Artificial Sequence
  Description of Artificial Sequence Synthetic oligonucleotide
gcgctgcagccaatagtcttcgg

SEQ ID NO: 337 - DNA, 23 bp, Artificial Sequence
  Description of Artificial Sequence Synthetic oligonucleotide
gcgctgcagccaatagtcttcgg

SEQ ID NO: 338 - DNA, 23 bp, Artificial Sequence
  Description of Artificial Sequence Synthetic oligonucleotide
gcgctgcagccaatagtcttcgg

SEQ ID NO: 339 - DNA, 23 bp, Artificial Sequence
  Description of Artificial Sequence Synthetic oligonucleotide
gcgctgcagccaatagtcttcgg

SEQ ID NO: 340 - DNA, 23 bp, Artificial Sequence
  Description of Artificial Sequence Synthetic oligonucleotide
gcgctgcagccaatagtcttcgg

SEQ ID NO: 341 - DNA, 23 bp, Artificial Sequence
  Description of Artificial Sequence Synthetic oligonucleotide
gcgctgcagccaatagtcttcgg

SEQ ID NO: 342 - DNA, 23 bp, Artificial Sequence
  Description of Artificial Sequence Synthetic oligonucleotide
gcgctgcaaccaatagtcttcag

SEQ ID NO: 343 - DNA, 23 bp, Artificial Sequence
  Description of Artificial Sequence Synthetic oligonucleotide
gcgctgcaaccaatagtcttcag

SEQ ID NO: 344 - DNA, 23 bp, Artificial Sequence
  Description of Artificial Sequence Synthetic oligonucleotide
gcgctgtaaccaatagtcttcgg

SEQ ID NO: 345 - DNA, 23 bp, Artificial Sequence
  Description of Artificial Sequence Synthetic oligonucleotide
gcgctgcaaccaatagtcttcag

SEQ ID NO: 346 - DNA, 23 bp, Artificial Sequence
  Description of Artificial Sequence Synthetic oligonucleotide
gcgctgcaaccaatagtcttcag

SEQ ID NO: 347 - DNA, 23 bp, Artificial Sequence
  Description of Artificial Sequence Synthetic oligonucleotide
gcgctgtaaccaatagtcttcgg

(19) United States
(12) Patent Application Publication
(10) Pub. No.: US 2019/0330643 A1
(43) Pub. Date: Oct. 31, 2019

(54) ENGINEERING OF BACTERIOPHAGES BY GENOME EDITING USING THE CRISPR-CAS9 SYSTEM

(71) Applicant: The Catholic University of America, Washington, DC (US)

(72) Inventors: Venigalla B. Rao, Silver Spring, MD (US); Pan Tao, Silver Spring, MD (US)

(21) Appl. No.: 16/355,932

(22) Filed: Mar. 18, 2019

Related U.S. Application Data
(60) Provisional application No. 62/662,272, filed on Apr. 25, 2018.

(51) Int. Cl. C12N 15/70; C12N 15/11; C12N 9/22

(52) U.S. Cl. CPC C12N 15/70; C12N 15/11; C12N 2795/10121; C12N 2310/20; C12N 9/22

(57) ABSTRACT

Embodiments of the invention provide systems, methods, and kits for CRISPR-based editing of DNA targets by a CRISPR-associated (Cas) enzyme. The systems include a bacterial host cell adapted to produce an engineered bacteriophage comprising a Cas protein and guide RNA that do not naturally occur together, i.e. they are engineered to occur together, as well as a DNA repair template comprising a donor DNA having a desired mutation. The guide RNA comprises a trans-activating crRNA and a guide sequence complementary to a target protospacer in a bacteriophage genome. A wild-type bacteriophage or a glucosylhydroxymethyl cytosine (ghmC)-unmodified mutant bacteriophage may be delivered into a disclosed bacterial host cell to create recombinants of bacteriophage having the desired mutation provided by the donor DNA.

### CROSS-REFERENCE TO RELATED APPLICATIONS

This application claims benefit of priority to U.S. Provisional Patent Application No. 62/662,272 to Rao and Tao, entitled “ENGINEERING OF BACTERIOPHAGES BY GENOME EDITING USING THE CRISPR-CAS9 SYSTEM,” filed Apr. 25, 2018. The entire contents and disclosures of the patent application are incorporated herein by reference in its entirety.

This application also makes reference to the following U.S. patents and U.S. patent applications: U.S. patent application Ser. No. 14/322,097, filed on Jul. 2, 2014, entitled “Protein and Nucleic Acid Delivery Vehicles, Components and Mechanisms Thereof,” now U.S. Pat. No. 9,523,101, which claims benefit of priority to U.S. patent application Ser. No. 13/082,466 to Rao, entitled “Protein and Nucleic Acid Delivery Vehicle, Components and Mechanisms Thereof” filed Apr. 8, 2011, now U.S. Pat. No. 8,802,418; and U.S. Provisional Patent Application No. 61/322,334 entitled a “A Promiscuous DNA Packaging Machine from Bacteriophage T4,” filed on Apr. 9, 2010; U.S. patent application Ser. No. 13/796,263, filed on Mar. 12, 2013, entitled “Protein and Nucleic Acid Delivery Vehicles, Components and Mechanisms Thereof,” now U.S. Pat. No. 9,365,867, which is a divisional application of U.S. patent application Ser. No. 13/082,466, filed Apr. 8, 2011; U.S. patent application Ser. No. 14/320,731, filed on Jul. 1, 2014, which claims benefit of priority to U.S. Provisional Patent Application No. 61/845,487 to Rao and Tao, entitled “Mutated and Bacteriophage T4 Nanoparticle Arrayed F1-V Immunogens from Yersinia Pestis as Next Generation Plague Vaccines,” filed Jul. 12, 2013; U.S. patent application Ser. No. 14/337,545, filed on Jul. 22, 2014, entitled “In Vitro and In Vivo Delivery of Genes and Proteins Using the Bacteriophage T4 DNA Packaging Machine,” now U.S. Pat. No. 9,187,765, which is a divisional of application Ser. No. 14/096,238 filed Dec. 4, 2013, now U.S. Pat. No. 9,163,262, which claims benefit of priority to U.S. Provisional Patent Application No. 61/774,895 filed Mar. 8, 2013, entitled “In Vitro and In Vivo Delivery of Genes and Proteins Using the Bacteriophage T4 DNA Packaging Machine”; U.S. patent application Ser. No. 11/015,294, filed Dec. 17, 2004, entitled “Methods and Compositions Comprising Bacteriophage Nanoparticles,” now U.S. Pat. No. 8,685,694, which claims priority to U.S. Provisional Application Ser. No. 60/530,527, filed Dec. 17, 2003, entitled “Methods and Compositions Comprising Bacteriophage Nanoparticles”; U.S. application Ser. No. 12/039,803, filed Feb. 29, 2008, entitled “T4 bacteriophage bound to a substrate,” now U.S. now U.S. Pat. No. 8,148,130, issued Apr. 3, 2012, which claims benefit of the U.S. Provisional Patent Application No. 60/904,168, filed Mar. 1, 2007, entitled “Liposome-Bacteriophage Complex as Vaccine Adjuvant.”

### GOVERNMENT INTEREST STATEMENT

This invention was made with the United States government support under NIAID/NIH Grant Nos. AI111538 and AI081726, awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Feb. 27, 2019, is named 109007-530002 SL.txt and is 80,933 bytes in size.

### BACKGROUND

### Field of the Invention

The present invention generally relates to systems, methods, and compositions for engineering of bacteriophages, and more particularly, to engineering of bacteriophages by genome editing using the CRISPR-Cas9 system.

### Background of Related Art

Bacteriophages inhabit all oceans, seas, rivers and waters on Earth, and probably constitute the largest proportion of the biomass on the planet (1). A large fraction of these phages are tailed, containing an icosahedral head (capsid) that houses a linear dsDNA genome and a tail that delivers the genome into a host bacterial cell (1, 2). However, very few phage genomes have been well-characterized, the tailed phage T4 genome being one of them. Even in T4, much of the genome remained uncharacterized. The classical genetic strategies are tedious, compounded by genome modifications such as cytosine hydroxylmethylation and glucosylation which makes T4 DNA resistant to most restriction endonucleases.

### SUMMARY

According to a broad aspect, the present invention provides an engineered system for editing a bacteriophage genome comprising a bacterial host cell adapted to produce an engineered bacteriophage using a Clustered Regularly Interspaced Short Palindromic Repeat (CRISPR)-CRISPR associated protein (Cas) (CRISPR-Cas). The bacterial host cell comprises a first nucleic acid sequence encoding a Cas protein, and a second nucleic acid sequence encoding a guide RNA (gRNA) comprising a trans-activating RNA (tracrRNA) and a CRISPR (cr) RNA containing guide sequence complementary to a target DNA sequence in a bacteriophage genome. The first nucleic acid sequence and the second nucleic acid sequence are operably linked to a same regulatory element or different regulatory elements operable in the bacterial host cell, on same or different vectors, whereby the Cas protein and the gRNA being expressed and forming a CRISPR-Cas complex in the bacterial host cell. It should be appreciated that the Cas protein and the gRNA do not naturally occur together, i.e. they are engineered to occur together in a recombinant plasmid.

According to another broad aspect, the present invention provides an engineered system for editing a bacteriophage genome comprising a bacterial host cell adapted to produce an engineered bacteriophage using CRISPR-Cas or similar technology. The bacterial host cell comprises a first nucleic acid sequence encoding a Cas9 protein, and at least one nucleic acid sequence encoding at least one guide RNA (gRNA). The at least one gRNA comprises a trans-activating crRNA (tracrRNA) and two or more guide sequences respectively complementary to two or more target DNA sequences in a bacteriophage genome. The first nucleic acid sequence encoding the Cas9 protein and the at least one nucleic acid sequence encoding the at least one guide RNA are operably linked to a same or different regulatory elements operable in the bacterial host cell, on same or different vectors, whereby the Cas9 protein and the at least one gRNA being expressed and forming at least one CRISPR-Cas complex in the bacterial host cell. It should be appreciated that the Cas protein and the at least one gRNA do not naturally occur together, i.e. they are engineered to occur together.

According to another broad aspect, the present invention provides a kit for editing a bacteriophage genome. The kit comprises one or more vectors containing a first nucleic acid sequence encoding a Cas9 protein and at least one nucleic acid sequence encoding at least one guide RNA (gRNA). The at least one guide RNA comprises a trans-activating crRNA (tracrRNA) and one or more guide sequences respectively complementary to one or more target DNA sequences in a bacteriophage genome. The first nucleic acid sequence and the at least one nucleic acid sequence encoding the at least one guide RNA are operably linked to a same regulatory element or different regulatory elements operable in a bacterial host cell, thereby allowing the Cas9 protein and the at least one gRNA to be expressed in the bacterial host cell. It should be appreciated that the Cas protein and the at least one gRNA do not naturally occur together, i.e. they are engineered to occur together.

According to another broad aspect, the present invention provides a method for editing a bacteriophage genome. The method comprises introducing a bacteriophage into a bacterial host cell containing a CRISPR-Cas spacer vector and a DNA repair template. The bacteriophage has a genome including one or more target DNA sequences. The CRISPR-Cas spacer vector comprises a first nucleic acid sequence encoding a Cas9 protein and at least one nucleic acid sequence encoding at least one guide RNA (gRNA). The at least one guide RNA comprises a trans-activating crRNA (tracrRNA) and one or more guide sequences respectively complementary to one or more target DNA sequences in a bacteriophage genome. The first nucleic acid sequence and the at least one nucleic acid sequence are operably linked to a regulatory element operable in the bacterial host cell. The Cas9 protein and the at least one gRNA are then expressed and form at least one CRISPR-Cas complex in the bacterial host cell. It should be appreciated that the Cas protein and the at least one gRNA do not naturally occur together, i.e. they are engineered to occur together. The at least one gRNA targets the one or more target DNA sequences in the bacteriophage genome and the Cas9 protein cleaves the bacteriophage genome. One or more double-strand breaks are generated in the one or more target DNA sequences. The DNA repair template includes a donor DNA sequence flanked by DNA segments homologous to end sequences of one of the one or more double-strand breaks. The donor DNA sequence includes at least one mutation to the bacteriophage genome, whereby the bacteriophage genome being altered after the donor DNA sequence is inserted into one of the one or more double-strand breaks through homology directed repair.

According to a fifth broad aspect, the present invention provides a method of determining an essentiality of a target gene of a bacteriophage. The method including introducing a null mutation to a target gene of a bacteriophage genome by the method of claim 44 using a DNA repair template comprising the null mutation, causing the target gene to fail to be translated into a function protein product; and performing a plaque assay for infection of bacterial host cells with bacteriophage having the null mutation and with wild type bacteriophage respectively. The target gene is determined to be nonessential if plaque formation for infection of bacterial host cells with bacteriophage that has the null mutation is similar to plaque formation for infection of bacterial host cells with wild type bacteriophage.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIGS. 1A-1E illustrate an experimental scheme for testing the effect of CRISPR-Cas on phage T4 infection according to one embodiment of the present invention.

FIGS. 2A and 2B illustrate restriction of phage T4 infection by CRISPR-Cas according to one embodiment of the present invention.

FIGS. 3A and 3B illustrate an alignment of CRISPR-Cas escape mutant sequences according to one embodiment of the present invention. FIGS. 3A and 3B disclose SEQ ID NOS 30-71, respectively, in order of appearance.

FIGS. 4A-4F illustrate an exemplary genome editing of phage T4 using CRISPR-Cas9 according to one embodiment of the present invention. FIG. 4F discloses SEQ ID NOS 72-75, respectively, in order of appearance.

FIGS. 5A-5C illustrate the effect of the length of the homologous arms on editing efficiency according to one embodiment of the present invention. FIG. 5A is a schematic of the donor templates with different lengths of homologous arms flanking the amber mutation site. FIG. 5A is an image showing plating of the phage lysates from the donor DNA templates shown in FIG. 5A. FIG. 5C is a graph showing the number of recombinant plaques produced using donor templates of different lengths.

FIGS. 6A-6C illustrate genome editing using two adjacent spacers according to one embodiments of the present invention. FIG. 6C discloses SEQ ID NOS 76-79, respectively, in order of appearance.

FIGS. 7A-7D illustrate the phage T4 rnlB being a nonessential gene.

FIG. 8A illustrates generation of a g20 amber mutant by CRISPR-Cas editing according to one embodiment of the present invention. FIG. 8A discloses SEQ ID NOS 80-82, respectively, in order of appearance.

FIG. 8B is a table showing the exemplary primers used for donor DNA constructions according to one embodiment of the present invention. FIG. 8B discloses SEQ ID NOS 83-100, respectively, in order of appearance.

FIGS. 9A-9E illustrate the evolution of phage T4 genome under CRISPR-Cas9 pressure. FIG. 9A is an experimental scheme for testing the effect of CRISPR-Cas on phage T4 infection according to one embodiment. FIGS. 9B and 9C illustrate plating efficiencies of high restriction spacers 20-1070 and 23-2, and low restriction spacers 20-995 and 23-1490. FIGS. 9B and 9C disclose SEQ ID NOS 101-108, respectively, in order of appearance. FIGS. 9D and 9E illustrate the alignment of sequences corresponding to single plaques produced from infection of various spacer-expressing E. coli. FIGS. 9D and 9E disclose SEQ ID NOS 109-152, respectively, in order of appearance.

FIGS. 10A, 10B, and 10C illustrate a model for CRISPR-Cas9 driven evolution of phage T4 genome according to one embodiment of the present invention.

FIGS. 11A-11F illustrate selection of CEMs in the portal protein gene according to one embodiment of the present invention. FIGS. 11A, 11C, 11E, and 11F disclose SEQ ID NOS 153-218, respectively, in order of appearance.

FIGS. 12A and 12B illustrate selection of CEMs in the major capsid protein gene according to one embodiment of the present invention. FIG. 12B discloses SEQ ID NOS 219-249, respectively, in order of appearance.

FIGS. 13A-13G illustrate the characteristics of the CEMs obtained from gene 20 spacers according to one embodiment of the present invention. FIG. 13A discloses SEQ ID NOS 250-261, respectively, in order of appearance.

FIGS. 14A-14G illustrate the characteristics of the CEMs obtained from gene 23 spacers according to one embodiment of the present invention. FIG. 14A discloses SEQ ID NOS 262-273, respectively, in order of appearance.

FIGS. 15A, 15B, 15C, and 15D are lists of all possible single mutations in each spacer that retain the reading frame according to one embodiment of the present invention. FIGS. 15A, 15B, 15C, and 15D disclose SEQ ID NOS 274-321, respectively, in order of appearance.

FIGS. 16A,16B, and 16C illustrate a relative fitness of the CEMs that escaped Cas9 cleavage of the protospacer 20-995 of the portal protein gene according to one embodiment of the present invention. FIGS. 16B and 16C disclose SEQ ID NOS 322-347, respectively, in order of appearance.

FIG. 17 illustrates the temperature sensitivity of the CEMs containing amino acid changes according to one embodiment.

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

For purposes of the present invention, it should be noted that the singular forms, “a,” “an” and “the” include reference to the plural unless the context as herein presented clearly indicates otherwise.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purpose of the present invention, the term “adjacent” refers to “next to” or “adjoining something else.”

For purposes of the present invention, the term “bind,” the term “binding” and the term “bound” refers to any type of chemical or physical binding, which includes but is not limited to covalent binding, hydrogen binding, electrostatic binding, biological tethers, transmembrane attachment, cell surface attachment and expression.

For purposes of the present invention, the term “cleavage” refers to breaking of a chemical bond in a nucleic acid molecule to separate or divide a nucleic acid molecule into two or more portions.

For purposes of the present invention, the term “capsid” and the term “capsid shell” refers to a protein shell of a virus comprising several structural subunits of proteins. The capsid encloses the nucleic acids of the virus. Capsids are broadly classified according to their structures. The majority of viruses have capsids with either helical or icosahedral structures.

For purposes of the present invention, the terms “prehead,” “prohead” or “procapsid,” “partial head” or “partially filled head,” “full head” and “phage head” in singular or plural form, refer to different stages of maturity of the viral capsid shell. “Prehead” refers to a capsid shell of precise dimensions or an isometric capsid that is initially assembled, often with a single type of protein subunit polymerizing around a protein scaffold. When the protein scaffolding is removed, creating an empty space inside the capsid shell, the structure is referred to as a prohead or a procapsid.

For purposes of the present invention, the tern Partial head, full head and phage head all refer to capsids that reach a stage of maturation that makes them larger, more stable particles associated with DNA. The term “partial head” refers to a mature capsid shell that either has only a portion of DNA packaged into it or it may refer to a mature capsid shell that was once packed full with DNA and then the DNA releases from the shell to leave only a small portion of DNA behind. The term “full head” refers to a mature capsid shell that is fully packed with DNA. Full heads can pack up to 105% of the bacteriophage genome. This is about 165-170 kb for T4 bacteriophages. Similarly, capsids of other viruses can also be packaged to accommodate more than their genomic volume. The capsid may or may not be enveloped. The maturation process of capsids in bacteriophages like HK97 is described, for example, in Lata et al., 2000 (Reference 42). GP23 is a capsid protein that self-associates to form hexamers, building most of the capsid in association with pentons made of the capsid vertex protein and one dodecamer of the portal protein. The major capsid protein self-associates to form 160 hexamers, building most of the T=13 laevo capsid. Folding of major capsid protein requires the assistance of two chaperones, the host chaperone groL acting with the phage encoded gp23-specific chaperone, gp31. The capsid also contains two nonessential outer capsid proteins, Hoc and Soc, which decorate the capsid surface. Through binding to adjacent gp23 subunits, Soc reinforces the capsid structure.

For purposes of the present invention, the term “complementary” refers to the ability of a nucleic acid to form hydrogen bond(s) with another nucleic acid sequence by either traditional Watson-Crick or other non-traditional types. As to DNA and RNA base pair complementarity, complementarity is achieved by distinct interactions between nucleobases: adenine, thymine (uracil in RNA), guanine and cytosine. Adenine and guanine are purines, while thymine, cytosine and uracil are pyrimidines. Purines are larger than pyrimidines. Both types of molecules complement each other and can only base pair with the opposing type of nucleobase. In nucleic acid, nucleobases are held together by hydrogen bonding, which only works efficiently between adenine and thymine and between guanine and cytosine. The base complement A=T shares two hydrogen bonds, while the base pair GiC has three hydrogen bonds. All other configurations between nucleobases would hinder double helix formation. DNA strands are oriented in opposite directions, they are said to be antiparallel. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

For purposes of the present invention, the term “comprising”, the term “having,” the term “including,” and variations of these words are intended to be open-ended and mean that there may be additional elements other than the listed elements.

For purposes of the present invention, the term “constitutively express” refers to the consistent synthesis of a protein. “Constitutively express” is contrary to “inducible expression” which depends on promoters that respond to the induction conditions.

For purposes of the present invention, the term “expression cassette” refers to a part of a vector DNA used for cloning and transformation. In each successful transformation, the expression cassette directs the cell's machinery to make RNA and protein. Some expression cassettes are designed for modular cloning of protein-encoding sequences so that the same cassette can easily be altered to make different proteins. Expression cassettes may also refer to a recombinantly produced nucleic acid molecule that is capable of expressing a genetic sequence in a cell. An expression cassette typically includes a regulatory region such as a promoter, (allowing transcription initiation), and a sequence encoding one or more proteins or RNAs. Optionally, the expression cassette may include transcriptional enhancers, non-coding sequences, splicing signals, transcription termination signals, and polyadenylation signals. The sequences controlling the expression of the gene, i.e. its transcription and the translation of the transcription product, are commonly referred to as regulatory unit. Most parts of the regulatory unit are located upstream of coding sequence of the heterologous gene and are operably linked thereto. The expression cassette may also contain a downstream 3′ untranslated region comprising a polyadenylation site. The regulatory unit of the invention is either directly linked to the gene to be expressed, i.e. transcription unit, or is separated therefrom by intervening DNA such as for example by the 5′-untranslated region of the heterologous gene. Preferably the expression cassette is flanked by one or more suitable restriction sites in order to enable the insertion of the expression cassette into a vector and/or its excision from a vector. Thus, the expression cassette according to the present invention can be used for the construction of an expression vector, in particular a mammalian expression vector.

For purposes of the present invention, the term “expression vector,” otherwise known as an expression construct, refers to a plasmid or virus designed for protein expression in cells. The vector is used to introduce a specific gene into a target cell, and can commandeer the cell's mechanism for protein synthesis to produce the protein encoded by the gene. The plasmid is engineered to contain regulatory sequences that act as enhancer and promoter regions and lead to efficient transcription of the gene carried on the expression vector. The goal of a well-designed expression vector is the production of significant amount of stable messenger RNA, and therefore proteins.

For purpose of the present invention, the term “flank” refers to be situated on a side of a polynucleotide sequence or an amino acid sequence.

For purposes of the present invention, the term “fragment” of a molecule such as a protein or a nucleic acid refers to a portion of an amino acid sequence of the protein or a portion of a nucleotide sequence of the nucleic acid.

For purpose of the present invention, the term “fuse” refers to join together physically, or to make things join together and become a single thing.

For purposes of the present invention, the term “gene” refers to a nucleic acid (e.g., DNA or RNA) sequence that comprises coding sequences necessary for the production of an RNA or a polypeptide or its precursor. The term “portion,” when used in reference to a gene, refers to fragments of that gene. The fragments may range in size from a few nucleotides to the entire gene sequence minus one nucleotide.

For purposes of the present invention, the term “gRNA targeting sequence” is a nucleotide sequence about 20 nts that precede the PAM sequence in a genomic DNA. In a CRISPR-Cas system, this sequence is cloned into a gRNA expression plasmid but does not include the PAM sequence or the gRNA scaffold sequence.

For purposes of the present invention, the term “guide RNA” or “gRNA,” as used in CRISPR Cas9 system, refers to RNAs that is a component of the CRISPR Cas9 system and comprises tracrRNA, crRNA, and a guide sequence that is an about 20 nucleotide sequence at the 5′ end of the gRNA. The “guide sequence” specifies the target side and may be used interchangeably with the terms “guide” or “spacer.” In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. A desired target sequence must immediately precede a 5′-NGG protospacer adjacent motif (PAM). The PAM sequence is not a part of the 20 base pair gRNA sequence, however, its presence in the genomic DNA is essential for CRISPR Cas9 genome editing. The term “tracr mate sequence” or “tracrRNA” may be used interchangeably with the term “direct repeat(s).”

For purposes of the present invention, the term “gRNA scaffold sequence” refers to the sequence within a gRNA that is responsible for Cas9 binding. It does not include a spacer/targeting sequence that is used to guide Cas9 to a target DNA sequence.

For purposes of the present invention, the term “homologous arm” or the term “homology arm,” when being used in making precise modifications using homology directed repair (HDR), interchangeably refers to a homologous segment or fragment of nucleotide sequence immediately upstream or downstream of a target DNA sequence. A homologous arm may be 5′ or 3′ homologous arm. A homologous segment may also be called a left homologous arm or aright homologous arm and flanks a desired edit in a DNA repair template.

For purposes of the present invention, the term “host cell” and the term “host” refer to 1) a cell that harbors foreign molecules, viruses, etc.; 2) a cell that has been introduced with DNA or RNA, such as a bacterial cell acting as a host cell for the DNA isolated from a bacteriophage.

For purposes of the present invention, the term “hybridization” refers to the process of forming a double stranded nucleic acid from joining two complementary strands of DNA (or RNA) (as in nucleic acid hybridization). A sequence capable of hybridizing with a given sequence is referred to as the “complement” of the given sequence. Particularly, hybridization is a technique in which molecules of single-stranded deoxyribonucleic acid (DNA) or ribonucleic acid (RNA) are bound to complementary sequences of either single-stranded DNA or RNA. Complementary base pairs are adenine (A) with thymine (T) or uracil (U) and vice versa, and guanine (G) with cytosine (C) and vice versa. Although the DNA double helix is relatively stable at body temperatures, high temperatures can split, or “melt,” the double helix into single, complementary strands. After disrupting the double helix in this way, lowering the temperature then causes the single-stranded DNA to base-pair, or anneal, to other single strands that have complementary sequences. Single-stranded DNA can hybridize to either single-stranded DNA or single-stranded RNA. Two complementary single-stranded DNA molecules can reform the double helix after annealing. In DNA-RNA hybridization, the RNA base uracil pairs with adenine in DNA.

For purposes of the present invention, the term “immune response” refers to a specific response of the immune system of an animal to antigen or immunogen. Immune response may include the production of antibodies and cellular immunity.

For purposes of the present invention, the term “incorporate” refers to insert a fragment of a first nucleic acid into a fragment of a second nucleic acid.

For purposes of the present invention, the term “modified” and the term “mutant” when made in reference to a gene or to a gene product refer, respectively, to a gene or to a gene product which displays modifications in sequence and/or functional properties (i.e., altered characteristics) when compared to the wild-type gene or gene product.

For purposes of the present invention, the term “mutation” refers to a change in the polypeptide sequence of a protein or in the nucleic acid sequence.

For purposes of the present invention, the term “neck protein” and the term “tail protein” refers to proteins that are involved in the assembly of any part of the necks or tails of a virus particle, in particular bacteriophages. Tailed bacteriophages belong to the order Caudovirales and include three families: The Siphoviridae have long flexible tails and constitute the majority of the tailed viruses. Myoviridae have long rigid tails and are fully characterized by the tail sheath that contracts upon phage attachment to bacterial host. The smallest family of tailed viruses are podoviruses (phage with short, leg-like tails). For example, in T4 bacteriophage gp10 associates with gp11 to forms the tail pins of the baseplate. Tail-pin assembly is the first step of tail assembly. The tail of bacteriophage T4 consists of a contractile sheath surrounding a rigid tube and terminating in a multiprotein baseplate, to which the long and short tail fibers of the phage are attached. Once the heads are packaged with DNA, the proteins gp13, gp14 and gp15 assemble into a neck that seals of the packaged heads, with gp13 protein directly interacting with the portal protein gp20 following DNA packaging and gp14 and gp15 then assembling on the gp13 platform. Neck and tail proteins in T4 bacteriophage may include but are not limited to proteins gp6, gp25, gp53, gp8, gp10, gp11, gp7, gp29, gp27, gp5, gp28, gp12, gp9, gp48, gp54, gp3, gp18, gp19, gp13, gp14, gp15 and gp63. Aspects of the neck and tail assembly proteins in T4 bacteriophage and other viruses are described further, for example, in Rossmann et al., 2004 (Reference 41).

For purposes of the present invention, the term “non-naturally occurring” or “isolated” refers to the component of interest being at least substantially free from at least one other component with which it is naturally associated in nature and as found in nature. The term “isolated,” when used in relation to a nucleic acid, as in “an isolated oligonucleotide,” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

For purposes of the present invention, the term “mutation” refers to a change in the polypeptide sequence of a protein or in the nucleic acid sequence.

For purposes of the present invention, the terms “nucleic acid,” “polynucleotide,” “nucleotide sequence,” “nucleic acid,” and “oligonucleotide,” as used interchangeably herein, refer to polymers of nucleotides of any length, and include DNA and RNA. The nucleic acid bases that form nucleic acid molecules can be the bases A, C, G, T and U, as well as derivatives thereof. Derivatives of these bases are well known in the art. The term should be understood to include, as equivalents, analogs of either DNA or RNA made from nucleotide analogs. The term as used herein also encompasses cDNA, that is complementary, or copy, DNA produced from an RNA template, for example by the action of reverse transcriptase. A polynucleotide may comprise modified nucleotides, such as methylated nucleotides and their analogs.

For purposes of the present invention, the term “operably linked,” the term “operably associated,” and the term “functionally linked” are used interchangeably and refer to a functional relationship between two or more DNA segment. Particularly, “operably linked” may refer to place a first nucleic acid sequence in a functional relationship with the second nucleic acid sequence. For example, a promoter/enhancer sequence, including any combination of cis-acting transcriptional control elements is operably associated or operably linked to a coding sequence if the promoter/enhancer sequence affects the transcription or expression of the coding sequence in an appropriate host cell or other expression system. Promoter regulatory sequences that are operably linked to the transcribed gene sequence are physically contiguous to the transcribed sequence. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

For purposes of the present invention, the term “packaging machine” refers to the complete packaging unit including the compartment, the motor and the component or any other attachment mechanism that connects the motor to the compartment. For example, the T4 packaging machine comprises the shell (the procapsid made primarily of gp23), the vertex portal protein (dodecameric gp20) and the gp17 packaging motor. A packaging machine may be one described in patents and applications listed in the section of cross-reference to related application.

For purposes of the present invention, the term “packaging motor” refers to a molecular motor or a molecular machine that is capable of using chemical energy to drive the mechanical translocation of a nucleic acid and package the nucleic acid into a compartment. For example, the packaging motor in T4 bacteriophage uses the energy of ATP hydrolysis to translocate and package DNA into the capsid shell. The packaging motor may be a protein complex comprising one or more protein subunits and have enzymatic activities that help package nucleic acids, which include, but are not limited to ATPase, nuclease and translocase. For example, T4 bacteriophage packaging motor refers to a large terminase protein, the pentameric gene product (gp)17. The term “packaging motor” may also be considered to encompass additional proteins that regulate or enhance the activity of the actual motor. For example, the T4 packaging motor may also include a small terminase protein gp 16. The T4 DNA packaging motor is further described in, for example, Sun et al., 2008.

For purposes of the present invention, the term “phage therapy” or “viral phage therapy” refers to a therapeutic use of bacteriophages to treat pathogenic bacterial infections. Phage therapy has many potential applications in human medicine as well as dentistry, veterinary science, and agriculture. Bacteriophages are much more specific than antibiotics. Phages are viruses that only infect bacteria. They are typically harmless not only to the host organism, but also to other beneficial bacteria, reducing the chances of opportunistic infections. They have a high therapeutic index, that is, phage therapy would be expected to give rise to few side effects. Because phages replicate in vivo (in cells of living organism), a smaller effective dose may be used. Bacteriophage treatment offers a possible alternative to conventional antibiotic treatments for bacterial infection. Bacteriophages are very specific, targeting only one or a few strains of bacteria. Traditional antibiotics have more wide-ranging effect, killing both harmful bacteria and useful bacteria such as those facilitating food digestion. The species and strain specificity of bacteriophages makes it unlikely that harmless or useful bacteria will be killed when fighting an infection.

For purposes of the present invention, the term “primer” refers to an oligonucleotide, whether occurring naturally as in a purified restriction digest or produced synthetically, that is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method. One with ordinary skill in the art of design of primers will recognize that a given primer need not hybridize with 100% complementarity to prime the synthesis of a complementary nucleic acid strand. Primer pair sequences may be a “best fit” amongst several aligned sequences, thus they need not be fully complementary to the hybridization region of any one of the sequences in the alignment. Moreover, a primer may hybridize over one or more segments such that intervening or adjacent segments are not involved in the hybridization event (e.g., for example, a loop structure or a hairpin structure). The primers may comprise at least 70%, at least 75%, at least 80%, at least 85%, at least 90%, at least 95% or at least 99% sequence identity with a target nucleic acid of interest. Thus, in some embodiments, an extent of variation of 70% to 100%, or any range falling within, of the sequence identity is possible relative to the specific primer sequences disclosed herein. To illustrate, determination of sequence identity is described in the following example: a primer 20 nucleobases in length which is identical to another 20 nucleobase primer having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer. Percent identity need not be a whole number, for example when a 28 consecutive nucleobase primer is completely identical to a 31 consecutive nucleobase primer (28/31=0.9032 or 90.3% identical).

For purposes of the present invention, the term “promoter” refers to a regulatory DNA sequence generally located upstream of a gene that mediates the initiation of transcription by directing RNA polymerase to bind to DNA and initiating RNA synthesis.

For purposes of the present invention, the term “polypeptide,” the term “peptide,” and the term “protein” are used interchangeably herein to refer to a polymer of amino acid residues. The terms encompass amino acid polymers in which one or more amino acid residues are artificial chemical mimetic of a corresponding naturally occurring amino acids, as well as to naturally occurring amino acid polymers and non-naturally occurring amino acid polymer.

For purposes of the present invention, the term “promoter” refers to a regulatory sequence that will determine in which cells and at what time a transgene is active. The promoter sequence normally contains the transcriptional start site as well as the transcription regulatory sequences. In addition, the promoter sequence also typically contains some extraneous sequence downstream of the transcriptional start. Synthetic promoters have also been designed for inducible gene expression and other specialized applications.

For purposes of the present invention, the term “protein domain” or “domain” refers to a distinct functional or structural unit in a protein. Usually, a protein domain is responsible for a particular function or interaction, contributing to the overall role of a protein. Domains may exist in a variety of biological contexts, where similar domains may be found in proteins with different functions.

For purposes of the present invention, the term“purified” refers to a component in a relatively pure state, e.g. at least about 90% pure, or at least about 95% pure, or at least about 98% pure. A purified component may be either a nucleic or an amino acid sequence that is removed from their natural environment, isolated or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the term “purified” or “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

For purposes of the present invention, the term “recombinant” refers to a genetic material formed by a genetic recombination process. A “recombinant protein is made through genetic engineering. A recombinant protein is coded by a DNA sequence created artificially. A recombinant protein is a protein that is coded by a recombinant nucleic acid sequence. A recombinant nucleic acid sequence has a sequence from two or more sources incorporated into a single molecule.

For purposes of the present invention, the term “regulatory region” or “regulatory element” refers to a segment of a nucleic acid molecule which is capable of increasing or decreasing the expression of specific genes within an organism. A regulatory sequence may include enhancer/silencer, operator, and promoter regions which regulate the transcription of the gene into an mRNA.

For purpose of the present invention, the term “restriction,” as used in context like “restriction of genome,” refers to cleavage of genome by Cas9 protein.

For purpose of the present invention, the term “subunit” refers to a separate polypeptide chain that makes a certain protein which is made up of two or more polypeptide chains joined together. In a protein molecule composed of more than one subunit, each subunit may form a stable folded structure by itself. The amino acid sequences of subunits of a protein may be identical, similar, or completely different.

For purposes of the present invention, the term “vector” and the term “suitable vector” refer to a nucleic acid molecule capable of transporting another nucleic acid to which it has been linked or incorporated. A vector (for example, a plasmid or virus) may incorporate a piece of a nucleic acid having a sequence encoding an antigenic polypeptide and any desired control sequences. A plasmid is a circular double stranded DNA loop into which additional DNA fragments or segments may be inserted, such as by standard molecular clonging techniques. The choice of the vector will typically depend on the compatibility of the vector with a host cell into which the vector is to be introduced. A vector may be an expression vector that brings about the expression of a piece of a nucleic acid. An expression vector is usually a plasmid or virus designed for gene expression in cells. For example, a lentiviral vector is a vector derived from (i.e., sharing nucleotides sequences unique to) to lentivirus. A vector may be used to introduce a specific gene into a target cell, and may commandeer the cell's mechanism for protein synthesis to produce the protein encoded by the gene. A specific gene introduced into a target cell may also commandeer the cells' mechanism for producing RNA having a sequence that is complementary to the sequence of the specific gene. A plasmid may be engineered to contain regulatory sequences that act as enhancer and promoter regions and lead to efficient transcription of the gene carried on the expression vector.

For purposes of the present invention, the term “system” refers to a set of components, real or abstract, comprising a whole where each component interacts with or is related to at least one other component within the whole.

For purposes of the present invention, the term “variant” refers to one that exhibits variation from a type or norm, such as a variant strains that exhibits qualities that have a pattern deviating from what occurs in nature.

For purposes of the present invention, the term “vector” refers to any nucleic acid vector known in the art. Such vectors include, but are not limited to, plasmid vectors, cosmid vectors and bacteriophage vectors. For example, one class of vectors utilizes DNA elements which are derived from animal viruses such as animal papilloma virus, polyoma virus, adenovirus, vaccinia virus, baculovirus, retroviruses (RSV, MMTC or MoMLV), Semliki Forest virus or SV40 virus. The eukaryotic expression plasmid PPI4 and its derivatives are widely used in constructs described herein. However, the invention is not limited to derivatives of the PPI4 plasmid and may include other plasmids known to those skilled in the art. In accordance with the invention, numerous vector systems for expression of recombinant proteins may be employed. For example, one class of vectors utilizes DNA elements which are derived from animal viruses such as bovine papilloma virus, polyoma virus, adenovirus, vaccinia virus, baculovirus, retroviruses (RSV, MMTV or MoMLV), Semliki Forest virus or SV40 virus. Additionally, cells which have stably integrated the DNA into their chromosomes may be selected by introducing one or more markers which allow for the selection of transfected host cells. The marker may provide, for example, prototropy to an auxotrophic host, biocide (e.g., antibiotic) resistance, or resistance to heavy metals such as copper or the like. The selectable marker gene may be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals. The cDNA expression vectors incorporating such elements include those described by (Okayama and Berg, 1983).

purposes of the present invention, the term “animal” refers to mammal, reptile, avian and fish species.

For purposes of the present invention, the term “wild-type” and the term “native,” refer to a strain, gene, or characteristic which prevails among individuals in natural conditions, as distinct from an atypical mutant type. The term “wild-type” and the term “native,” when made in reference to a gene product, refers to a gene product that has the characteristics of a gene product isolated from a naturally occurring source. The term “naturally-occurring” as applied to an object refers to the fact that an object may be found in nature. A wild-type gene is frequently that gene which is most frequently observed in a population and is thus arbitrarily designated the “normal” or “wild-type” form of the gene.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques may be used for chemical syntheses, chemical analyses, pharmaceutical preparation, formulation, and delivery, and treatment of patients. Standard techniques may be used for recombinant DNA, oligonucleotide synthesis, and tissue culture and transformation (e.g., electroporation, lipofection). Reactions and purification techniques may be performed e.g., using kits of manufacturer's specifications or as commonly accomplished in the art or as described herein. The foregoing techniques and procedures may be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

### Description

While the invention is susceptible to various modifications and alternative forms, specific embodiment thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention.

Embodiments of the present disclosures provide methods, systems, and kits for engineering of bacteriophages by genome editing using the CRISPR-CAS9 system.

Bacteriophages (phages) and bacteria are the most abundant organisms on Earth (1, 47). Phages infect bacteria and often kill them by using the cell as a factory to manufacture hundreds of new viruses and dissolving the cellular envelope to release the progeny. A single viral genome delivered by a single phage is sufficient to take control of the entire cell and divert the resources to assemble viruses (19). Bacteria have evolved strategies to defend themselves against this onslaught by phages, such as the production of restriction endonucleases that may digest the phage genome (48). Phages in turn have evolved counter-defenses such as the modification of the genome making it resistant to nucleases (19, 27). Although the molecular mechanisms of many of these innate defensive strategies are well understood, how the bacteria and phages, despite this perpetual “arms race,” have evolved to dominate the Earth's biomass is still poorly understood.

Since their discovery in early 20th century, phages has served as extraordinary models to elucidate the basic mechanisms of life and to create new avenues for genetic engineering and phage therapies (1, 3-5). Felix d'Herelle, a French-Canadian scientist at Institute Pasteur and the co-discoverer of bacteriophages used cocktails of lytic phages to treat bacterial infections nearly a century ago (3, 5). However, phage therapy lags behind because of the discovery of small molecule antibiotics that provide greater breadth and potency (3, 5, 6). The emergence of multi-antibiotic resistant bacterial pathogens and their continuing spread in the population brought new urgency to develop phage-based therapies (6, 7). A striking example is the recent case in San Diego where an individual infected with the multi-drug resistant Acinetobacter baumannii went into coma for nearly two months but completely recovered after intravenous administration of a mixture of phages that infect and lyse this bacterium (8).

Phages have also emerged as powerful vaccine and gene therapy platforms to deliver genes and proteins into mammalian cells (9-11). One such platform using T4, a tailed phage belonging to the Myoviridae family has been developed (12-14). A unique feature of phage T4 is that its 120×86 nm size capsid (head) is decorated with two nonessential outer capsid proteins, Soc or small outer capsid protein (870 copies) and Hoc or highly antigenic outer capsid protein (155 copies) (2, 15). Antigens fused to Soc or Hoc, up to 1,025 per head, may be displayed on the hoc⁻soc⁻ T4 capsid with high affinity and exquisite specificity (12, 13, 16, 17). Such nanoparticles may elicit robust immune responses and confer protection against deadly infections such as anthrax and plague (12, 17). Furthermore, the interior of the capsid may be filled with foreign DNA, up to ˜170-kb, either a single long DNA molecule or multiple short plasmid DNAs (13). These nanoparticles could be targeted to specific cells to deliver combinations of genes and proteins, which could eventually lead to human therapies against genetic and infectious diseases (13).

Considering the magnitude and diversity of phages, there is vast potential to harness their genomes for biomedical applications. However, other than a few phages that have been well characterized, very little has been done to unleash this potential, largely because it is tedious to manipulate phage genomes using the classical genetic strategies (18). For instance, even in the well-studied phage T4, of ˜300 potential genes in its 170-kb genome, nearly 130 of them remained uncharacterized (19, 20). The ˜100 or so nonessential genes are distributed throughout the genome and the genome is circularly permuted with no unique ends, making it extremely difficult to engineer T4 as a cloning or mammalian delivery vector (19, 20).

CRISPR (clustered regularly interspaced short palindromic repeat)-Cas (CRISPR-associated) is a remarkable adaptive defense system recently discovered in bacteria and archaea (21, 7). When a phage infects a bacterium, it incorporates short 20-40 bp segments of phage genome (“spacers”) into a CRISPR array present in the bacterial genome. In the surviving bacteria, these spacers are expressed as CRISPR RNAs (crRNAs) and provide a surveillance mechanism for the descendant cells (21, 49). When the cells are infected by the same phage, the crRNAs guide the CRISPR-Cas system to the respective spacer sequence in the phage genome (protospacer) and cleaves it (49). The bacterial genome is protected because the spacers in its CRISPR array lack additional recognition elements such as the PAM (Protospacer Adjacent Motif) sequence. The cleaved phage genome is cannibalized, potentially to acquire additional spacers, and no longer able to support a productive phage infection.

The type II CRISPR-Cas9 from Streptococcus pyogenes is the simplest and the best studied bacterial adaptive immune system (21). It consists of three basic components; crRNA derived from the spacer sequences incorporated into the CRISPR array, tracrRNA that is common to all spacers, and Cas9 nuclease, together assembling as a CRISPR-Cas9 complex. Guided by spacer-specific crRNA, the complex recognizes a three nucleotide 5′-NGG-3′ PAM sequence plus the upstream complementary protospacer sequence in phage genome and makes a double-stranded DNA break in the protospacer sequence. The disrupted genome may be further degraded by nonspecific nucleases in the cell resulting in the inactivation of phage genome and loss of plaque forming ability.

Recently, Clustered Regularly Interspaced Short Palindromic Repeats (CRISPR)-Cas system was developed as an efficient tool for targeted genome editing in many organisms (21). CRISPR-Cas is an acquired immune system evolved in bacteria and archaea to counter the invasion of phages and foreign genetic elements such as plasmids. The type II CRISPR-Cas from Streptococcus pyogenes, which contains three components: crRNA derived from the unique spacer sequences present in the CRISPR region, tracrRNA that is common to all spacers, and Cas9 nuclease, is the most commonly used system for genome modification (21). When expressed in a cell, these components form a CRISPR-Cas9 complex and create a double-strand DNA break at a specific site in the genome (protospacer) that is complementary to the spacer sequence present in the crRNA. The break may then be repaired and rejoined, or recombined with a donor DNA using other DNA metabolizing enzymes present in the cell to generate mutants of interest (21).

The CRISPR-Cas9 system has been extensively exploited for targeted editing of mammalian genomes and to generate genetically modified cell lines and organisms (50). However, relatively little attention has been given to understand the basic biology of CRISPR-Cas and its role in host-virus relationships. The CRISPR-containing bacteria have the capability to essentially wipe out the susceptible phages, as documented by several studies (51). Rare CRISPR-escape mutant phages would no doubt survive but the bacteria may acquire additional spacer(s) from the resistant phage and become rapidly immune gaining an upper hand in this arms race (51). This would not only deplete phage populations but also impact bacterial evolution because horizontal gene transfer, a key driver of bacterial evolution, is largely dependent on productive phage infections(52). Hence, robust levels of phages must co-exist in order for both the bacteria and the phages to thrive (52).

Several anti-CRISPR mechanisms have been recently discovered in phages and in lysogenic bacteria containing integrated prophage genomes (26-54). These provide counter-defenses for phage survival by interfering with various steps of the CRISPR-Cas pathways and limiting the effectiveness of the CRISPR-mediated genome disruption. However, their role in phage and/or bacterial evolution is unknown.

Although this CRISPR-Cas system has been extensively used to modify mammalian genomes, surprisingly, there have been very few reports employing the CRISPR-Cas to engineer phage genomes (22-25). It is possible that the anti-CRISPR mechanisms evolved in phages may limit the application of CRISPR-Cas to edit phage genomes (26). The short time window of lytic phage life cycle, ˜20-30 minutes, might be another limitation. Furthermore, many of the phages have evolved genome modifications in defense of host restriction systems. Phage T4 genome is particularly notorious because its cytosines are modified by two modifications, 5-hydroxymethylation and glucosylation (19, 27, 28). Consequently, the glucosyl hydroxymethyl cytosine (ghmC)-genome of phage T4 is highly resistant to most restriction endonucleases (19, 27).

It is unclear whether the ghmC-modifications protect the genome against attack by the CRISPR-Cas type host defenses. Yaung et al. reported that three spacers utilized by the CRISPR-Cas9 system are functional against wild-type (WT) ghmC-modified T4 genome (28). On the other hand, Bryson et al. reported that the ghmC-modification makes the T4 genome resistant to CRISPR-Cas9 attack based on data from four spacers that prevented unmodified T4(C) mutant phage infection but not the ghmC-modified WT T4 infection (27).

Phage T4 is one of the most well characterized viruses. The atomic structures of essentially all the key components of the virus including the head, tail, fibers, and the DNA packaging machine have been determined (2, 35-40). Genetic and biochemical pathways were elucidated in 60's and 70's that revealed common principles of virus assembly (19). Combined with the unique features of the T4 outer capsid proteins Hoc and Soc and the promiscuous nature of the DNA packaging machine, a platform to deliver genes and proteins into mammalian cells has been developed (12, 13, 41). However, it has been difficult to engineer the T4 genome owing to its modified genome that is refractory to most restriction enzymes (19, 27). Lack of a clustered nonessential region in the genome that may be replaced with foreign DNA posed another barrier to use T4 as a cloning or protein/gene delivery vector (20). Overcoming such barriers would be essential to unleash the potential of T4 and other phages for biomedical applications. Our studies reported here demonstrate that some of these barriers could be overcome by CRISPR-Cas genome editing, which could potentially be extended to phages in general.

Previously, researchers used tedious strategies to engineer phage genomes. These include treatment with mutagenic reagents, amplification by polymerase chain reaction using mutant primers and so on. This requires laborious screening protocols to identify the desired mutant among a large amount of background. Often, the desired mutant is not found thereby requiring multiple rounds of mutagenesis. Embodiments of the present invention use the CRISPR-Cas genome editing process that is precisely directed to the site where mutations need to be created. In addition, by providing a donor DNA containing the desired mutations, the original DNA may be replaced by the donor DNA generating a recombinant phage that could be used in various phage therapy applications.

According to the embodiments disclosed herein, by using a large number of spacers spanning the T4 genome, embodiments of the present disclosure demonstrate that the ghmC-modified T4 genome is vulnerable to cleavage by the Cas9 nuclease. However, it is not as susceptible to Cas9 as the unmodified genome and the efficiency of restriction of phage infection varied greatly depending on the spacer used, in part explaining the differences in previous studies (27, 28). The modified genome, however, is less susceptible to Cas9 nuclease attack when compared to the unmodified genome. The efficiency of restriction of modified phage infection varies greatly in a spacer-dependent manner, which explains some of the previous contradictory results.

Accordingly, a strategy to edit either the unmodified or the ghmC-unmodified T4 genome by introducing point mutations, insertions, and deletions is developed. In an example, this editing strategy is applied to determine whether the RNA ligase II gene rnlB is essential for phage infection. A 387-bp deletion knock-out mutation in RNA ligase gene rnlB, including a 294-bp deletion in RNA ligase gene rnlB and its upstream region, produces viable plaques and similar burst size as the WT T4 genome, demonstrating that the rnlB function is not essential for phage infection under the laboratory conditions. This example demonstrates the usefulness of this editing strategy to determine the essentiality of a given gene. These results establish the first phage genome editing system in T4, for both the unmodified and ghmC-modified genomes, which is potentially extended to other phage genomes in nature to create useful recombinants for phage therapy applications.

Based on some unexpected findings, it is proposed that the CRISPR-Cas system might have evolved not merely to protect the bacterial host from phage infection but also, to potentially benefit the phage by allowing rapid evolution. As disclosed, the wild-type (WT) phage T4 genome modified by cytosine hydroxymethylation and glucosylation (ghmC-T4) is much less vulnerable to S. pyogenes CRISPR-Cas9 cleavage when compared to the T4(C) mutant phage containing the unmodified cytosine genome (55). In this system, the crRNA containing the spacer sequences that are complementary to the protospacers in the T4 genome adjacent to a PAM sequence, as well as the tracrRNA and Cas9 nuclease are expressed constitutively from a plasmid. Hence, the susceptibility of the T4 genome to CRISPR-Cas9 attack and the post-cleavage mechanisms that respond to a single double-stranded break introduced into the T4 genome could be examined. Surprisingly, the analyses reveal that the plaques generated from the WT ghmC-phage infections accumulated CRISPR-escape mutations (CEMs) at extraordinary rates. In fact, it is so rapid that about 5-10% of the first generation plaques contain, predominantly, the CEM phages, and essentially 100% of the plaques become CEM by the third generation. These results suggest that the CRISPR-Cas not only protects bacteria against phages but also, drives rapid phage evolution which in turn is essential for bacterial evolution. This double-edged role of CRISPR-Cas, and possibly other bacterial/phage defensive mechanisms, suggest that these systems could provide selective advantages to both bacteria and phages, not merely to one or the other, that are essential for co-evolution and ultimately, their dominance on the planet.

In general, embodiments of the present invention provide strategies, methods and novel systems for altering or modifying bacteriophage genome using a CRISPR-CAS system, such as the type-II CRISPR-Cas9 system. Altering or modifying bacteriophage genome includes altering or modifying expression of one or more gene products of a bacteriophage. In a genome editing strategy described herein, a CRISPR-Cas9 plasmid and a donor plasmid containing desired mutation(s) may be co-delivered into a host cell such as E. coli. Single and multiple point mutations, insertions and deletions may be introduced into both modified and unmodified phage genomes. As short as 50-bp homologous flanking arms may be sufficient to generate recombinants that may be selected under the pressure of CRISPR-Cas9 nuclease.

As discussed above, bacteriophages hold enormous potential to develop therapeutics to treat human diseases caused either by genetic defects or by infectious agents. Using the methods and systems disclosed herein, bacteriophages may be easily engineered by introducing mutations into the genome. The recombinant phages thus engineered may be used in various phage therapy applications in biotechnology and medicine.

It should be noted that bacteriophages share extended structural and functional similarities. Bacteriophages with icosahedral heads, dodecameric portal vertex, dsDNA genome, and a tail are the most abundant virus type. Double stranded DNA icosahedral bacteriophages follow common mechanisms of assembly, genome packaging, genome delivery, and infection. Bacteriophage T4 is one of the seven Escherichia coli phages (T1-T7, T for type). T4-like phages are a diverse group of lytic bacterial myoviruses that share genetic homologies and morphological similarities with the well-studied phage T4. Accordingly, the methods, systems, and kits disclosed herein are not limited to be applied to phage T4, but should be extended to any bacteriophage, including any types of phage T1-T7 and other double stranded DNA bacteriophages such as λ, P22, SPP1, and numerous others (71).

In one aspect, embodiments provide an engineered system for editing a bacteriophage genome, including modifying or altering one or more gene products of the bacteriophage. The engineered system comprises a bacterial host cell, such as an Escherichia coli (E. coli) bacterial cell, adapted to produce an engineered bacteriophage using CRISPR-Cas. In one embodiment, the bacterial host cell includes a first nucleic acid sequence encoding a Cas protein, such as type II CRISPR-associated nuclease enzyme Cas9 derived from Streptococcus pyogenes, and a second nucleic acid sequence encoding a guide RNA (gRNA) comprising a trans-activating crRNA (tracrRNA) and a guide sequence (crRNA) complementary to a target DNA sequence in a bacteriophage genome. The first nucleic acid sequence and the second nucleic acid sequence are operably linked to a same regulatory element or different regulatory elements operable in the bacterial host cell. The first nucleic acid sequence and the second nucleic acid sequence may locate in one vector or different vectors. In some embodiment, the vector or vectors are plasmids. Each vector includes an expression cassette to express the Cas protein and/or the guide RNA components. Nucleic acid sequences for Cas protein and/or the guide RNA components maybe controlled under one promoter. In some embodiment, the guide RNA is expressed as a single guide RNA in which the tracrRNA is connected to the guide sequence. In some embodiment, the Cas protein and the guide sequence are constitutively expressed under the control of the promoter. As a result, Cas protein and the gRNA are continuously produced in the bacterial host cell. The Cas protein and the gRNA form a CRISPR-Cas complex in the bacterial host cell. The CRISPR-Cas complex recognizes a PAM sequence, such as a three nucleotide 5′-NGG-3′, plus the upstream complementary protospacer sequence in phage genome and makes a double-stranded DNA break in the protospacer sequence. It should be noted that the Cas protein and the gRNA do not naturally occur together in the bacterial host cell, i.e. they are engineered to occur together.

Accordingly, spacers corresponding to protospacers preceding a PAM in a bacteriophage genome may be used to produce guide sequences to target corresponding target DNA sequence in a bacteriophage genome. In some embodiments, spacers listed in Table 1 are used as nucleic acid sequences to produce guide sequences as components in guide RNA to target a protospacer and guide Cas protein to cleave the genome of the bacteriophage at a specific site in the protospacer. Accordingly, a nucleotide sequence for producing a guide sequence (crRNA) may be a sequence of one of SEQ ID NOS 1-25.

TABLE 1
Spacer Sequences^{a}

| spacer | | | SEQ ID | | | |
|---|---|---|---|---|---|---|
| no. | gene | sequence (5′ to 3′) | NO: | C (%) | G(%) | CG (%) |
| 1 | 23-2 | aagaacttccaaccggtaat | 1 | 25 | 15 | 40 |
| 2 | 20-1070 | gcaatatggaagatattcgt | 2 | 10 | 25 | 35 |
| 3 | Mrh.2-24 | ataatatctaaatcttcatt | 3 | 15 | 0 | 15 |
| 4 | 39.1-151 | atgttctgggctgttcttta | 4 | 15 | 25 | 40 |
| 5 | dda.1-130 | gagcagtatcgattagcttt | 5 | 15 | 25 | 40 |
| 6 | segF496 | gatttcagaaggaacttcaa | 6 | 15 | 20 | 35 |
| 7 | rnlb270 | gttgtatcttatcaagtctt | 7 | 15 | 15 | 30 |
| 8 | srh-8 | ttctgaagaagatgcttgaa | 8 | 10 | 25 | 35 |
| 9 | dexA523 | cttccaaagggaactttaga | 9 | 20 | 20 | 40 |
| 10 | cef154 | tcacgaaatacaccataaat | 10 | 25 | 5 | 30 |
| 11 | 39-37 | gaacatatcaaaaagcgtag | 11 | 15 | 20 | 35 |
| 12 | 24.2-181 | ttacagaagaaattggtgat | 12 | 5 | 25 | 30 |
| 13 | 20-995 | gcgctgcaaccaatagtctt | 13 | 30 | 20 | 50 |
| 14 | 39-96 | gcgctttatgtttggtaaat | 14 | 10 | 25 | 35 |
| 15 | 24-38 | gattgagttgctattcgttg | 15 | 10 | 30 | 40 |
| 16 | e148 | gttagtaaatcctgccacac | 16 | 30 | 15 | 45 |
| 17 | segD36 | tattcgcaattggataaatg | 17 | 10 | 20 | 30 |
| 18 | uvsy263 | agcggataaggatgttttaa | 18 | 5 | 30 | 35 |
| 19 | 23-1490 | gaatagaaggcataccgctc | 19 | 25 | 25 | 50 |
| 20 | uvsy287 | tgatacctcgttgcagtatt | 20 | 20 | 20 | 40 |
| 21 | 56-136 | aatttcgagctggtcttcgg | 21 | 20 | 30 | 50 |
| 22 | 56-63 | tttcttccgcattcattcca | 22 | 35 | 5 | 40 |
| 23 | e44 | caacgtttagaactggcact | 23 | 25 | 20 | 45 |
| 24 | IP3-28 | ggcctttactacagaagctt | 24 | 25 | 20 | 45 |
| 25 | IP3-10 | accacgggctgcattagcaa | 25 | 30 | 25 | 55 |

^{a}Sequences of the spacers used in the current study. Five spacers (spacers 1-5), which showed similar EOP for both WT and T4(C) mutant infections, are highlighted in bold font.

For example, a nucleic acid sequence encoding the guide sequence may be a spacer of about 20 nucleotides (nt) from a gene encoding bacteriophage capsid protein, eg., gp23. In some embodiments, the nucleic acid sequence encoding the guide sequence may be a sequence set forth in SEQ ID NO: 1 or 19, which are derived from protospacer in gene for gp23. In some other embodiments, a nucleic acid sequence encoding the guide sequence comprises a spacer of about 20 nucleotides (nt) from a gene encoding a bacteriophage portal protein such as gp20. The nucleic acid sequence encoding the guide sequence may comprise a sequence set forth in SEQ ID NO: 2 or 13, which are derived from protospacer in gene for gp20.

In some embodiments, in the above described system, the bacteriophage that is bacteriophage T4. In some embodiment, the above bacteriophage is a glucosylhydroxymethyl cytosine (ghmC)-unmodified mutant phage. In one embodiment, the ghmC-unmodified mutant phage contains an amber mutation in gene 42 that codes for deoxycytidine monophosphate hydroxymethylase (g42) and an amber mutation in gene 56 that codes for deoxycytidine triphosphatase (dCTPase).

The bacterial host cell in the above described system may further contain a DNA repair template comprising a donor DNA sequence flanked by a left homologous arm and a right homologous arm. The donor DNA sequence comprises at least one desired mutation to the bacteriophage genome. The at least one desired mutation may be a single point mutation or multiple point mutations. The at least one desired mutation may also be insertions and deletions to a DNA sequence of a bacteriophage phage genome. The left and right homologous arms are DNA segments or fragments immediately upstream or downstream of a target DNA sequence. The left and right homologous arms may flank a desired mutation or edit including a mutation site or multiple mutation sites, an insertion of a modified DNA sequence or a foreign DNA sequence, or a deletion of at least a portion of a bacteriophage gene that has the target DNA sequence. The left and right homologous arms are sufficient long to allow the donor DNA sequence being introduced into the bacteriophage genome by homologous recombination. In some embodiments, as short as 50-bp homologous flanking arms are sufficient to generate recombinants that may be selected under the pressure of CRISPR-Cas9 nuclease. In some embodiments, the left homologous arm and the right homologous arm have a length of about 50 bp to about 1.2 kb.

The above described a DNA repair template further include a sequence to introduce one or more mutations to a PAM immediately following a target protospacers, thus the donor DNA sequence would not be a target for Cas9 cleavage. In some embodiments, a DNA sequence of the PAM maybe altered with one or more silent mutations that do not change the amino acid sequence encoded by a target gene but block Cas9 cleavage of the DNA repair template.

Further, in the above described system, the DNA repair template is included in a donor vector such as a plasmid or one of other proper vectors. A bacterial host cell therefore may include two plasmids, one plasmid for producing Cas9 protein and a guide RNA targeting a target DNA sequence in the bacteriophage genome, and the other plasmid carrying a donor DNA providing one or more mutations or edits to the target DNA sequence.

In the above described system, the bacterial host cell may further be infected by a bacteriophage and then contain a genome of the bacteriophage.

Alternatively, an engineered system for editing a bacteriophage genome may comprise a bacterial host cell that is adapted to produce an engineered bacteriophage through CRISPR-Cas. The bacterial host cell comprises a first nucleic acid sequence encoding a Cas protein, and at least one nucleic acid sequence encoding at least one guide RNA (gRNA). In some preferred embodiments, the Cas protein is type II CRISPR-associated nuclease enzyme Cas9 derived from Streptococcus pyogenes. However, any applicable type or modified CRISPR enzyme may be used. The at least one guide RNA may include components of a trans-activating crRNA (tracrRNA) and two or more guide sequences. The at least one guide RNA may be a single guide RNA (sgRNA) including the tracrRNA connected to the two or more guide sequence. The two or more guide sequences are respectively complementary to two or more target DNA sequences in a bacteriophage genome. The first nucleic acid sequence and the at least one nucleic acid sequence encoding the at least one guide RNA are operably linked to a same or different regulatory elements operable in the bacterial host cell, on same or different vectors, such that the Cas9 protein and the at least one gRNA are expressed and form at least one CRISPR-Cas complex in the bacterial host cell, wherein the Cas protein and the at least one gRNA do not naturally occur together, i.e. they are engineered to occur together.

In some embodiment, the above described first nucleic acid sequence encoding the Cas protein and the at least one nucleic acid sequence encoding at least one guide RNA are located in a same CRISPR-Cas spacer vector, such as a same plasmid, and are operably linked to a same regulatory element operable in the bacterial host cell. The Cas protein and the at least one guide RNA are constitutively expressed. Each of the one or more target DNA sequences is a protospacer immediately preceding a protospacer adjacent motif (PAM) in a gene of the bacteriophage.

In one embodiment, the bacteriophage in the above described system is bacteriophage T4. In another embodiment, the above described bacteriophage is a glucosylhydroxymethyl cytosine (ghmC)-unmodified mutant phage, which makes the bacteriophage more vulnerable to cleavage by Cas9 endonucleases. A nucleic acid sequence encoding the two or more guide sequences comprises a spacer of about 20 nucleotides (nt) from a gene encoding bacteriophage capsid protein. The nucleotide sequence for producing a guide sequence (crRNA) may be a sequence of any one of SEQ ID NOS 1-25.

For example, a nucleic acid sequence encoding the guide sequence may comprise a spacer of about 20 nucleotides (nt) from a gene encoding bacteriophage capsid protein. The nucleic acid sequence encoding the guide sequence may be a sequence set forth in SEQ ID NO: 1 or 19. In some other embodiment, a nucleic acid sequence encoding the guide sequence comprises a spacer of about 20 nucleotides (nt) from a gene encoding a bacteriophage portal protein. The nucleic acid sequence encoding the guide sequence may comprise a sequence set forth in SEQ ID NO: 2 or 13.

In the above described engineered system, the at least one guide RNA may comprise two guide sequences including a first guide sequence being complementary to a first target DNA sequence and a second guide sequence being complementary to a second target DNA sequence. The first target DNA sequence and the second target DNA sequence are two adjacent protospacers immediately preceding two respective PAM sequences in a bacteriophage gene. Further, in some embodiments, the bacterial host cell may be infected by a bacteriophage. As a result, a genome of the bacteriophage is contained in the bacterial host cell. The genome of the bacteriophage includes the two adjacent protospacers immediately preceding the two respective PAM sequences.

The above described bacterial host cell in the above described engineered system may further contain a DNA repair template comprising a donor DNA sequence flanked by a left homologous arm and a right homologous arm, the donor DNA sequence comprising a mutation to the bacteriophage genome, the left and right homologous arms being sufficient long to allow the donor DNA sequence being inserted into the bacteriophage genome by homologous recombination. In some embodiment, a donor plasmid comprising the DNA repair template is carried by the bacterial host cell. Further, the bacterial host cell contains a genomic DNA of the bacteriophage. The genomic DNA includesthe one or more target DNA sequences.

Further, in the above described system, the bacteriophage that may be bacteriophage T4. In some embodiments, the above bacteriophage is a wild-type bacteriophage. In some embodiments, the above bacteriophage is a glucosylhydroxymethyl cytosine (ghmC)-unmodified mutant phage. The ghmC-unmodified mutant phage may contain an amber mutation in gene 42 that codes for deoxycytidine monophosphate hydroxymethylase (g42) and an amber mutation in gene 56 that codes for deoxycytidine triphosphatase (dCTPase).

In some embodiments, the bacterial host cell in the above described system may further contain a DNA repair template comprising a donor DNA sequence flanked by a left homologous arm and a right homologous arm. The donor DNA sequence further comprises at least one desired mutation to the bacteriophage genome. The at least one desired mutation may be a single point mutation or multiple point mutations. The at least one desired mutation may also be insertions and deletions to a DNA sequence of a bacteriophage phage genome. The left and right homologous arms are DNA segments or fragments immediately upstream or downstream of a target DNA sequence. The left and right homologous arms may flank a desired edit including a mutation site or multiple mutation sites, an insertion of a modified DNA sequence or a foreign DNA sequence, or a deletion of at least a portion of a bacteriophage gene that has the target DNA sequence. The left and right homologous arms are sufficient long to allow the donor DNA sequence being introduced into the bacteriophage genome by homologous recombination. In some embodiments, as short as 50-bp homologous flanking arms are sufficient to generate recombinants that may be selected under the pressure of CRISPR-Cas9 nuclease. In some embodiments, the left homologous arm and the right homologous arm have a length of about 50 bp to about 1.2 kb.

In another aspect, embodiments provide one or more vectors that may be delivered into an above described bacterial host cell for producing components in the above described systems. Particularly, in some embodiments, the one or more vectors include a CRISPR-Cas spacer vector that comprises a first nucleic acid sequence encoding a Cas protein, and a second nucleic acid sequence encoding a guide RNA (gRNA) comprising a trans-activating crRNA (tracrRNA) and a guide sequence complementary to a target DNA sequence in a bacteriophage genome. The guide sequence is able to be hybridized to the target DNA sequence in the bacteriophage genome. The first nucleic acid sequence and the second nucleic acid sequence are operably linked to a same regulatory element or different regulatory elements operable in a bacterial host cell containing the CRISPR-Cas vector, thereby the Cas protein and the gRNA being able to be expressed in the bacterial host cell and form a CRISPR-Cas complex therein. Preferably, the first nucleic acid sequence and the second nucleic acid sequence are operably linked to a same regulatory element such as a same promoter. Preferably, the regulatory element provides constitutive expression for Cas protein and the gRNA in a bacterial host cell. In some embodiments, the guide sequence is upstream of the tracrRNA. When expressed, the guide sequence directs sequence-specific binding of a CRISPR-Cas complex to a target sequence in a bacteriophage genome. It should be appreciated that the Cas protein and the gRNA do not naturally occur together.

Alternatively, a trans-activating crRNA (tracrRNA) and a guide sequence complementary to a target DNA sequence in a bacteriophage genome are located in different vectors in the one or more vectors. Accordingly, embodiments provide a first vector including an expression cassette for producing Cas protein and a second vector that may be used to specifically produce a guide RNA. The expression cassette in the first vector may include a first regulatory element operably linked to a first nucleic acid sequence encoding the Cas protein. The Cas protein may be a Cas9 protein derived from Streptococcus pyogenes. The first regulatory element includes a promoter to regulate the expression of the Cas protein. In some embodiments, the first regulatory element allows the Cas protein to be consistently expressed in a bacterial host cell. Embodiments further provide a second vector that may have an expression cassette comprising a second nucleic acid sequence encoding at least one guide RNA (gRNA) operably linked to a second regulatory element that is operable in a bacterial host cell. Preferably, the second regulatory element is a promoter and allows the at least one guide RNA to be consistently expressed in a bacterial host cell. The at least one guide RNA (gRNA) may comprise a trans-activating crRNA (tracrRNA) and one or more guide sequences respectively complementary to one or more target DNA sequences in a bacteriophage genome.

In some embodiments, the one or more guide sequences are upstream of the tracrRNA. When expressed, the one or more guide sequences direct sequence-specific binding of at least one CRISPR-Cas complex to one or more target sequences in a bacteriophage genome. Each of the one or more target sequences in a bacteriophage, as described, is a protospacer immediately preceding a protospacer adjacent motif (PAM). The protospacer may be in a gene for a protein product.

The above described vectors, wherein a nucleic acid sequence codes for a guide sequence comprises a sequence of a spacer corresponding to a target protospacer in a bacteriophage genome. The sequence of the spacer may be one of SEQ ID NOS 1-25. In some embodiments, a spacer may be a sequence of about 20 nucleotides (nt) derived from a gene encoding bacteriophage capsid protein or a bacteriophage portal protein. In some embodiments, a spacer comprises a sequence of about 20 nucleotides (nt) from a gene encoding a bacteriophage portal protein. In some embodiment, a spacer may have a sequence set forth in SEQ ID NO: 1 or 19. In some embodiment, a spacer may have a sequence set forth in SEQ ID NO: 2 or 13.

Embodiments further provide a vector that comprises a nucleic acid sequence encoding at least one guide RNA comprising two guide sequences. The two guide sequences are respectively complementary to two target DNA sequences in a target gene of a bacteriophage. The two target DNA sequences are two adjacent protospacers immediately preceding two respective PAM sequences in a bacteriophage gene.

Further, embodiments provide a vector comprising a DNA repair template. The DNA repair template includes a donor DNA sequence flanked by a left homologous arm and a right homologous arm. The donor DNA sequence comprising one or more desired mutations to be introduced into the bacteriophage genome to alter the bacteriophage genome or a gene product of the bacteriophage. The one or more desired mutations may be a single point mutation, multiple point mutations, insertion, or deletion. The left homologous arm and the right homologous arm are DNA segments homologous to end sequences of a double-strand break created by a Cas protein in a target DNA sequence in the bacteriophage genome. The left and right homologous arms are sufficient long to allow the donor DNA sequence to be inserted into the bacteriophage genome by homologous recombination. In some embodiment, the left and right homologous arms have a length of about 50 bp to about 1.2 kb. In some embodiment, a donor plasmid comprising the DNA repair template is provided.

In another aspect, embodiments of the present invention provide a kit for engineering a bacteriophage genome or introducing alteration, such as a single point mutation, multiple point mutations, insertion, or deletion, into genomic DNA of a bacteriophage. The kit comprises components in the above described systems. The components include the above described one or more vectors. These components may be separately prepared, packaged, and/or stored. In some embodiments, in addition to the one or more vectors, the kit further comprises above described bacterial host cells. A host cell may be a wild type bacterial cell or a bacterial cell transfected with one or more above described vectors. In some embodiments, the kit further comprises a glucosylhydroxymethyl cytosine (ghmC)-unmodified mutant bacteriophage, which is not resistant to most restriction endonucleases.

In another aspect, embodiments of the present invention provide a method for cutting a bacteriophage genome at a specific site using the above disclosed vectors and system. The method comprises introducing a bacteriophage into a bacterial host cell containing components such as a Cas protein and a guide RNA (gRNA). The Cas protein and the guide RNA form a CRISPR-Cas complex in the bacterial host cell. It should be appreciated that the Cas protein and the guide RNA do not naturally occur together, i.e. they are engineered to occur together. In some embodiments, the Cas protein may be a type II CRISPR-associated nuclease enzyme Cas9 derived from Streptococcus pyogenes. However, a nuclease used to cleave the bacteriophage genomic DNA is not limited to Cas9. Other types of Cas nuclease may be suitable to use. In some embodiments, the guide RNA is a single guide RNA comprising tracrRNA connected to a guide sequence. In some embodiments, the tracrRNA is not connected with a single guide RNA but may be hybridized to the guide RNA. A guide RNA may also include tracrRNA and one or more guide sequences. Each guide sequence is complementary to a target DNA sequence in a bacteriophage genome and may hybrids to the target DNA sequence. Guided by the guide RNA, the CRISPR-Cas complex binds to the target DNA sequence and effectively cleaves the target DNA sequence, creating a double-strand break in the bacteriophage genome.

Embodiments further provide a method of editing a bacteriophage genome. The method comprises introducing a bacteriophage into a bacterial host cell containing a CRISPR-Cas spacer vector and a DNA repair template. The CRISPR-Cas spacer vector comprises a first nucleic acid sequence encoding a Cas9 protein, and at least one nucleic acid sequence encoding at least one guide RNA (gRNA) comprising a trans-activating crRNA (tracrRNA) and one or more guide sequences respectively complementary to one or more target DNA sequences in a bacteriophage genome. The first nucleic acid sequence and the at least one nucleic acid sequence encoding the at least one guide RNA are operably linked to a regulatory element operable in the bacterial host cell. The Cas9 protein and the at least one gRNA are expressed and form at least one CRISPR-Cas complex in the bacterial host cell, wherein the Cas protein and the at least one gRNA do not naturally occur together. The at least one gRNA targets the one or more target DNA sequences in the bacteriophage genome and guide the Cas9 protein to cleave the bacteriophage genome at the one or more target DNA sequences, thereby generating one or more double-strand breaks therein. The DNA repair template includes a donor DNA sequence flanked by DNA segments homologous to end sequences of one of the one or more double-strand breaks, and the donor DNA sequence includes at least one desired mutation. The donor DNA sequence being inserted into one of the one or more double-strand breaks through homology directed repair, thereby altering the expression of a bacteriophage gene. The DNA repair template may be included in a Cas9-resistant donor plasmid as a portion thereof.

Preferably, the donor DNA sequence includes a mutation to a PAM immediately following a target protospacers, thus the donor DNA sequence would not be a target for Cas9 cleavage. In some embodiments, a sequence of the PAM maybe altered with one or more silent mutations that do not change the amino acid sequence encoded by a target gene but block Cas9 cleavage of the DNA repair template.

In some embodiments, the CRISPR-Cas spacer vector is a plasmid, and the Cas9 protein and the at least one guide RNA are constitutively expressed in the bacterial host cell. Each of the one or more target DNA sequences may be a protospacer immediately preceding a protospacer adjacent motif (PAM) in a gene of the bacteriophage.

In some embodiments, the bacteriophage used for editing of genomic DNA is bacteriophage T4. The bacteriophage may be a wild type. Alternatively, the bacteriophage may be a glucosylhydroxymethyl cytosine (ghmC)-unmodified mutant phage, which makes the bacteriophage more vulnerable to cleavage by Cas9 endonucleases.

Alternative, a bacteriophage genome is introduced into a bacterial host cell contains a CRISPR-Cas spacer vector and a DNA repair template. The CRISPR-Cas spacer vector comprises an expression cassette to express both the Cas protein and the guide RNA. The expression cassette comprises a regulatory element operable in the bacterial host cell, a first nucleic acid sequence encoding a Cas protein, and a second nucleic acid sequence encoding a guide RNA (gRNA) comprising a trans-activating crRNA (tracrRNA) and a guide sequence (crRNA). The first nucleic acid sequence and the second nucleic acid sequence are operably linked to the regulatory element. Under the guide of the gRNA with the guide sequence, Cas protein cleaves the bacteriophage genome at one site in the target DNA sequences hybridized to the guide sequence and form one double-strand break in the target DNA sequence.

Alternatively, a guide RNA comprises a tracrRNA connected to two guide sequences. The two guide sequences include a first guide sequence being complementary to a first target DNA sequence and a second guide sequence being complementary to a second target DNA sequence. The first target DNA sequence and the second target DNA sequence are two adjacent protospacers immediately preceding two respective PAM sequences in a bacteriophage gene. Under the guide of these two guide sequences, the Cas9 protein cleaves the bacteriophage gene at two adjacent sites in the two adjacent protospacers of a bacteriophage genome, thereby creating a double-strand break in the bacteriophage genome with an intervening sequence between the two adjacent sites being excised. The DNA repair template includes a donor DNA sequence flanked by DNA segments homologous to end sequences of the double-strand break, allowing the excised intervening sequence being replaced by the donor DNA sequence through a homologous recombination to repair the double-strand break. The DNA segments homologous to end sequences of one of the one or more double-strand breaks, which are left and right homologous arms, are sufficient long to allow the donor DNA sequence being introduced into the bacteriophage genome by homologous recombination. In some embodiments, the left and right homologous arms have a length of about 50 bp to about 1.2 kb.

In some embodiments, the above described method further includes co-delivering into the bacterial host cell a CRISPR-Cas spacer vector and the DNA repair template. The DNA repair template may be delivered into the bacterial host cell as a portion of a donor vector, such as a donor plasmid.

In some embodiments, the above described method further includes selecting a proper spacer to construct CRSPR-Cas spacer vector that allows the cleavage of a Cas protein to construct a CRISPR-Cas spacer vector. In some embodiments, the proper spacer is a spacer of about 20 nucleotides (nt) from a genomic DNA sequence of the bacteriophage for encoding the one or more guide sequences. In some embodiments, the spacer may have a nucleic acid sequence set forth in any one of SEQ ID NOS 1-25, as listed in Table 1. A spacer may be of about 20 nucleotides (nt) from a gene encoding bacteriophage capsid protein is used as a nucleic acid sequence encoding a guide sequence. For example, in some embodiments, a spacer comprises a sequence set forth in SEQ ID NO: 1 or 19. In some embodiments, a spacer may comprise a sequence of about 20 nucleotides (nt) from a gene encoding a bacteriophage portal protein. For example, in some embodiments, a spacer comprises a sequence set forth in SEQ ID NO: 2 or 13.

In some embodiments, the above described one or more CRISPR-CAS spacer vectors are one or more plasmids having one or more promoters that regulate at least one guide RNA to be constitutively expressed in the bacterial host cell. In some embodiments, the bacteriophage introduced into the bacterial host cell is bacteriophage T4. The bacteriophage may also be glucosylhydroxymethyl cytosine (ghmC)-unmodified mutant phage, which makes the bacteriophage more vulnerable to cleavage by Cas9 endonucleases.

In some embodiments, the above described method further comprises performing plaque assays to bacterial host cell introduced to a wild-type bacteriophage or a ghmC-unmodified mutant of bacteriophage and picking up single plaques to screen bacteriophages that have the desired mutations in their genome, thereby obtaining desired mutant bacteriophage. The screening may be done by sequencing. The mutant bacteriophage may produce desired mutant protein or may be CRISPR-escape mutant (CEM) phage that survive from a cleavage of Cas9.

In another aspect, the present invention provides a method of determining an essentiality of a target gene of a bacteriophage. The method including introducing a null mutation to a target gene of a bacteriophage genome using the engineered system described herein. The null mutation is provided by a DNA repair template, causing the target gene to fail to be translated into a function protein product. The method further includes performing a plaque assay for bacterial host cells infected with the bacteriophage having the null mutation and for bacterial host cells infected with wild type bacteriophage. The target gene is determined to be nonessential if plaque formation for infection of bacterial host cells with bacteriophage that has the null mutation is similar to plaque formation for infection of bacterial host cells with wild type bacteriophage. In some embodiments, the null mutation is an amber mutation. In some embodiments, the null mutation includes a deletion of at least a portion of the target gene, the null mutation being introduced into the genome of the bacteriophage by the method of claim 56.

In another aspect, the present invention provides a method of selecting CRISPR-escape mutation (CEM) bacteriophages under a pressure of CRISPR-Cas9 nuclease. CRISPR-escape refers to that phages survive from cleavage of Cas9 protein in a CRISPR-containing bacteria. In some embodiments, the method of selecting CEM phages comprises infecting bacterial host cells, such as E. coli DH5a, containing one or two of the above described CRISPR-Cas spacer vectors with wild type bacteriophage, picking up first generation (G1) CEM bacteriophage from a first generation plaque, repeating the infecting and picking up steps, until phages are picked up and collected from a third generation (G3) of plaques, thereby obtaining a third generation (G3) of CEM bacteriophage. Further using The G3 CEM bacteriophage to infect the bacterial host cell to obtain single plaques from a progeny produced. In some embodiment, picking up plaque is done after 315 minutes of infection and the collected phage is sequenced.

In some embodiment, a CRISPR-Cas spacer vector used for selecting CEM phages comprises a second nucleic acid sequence encoding the one or more guide sequences comprises a spacer of about 20 nucleotides (nt) from a gene encoding bacteriophage capsid protein. For example, the nucleic acid sequence encoding the one or more guide sequences targeting a target DNA sequence comprises a sequence set forth in SEQ ID NO: 1 or 19. In another embodiment, a CRISPR-Cas spacer vector used for selecting CEM phages comprises a second nucleic acid sequence encoding the one or more guide sequences comprises a spacer of about 20 nucleotides (nt) from a gene encoding bacteriophage portal protein. For example, the nucleic acid sequence encoding the one or more guide sequences targeting a target DNA sequence comprises a sequence set forth in SEQ ID NO: 2 or 13.

Having described the many embodiments of the present invention in detail, it will be apparent that modifications and variations are possible without departing from the scope of the invention defined in the appended claims. Furthermore, The description of the embodiments of the present invention is enhanced by various following examples. It should be appreciated that the following examples are given for the purpose of illustrating various embodiments of the present invention and are not meant to limit the present invention in any fashion. The present examples, along with the methods described herein are presently representative of embodiments, are exemplary, and are not intended as limitations on the scope of the invention.

### EXAMPLES

### Example 1

### Materials and Methods

### Bacterial and Bacteriophage Strains

E. coli strains DH5α (hsdR17(rK-mK+) sup²), CR63 (sup¹λ^{r}), B834 (hsdR_{B} hsdM_{B}met thi sup⁰), P301 (sup⁰), and B40 (sup¹) were used in the experiments described below. WT T4 phage was propagated on E. coli P301(sup⁰) as described previously (46). T4(C) is a mutant containing an amber mutation at amino acid 58 of gene 42 that codes for dCMP hydroxymethylase and an amber mutation at amino acid 124 of gene 56 (30) that codes for dCTPase (19). To prevent accumulation of spontaneous revertants, the T4(C) mutant was propagated on E. coli B834 (hsdR_{B} hsdM_{B} met thi sup⁰) for only one generation. The T4(C) phage stocks containing revertant phage at a frequency of <10⁻⁶ were used for all the experiments. For some experiments, the T4(C) phage was propagated on suppressor-plus E. coli strain CR63 to produce phage with modified cytosines in the genome.

### Plasmids

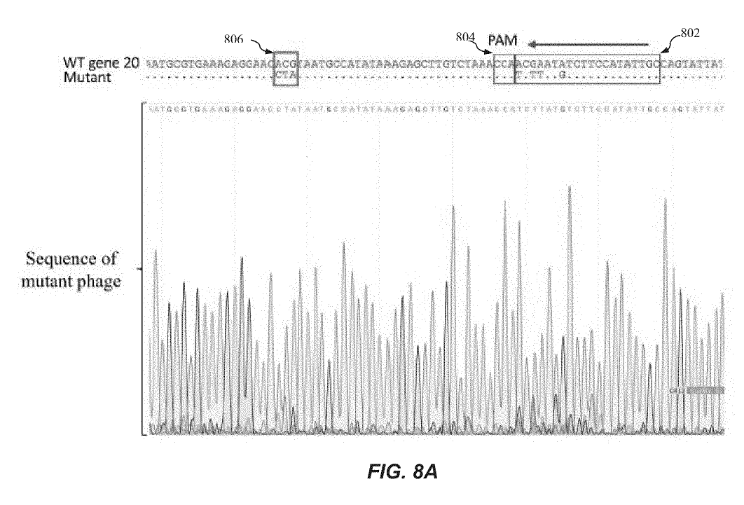

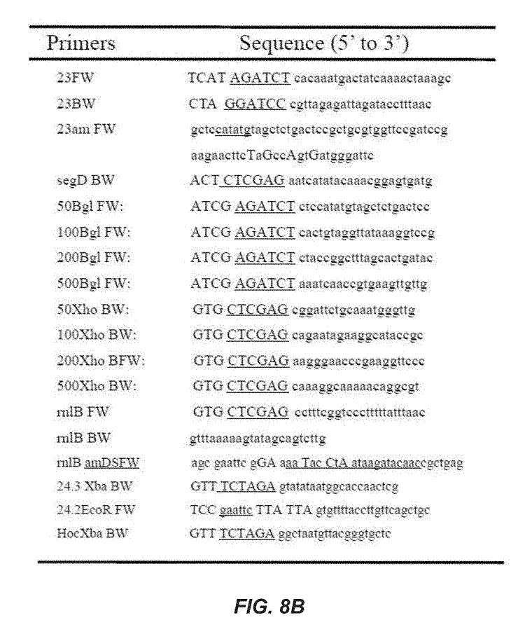

CRISPR-Cas9 spacer plasmids were constructed by cloning spacer sequences into the streptomycin-resistant plasmid DS-SPCas (Addgene no. 48645) (29). Sequences of the spacers are shown in Table 1. The homologous donor plasmids were constructed by cloning the donor DNA into the pET28b vector. FIG. 8B is a table illustrate exemplary primers used for donor DNA constructions. The donor plasmid containing an amber mutation in g23 at amino acid 468 was constructed by two rounds of cloning. First, the full length g23 was amplified with primers 23FW and 23BW using T4 genomic DNA and cloned into the pET28b DNA linearized with BglII and BamHI restriction enzymes to generate pET-23. A DNA fragment containing partial genes of segD and gp23 with the amber mutation was amplified from T4 genome DNA using primers, 23am FW and segD BW, and cloned into pET-23 DNA linearized with NdeI and XhoI to generate the g23 donor plasmid. The donor plasmid was then used as PCR template to amplify homologous arms of different lengths (50-500 bp). These were inserted into the pET28b DNA linearized with BglII and XhoI. The primer sequences are shown in FIG. 8B. FIG. 8B is a table illustrate exemplary primers used for donor DNA constructions.

A DNA fragment containing a multiple mutations in g56 gene between amino acids 121 and 158 was synthesized by Genscript (Piscataway, N.J.). The DNA was inserted into the pET-28b DNA linearized with BglII and BamHI to generate the g56 donor plasmid.

The rnlB donor plasmids were constructed by two rounds of cloning. First, the full length rnlB was amplified with primers rnlB FW and rnlB BW using T4 genome DNA as template. The DNA was then cloned into pET28b linearized with XhoI and EcoRI to generate pET-rnlB. To introduce an amber mutation, a DNA fragment containing the first 300 bp of rnlB and gene 24.2 was amplified from T4 genome using primers 24.3Xba BW and rnlBamDSFW into which an amber mutation was introduced at amino acid 95. The PCR product was then cloned into the pET-rnlB DNA linearized with EcoRI and XbaI to generate pET-rnlB amber. To construct rnlB deletion donor plasmid, a DNA fragment between nt639 of Hoc and nt145 of gene 24.2 was amplified using the T4 genome as a template and primers 24.2EcoR FW and HocXba BW. The DNA was then cloned into pET-rnlB DNA linearized with EcoRI and XbaI to generate pET-rnlB deletion. The primers are shown in FIG. 8B, which is a table illustrating exemplary primers used for donor DNA constructions. The accuracy of all the constructs was confirmed by DNA sequencing.

### Plaque Assays

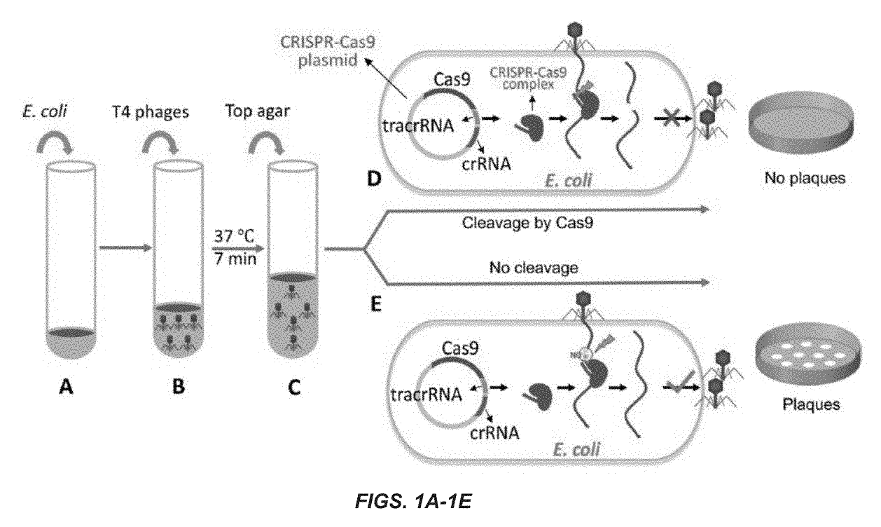

The efficiency of individual spacer plasmids to restrict T4 phage infection was determined by plaque assay as shown in FIGS. 1A-1E. The CRISPR-Cas plasmids with different spacers were transformed into E. coli strains DH5α or B834. Up to ˜10⁶ plaque-forming units (PFU) of either WT T4 or T4 (C) in 100 μl of Pi-Mg buffer (26 mM Na₂HPO₄, 22 mM KH₂PO₄, 70 mM NaCl, and 1 mM MgSO₄) was added to 300 al of E. coli (˜10⁸ cells/ml) containing the CRISPR-Cas plasmid. After incubation for 7 min at 37° C., 3 ml of 0.7% top agar with streptomycin (50 μg/ml) was added into each tube, mixed, and poured onto LB-streptomycin plates. Serial dilutions were made where necessary. The plates were incubated at 37° C. overnight. The efficiency of plating (EOP) was calculated by dividing the PFU produced from infection of E. coli containing a spacer by the input PFU.

### DNA Sequencing of Single Plaques

Single plaques were picked using a sterile Pasteur glass pipette and transferred into a 1.5 ml Eppedoff tube containing 200 al Pi-Mg buffer (26 mM Na₂HPO₄, 68 mM NaCl, 22 mM KH₂PO₄, 1 mM MgSO₄, pH 7.5) plus 2 μl chloroform. After 1 h incubation at room temperature with mixing every few min, 4 μl of the sample was used as a template for PCR using Phusion High-Fidelity PCR Master Mix (Thermo Fisher Scientific). Prior to starting PCR, the phage was denatured at 95° C. for 10 min. Amplification was performed using appropriate primers flanking the protospacer sequence. The amplified DNA was purified by agarose gel electrophoresis using QIAquick Gel Extraction Kit (Qiagen) and was sequenced (Retrogene).

### Genome Editing

The donor plasmid and the corresponding CRISPR-Cas plasmid were co-transformed into suppressor containing E. coli strain such as B40 (sup¹). B40 cells transformed with either the donor plasmid or the CRISPR plasmid were used as controls. The cells were infected with WT or T4(C) mutant as described above. The progeny plaques produced were analyzed for genetic markers or the genome was amplified and sequenced as described above.

### One-Step Growth Experiment

Log phase E. coli cells (˜2×10⁸ cells per ml) grown on 1:1 LB/M9CA medium were infected with phage at a multiplicity of infection (m.o.i.) of 1 at 37° C. Five minutes after infection, 100 μl of the 10⁵-times diluted mixture was plated to determine the number of infected cells. Samples were withdrawn from the same mixture every 5 min until 60 min. The cells were lysed by adding a few drops of chloroform and DNAse 1 (7 μg/ml). The phage titer was determined by plaque assay following serial dilutions. Burst size is defined as the number of progeny phage produced per infection cell.

### Statistics

Each experiment was repeated at least three times. The data were initially analyzed by a two-way analysis of variance (ANOVA), followed by a Bonferonni post hoc test to compare individual groups using Prism Graphpad software.

### Selection of CRISPR-Cas Escape Mutants (CEMs)

Three hundred μl of E. coli DH5α containing the CRISPR-Cas plasmid (˜10⁸ cells/ml) were infected with WT T4 and mixed with 3 ml of 0.7% top agar with streptomycin (50 μg/ml), and poured onto a LB-streptomycin plate. After overnight incubation at 37° C., the plaques formed (Generation 1, G1) were picked by stabbing on each plaque with a sterile toothpick and transferring to another LB-streptomycin plate. The plaques formed (G2) are then subjected to the same process two to three more times (G3 to G5). Single plaques at each stage were sequenced as described above after amplification of the regions flanking the protospacer sequence using appropriate primers. For the 20-995 spacer, 1172 bp upstream and 226 bp downstream flanking regions were amplified; for 20-1070, 1247 bp upstream and 151 bp downstream flanking regions were amplified; for 23-1490, 227 bp upstream and 798 bp downstream flanking regions were amplified; and for 23-2, 129 bp upstream and 896 bp downstream flanking regions were amplified. E. coli DH5α without the CRISPR-Cas plasmid was used as a control.

### Evolution of CEMs

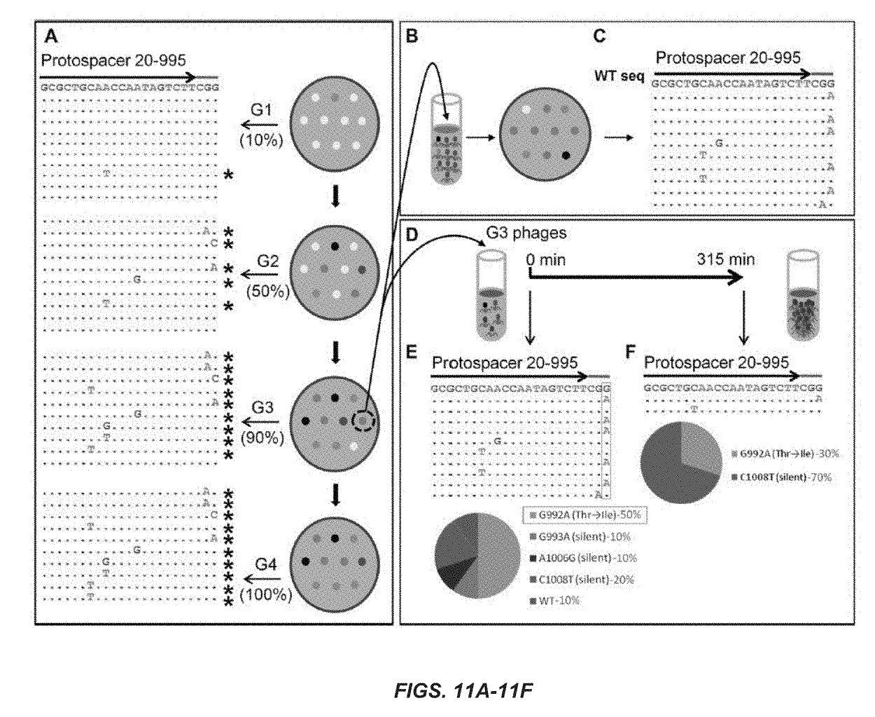

Evolution of phages isolated from a single plaque was carried out as shown in FIG. 11D. Single plaque was picked and transferred into a 1.5 ml Eppedoff tube containing 1 ml Pi-Mg buffer plus 10 μl chloroform. The phage titer was determined by plaque assay following serial dilutions. Four milliliters of log phase E. coli DH5α cells (˜2×10⁸ cells per ml) containing the CRISPR-Cas plasmid were infected with phages at an m.o.i. of 0.001 at 37° C. Three hundred and fifteen minutes after infection, 400 al of culture was collected and treated with a few drops of chloroform and deoxyribonuclease I (7 μg/ml) and lysozyme (10 μg/ml) were then added to the sample and incubated at 37° C. for 1 h. The cell debris was removed by centrifugation of the suspension at 7,000 rpm (4,300×g) for 10 min at 4° C. The supernatant was transferred into a new tube and the phages were pelleted by centrifugation for 45 min at 15,000 rpm (21,130 g) at 4° C. The pellet was resuspended in 200 al of Pi-Mg buffer, serially diluted and plated on LB plates. Ten single plaques were picked and sequenced as described above.

### Culture of Spacer 20-995 CEMs

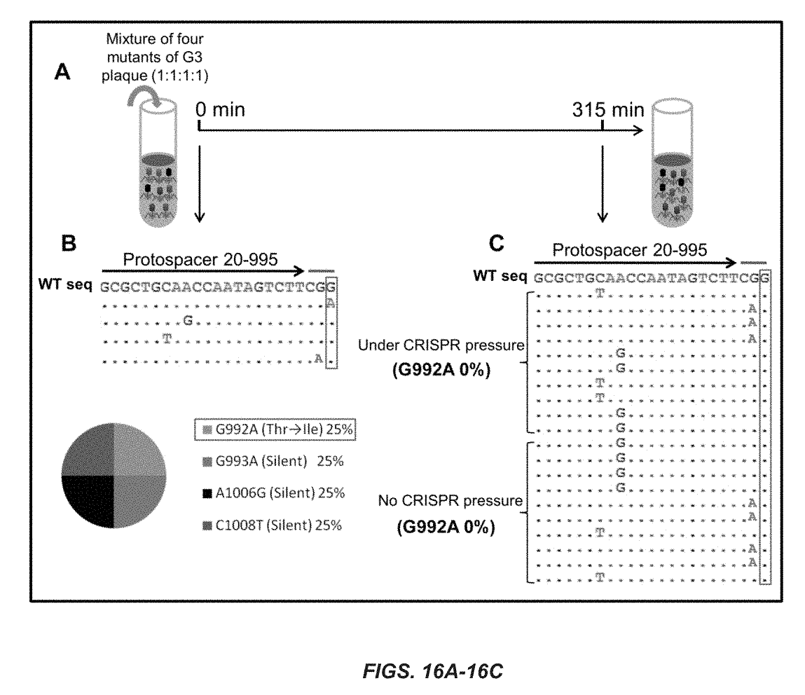

An equal number of PFU of four T4 CEMs were mixed (FIGS. 16A, 16B, and 16C) and added to 1 ml of the log phase E. coli DH5α cells (˜2×10⁸ cells per ml) at an m.o.i. of 0.001 at 37° C. Three hundred and fifteen min after infection, 100 μl of culture was collected and treated as described above. Ten single plaques were picked and sequenced.

### Plate Spot Test

Temperature sensitivity of each phage mutant was determined by plate spot test as described previously(19). Briefly, 300 al of E. coli DH5α (˜10⁸ cells/ml) was mixed with 3 ml of 0.7% top agar, and poured onto a LB plate. About 1-μl of phage suspension (10⁰ to 10⁴ PFU) was applied on the top agar plate and left for 3-5 min at room temperature to let the drops dry. Three identical plates were prepared and incubated overnight at 42° C., 37° C., and 25° C. respectively.

### Example 2

Cytosine Modification of Phage T4 Genome Inhibits, but does not Block, Restriction by CRISPR-Cas9 Nuclease

FIGS. 1A-1E illustrate an experimental scheme for testing the effect of CRISPR-Cas on phage T4 infection according to one embodiment of the present invention. E. coli cells containing a CRISPR-Cas plasmid (˜3×10⁶ cells) shown in FIG. 1A are mixed with phage T4 (up to 10⁶ PFU), as shown in FIG. 1B. FIG. 1C shows that after incubation at 37° C. for 7 minutes, top agar is added and the mixture is poured onto LB plates. The plates are incubated overnight at 37° C. FIG. 1D shows that cleavage by CRISPR-Cas9 nuclease at the protospacer sequence disrupts the phage genome resulting in loss of plaque-forming ability. FIG. 1E shows that if the genome is resistant to Cas9 cleavage, plaques form at frequency similar to that of the control vector plasmid. See Materials and Methods for more details.

To determine if the ghmC-modified WT T4 genome may be inactivated by CRISPR-Cas9 nuclease, 25 recombinant plasmids each containing a different 20-nt spacer sequence (Table 1) are constructed and transformed each into E. coli, as shown in FIG. 1A. As shown in Table 1, the spacer sequences are distributed all over the T4 genome and contained different percentages of cytosine nucleotide. To exclude any potential interference of DNA adenine methylation on CRISPR-Cas9 activity, none of the spacers included the adenine methylation site 5′-GATC-3′.

In the example, these spacers are inserted into the plasmid, DS-SPCas (Addgene plasmid no. 48645) (29), and kept under the control of J23100 promoter which constitutively expresses the corresponding crRNA (see Materials and Methods for details). The crRNA forms a complex with Cas9 and tracrRNA that are also expressed constitutively from the same plasmid. If the crRNA: tracrRNA: Cas9 complex is functional, it cleaves at the protospacer sequence of the T4 genome delivered by phage infection (FIGS. 1B-D). Consequently, the genome is disrupted, which likely leads to a loss of plaque forming ability, with certainty if the disruption occurred in an essential gene (FIG. 1D). However, if the genome is resistant to CRISPR-Cas9 cleavage, plaques appears at nearly the same frequency as the control plasmid lacking the spacer sequence (FIG. 1E).

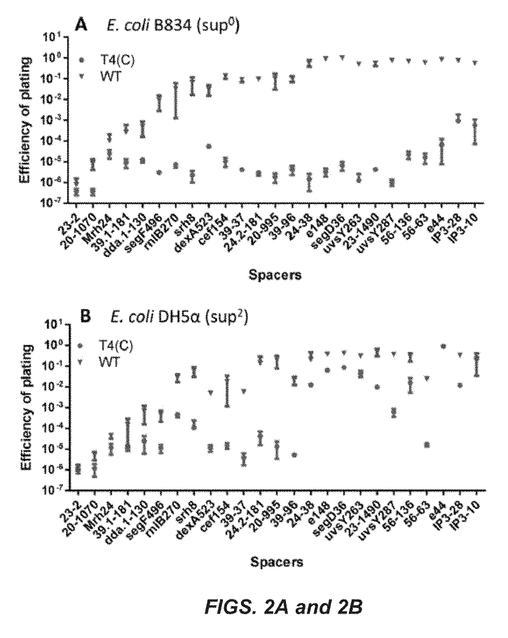

FIGS. 2A and 2B are two graphs showing restriction of phage T4 infection by CRISPR-Cas. In this example, the spacer-containing E. coli cells were infected with WT T4 or T4(C) mutant as per the basic scheme shown in FIGS. 1A-1E. See Materials and Methods for more details. The restriction of phage T4 infection was determined by the efficiency of plating (EOP), as determined by plaque assay. EOP was calculated by dividing the number of PFU produced on the spacer containing E. coli by the number of input PFU. EOP of modified (WT, triangle symbols) or unmodified mutant (T4(C), dot symbols) phages was determined on E. coli strains B834 (sup⁰) (shown in FIG. 2A) or DH5α (sup²) (shown in FIG. 2B). The labels on the X-axis denote the spacer. The sequences of the spacers are listed in Table 1. The experiments were done triplicate.

As shown in FIGS. 2A and 2B, the plating efficiencies of the WT T4 phage and the T4(C) mutant (similar to T4gt mutant (30)) were determined for each of the twenty-five spacer plasmids by plaque assay. The T4(C) mutant has amber mutations in the cytosine hydroxymethylase (g42) and dCTPase (g56) genes. When grown on sup⁰ E. coli, the T4(C) mutant produces phage particles containing unmodified C-genomes. As shown in FIG. 2A, the WT phage containing the modified genome gave higher plating efficiencies when compared to the mutant phage containing the unmodified genome. For most of the spacers, the difference was greater than three logs, many showing four to five log difference. These data, thus, demonstrate that the T4(C) mutant phage was highly restricted by various spacers whereas the restriction of WT phage infection varied greatly depending on the spacer used. For instance, spacers 23-2 and 20-1070, both in the essential genes encoding the major capsid protein gp23 and the portal protein gp20 respectively, highly restricted both ghmC-modified and unmodified phage infections (plating efficiency, ˜10⁻⁶). On the other hand, two other spacers, 23-1490 and 20-995, in the same genes, showed high restriction for the unmodified phage (plating efficiency, ˜10⁻⁵) but poor restriction for the ghmC-modified phage (plating efficiency, ˜10⁻¹). These differences could not be due to differences in the C or GC content of the spacer sequences because no such correlation has been observed (Table 1). Furthermore, when the experiments were repeated in the genetic background of E. coli DH5α (sup²) which suppresses the amber mutations, the restriction of T4(C) mutant was reduced by as much as four logs in most cases (FIG. 2B), in many cases approaching that seen with the WT phage (e.g., spacers segD36, uvsY263).

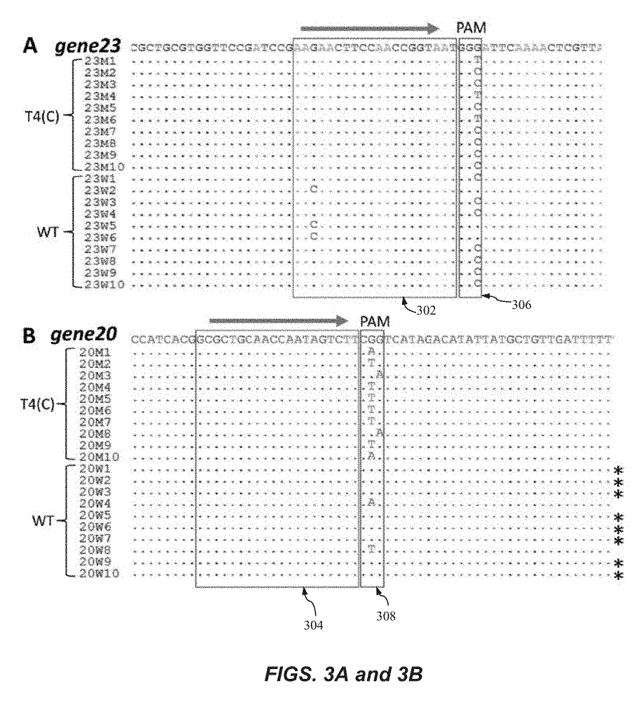

FIGS. 3A and 3B illustrate alignment of CRISPR-Cas escape mutant sequences. The WT and T4(C) mutant phage infections were carried out as per the basic scheme shown in FIGS. 1A-1E. Single plaques that appeared on E. coli containing spacer 23-2 (g23) (A) or 20-995 (g20) (FIG. 3B) were isolated and the DNA flanking the protospacer and PAM sequences was amplified by PCR and sequenced. The arrows show the 5′ to 3′ direction of the spacer sequences. The spacer sequences are marked with red boxes 302 and 304 and the PAM sequences are marked with blue boxes 306 and 308. * indicates the WT sequences.

In the example, preliminary sequencing of the plaques produced from phage infections as above contained mutations in the CRISPR editing region, which presumably allowed the virus to escape the Cas9 nuclease attack. Indeed, the plaques produced from the T4(C) mutant infections all had mutations in the PAM (Protospacer Adjacent Motif) sequence (FIGS. 3A and 3B). In the case of Streptococcus pyogenes Cas9, PAM is a three nucleotide 5′-NGG-3′ sequence immediately downstream of the protospacer sequence that is strictly required for binding of the CRISPR-Cas9 complex. On the other hand, the “CRISPR-escaped” plaques from the WT T4 phage infection showed a different pattern. The plaques generated when the plating efficiency was low (spacer 23-2) contained mutations in PAM or protospacer sequences, similar to that observed with the T4(C) mutant phage infections (FIG. 3A). However, the plaques generated when the plating efficiency was high (spacer 20-995) contained largely WT sequences (8 out of 10) and a few with mutations in PAM sequences (2 out of 10) (FIG. 3B). These results suggest that either the loss of the Cas9 recognition of the PAM sequence or the mismatches in the spacer sequence resulted in the escape of Cas9 cleavage and appearance of plaques. In the case of the WT phage containing ghmC-modified genome, escape could also occur even when there is no mutation in the protospacer or PAM sequences, provided that the protospacer was resistant to Cas9 cleavage. The latter likely involves complex mechanisms that require a detailed investigation, which is in progress.

### Example 3

### Creation of Specific Mutants by Editing of T4 Genome Using CRISPR-Cas9

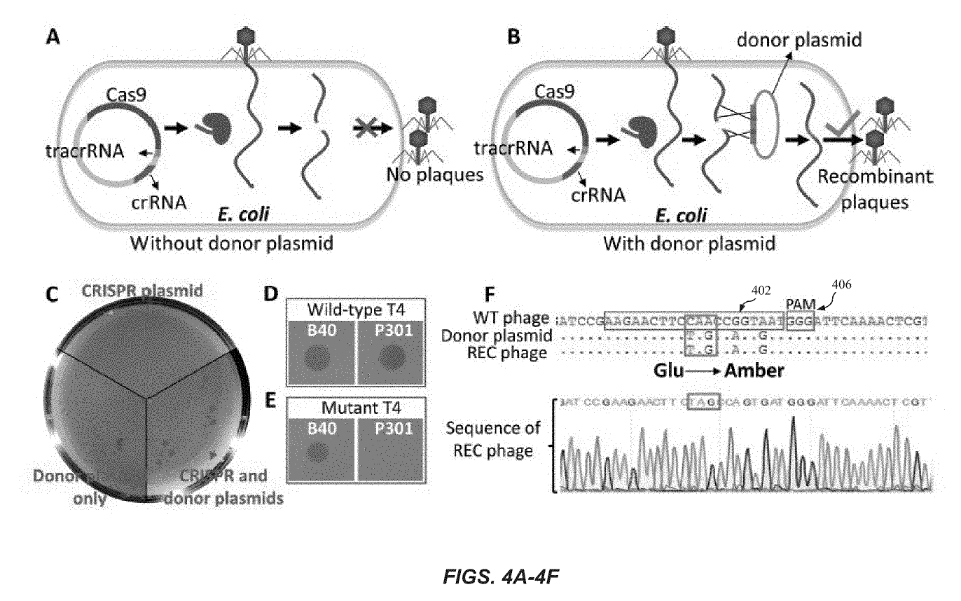

FIGS. 4A-4F illustrate experimental scheme for phage T4 genome editing using CRISPR-Cas9 according to one embodiment of the present invention. T4 phage infections were performed according to the basic scheme shown in FIGS. 1A-1E. FIG. 4A is a schematic illustrating E. coli containing only the spacer plasmid leads to restriction of phage infection. FIG. 4B is a schematic illustrating E. coli containing both the spacer plasmid and a Cas9-resistant donor plasmid allows arms

of the donor plasmid DNA. The resultant recombinant phages are released following lysis. FIG. 4C is an image showing plating of the phage lysates from various infections. FIG. 4D illustrates spot-test of a random plaque generated from E. coli containing only the donor plasmid. FIG. 4E illustrates spot-test of a random plaque generated from E. coli containing both the CRISPR plasmid and the donor plasmid. Spot tests were done on lawns of E. coli B40 (sup¹) and P301 (sup⁰) strains. FIG. 4F shows alignment of sequencing of a random plaque generated from E. coli containing both the CRISPR plasmid and the donor plasmid. It shows the presence of amber mutation and two silent mutations in the recombinant plaque. Protospacer sequence is marked with blue box 402 and PAM sequence is marked with red box 406.

If the modified genome is susceptible to CRISR-Cas9 cleavage, as the above data suggest, it is possible to edit the genome at the cleaved site. To answer a question that if site-directed mutations, for example, an amber mutation may be incorporated at a given position, a second donor plasmid with an amber mutation in the protospacer sequence, along with the CRISPR-Cas spacer plasmid, were co-delivered into the same E. coli (FIGS. 4A-4F). It is predicted that the amber mutation in the protospacer sequence, at codon 468 of the major capsid protein gp23, makes the donor resistant to Cas9 attack (FIGS. 4A and 4B). To make it even more stringent, two additional silent mutations near the amber mutation are introduced to completely block Cas9 cleavage of the donor plasmid (FIG. 4F). No difficulty in transforming or maintaining the donor plasmid under the CRISPR-Cas9 background is encountered. Upon infection of these E. coli cells by WT T4 phage (or T4(C) mutant), the delivered genome is cleaved resulting in inactivation of the genome and loss of plaque forming ability (FIG. 4A). However, since a donor DNA is present, recombination between the cleaved ends and the donor results in the transfer of the amber mutation from donor to T4 genome, which, in addition, restores the integrity of the genome (FIG. 4B). To allow the amber mutant genome survive and produce a plaque, the spacer and donor plasmids were co-transformed into E. coli B40 (sup¹) that constitutively expresses a serine tRNA suppressor which allows translation of full-length and functional gp23. E. coli cells containing either the donor g23 mutant plasmid or the CRISPR-Cas9 plasmid alone were used as controls and the infection was carried out as depicted in FIGS. 1A-lE using the WT T4 phage.

As shown in FIGS. 4A and 4C, no plaques were produced when E. coli containing only the CRISPR-Cas9 plasmid was infected with 500 plaque forming units (PFU) of WT T4 phage. This means that, consistent with the data in FIGS. 2A and 2B, the T4 genome was inactivated by CRISPR-Cas9 cleavage. At the other extreme, a large number of plaques (˜500 PFU, nearly the same number as the input) were produced when E. coli containing only the donor plasmid was infected (FIG. 4C). About 50 random plaques were picked from this infection and tested on E. coli B40 (sup¹) and E. coli P301 (sup⁰). Each of them was WT since they grew on both the strains (FIG. 4D). This is expected because there would be no restriction of T4 genome without the CRISPR-Cas9 plasmid. Although the donor plasmid may recombine with the delivered genome, this would be rare relative to the parental phage background. Furthermore, the donor plasmid will be degraded within minutes after T4 infection by the phage-derived denA and denB nucleases (19, 31).

Significant numbers of plaques were produced when E. coli cells containing both the CRISPR and donor plasmids were infected (˜50-fold lower than that produced on donor plasmid only) (FIG. 4C). All the 20 random plaques picked from this infection could survive only on E. coli B40 (sup¹) but not on E. coli P301 (sup⁰) (FIG. 4E), the phenotype of amber mutants. PCR-amplification and sequencing of g23 flanking the spacer sequence from individual plaques (after two rounds of plaque purification) showed that each of these plaques contained the amber mutation as well as the two additional silent mutations introduced into the protospacer sequence of the donor plasmid (FIG. 4F). These data show that the recombinant genome was selected due to restriction of the infecting genome by CRISPR-Cas9 cleavage. Hence, no significant background of WT plaques was observed. In a separate experiment, the same result have been successfully repeated using the portal protein gene (g20) by co-delivering the CRISPR-Cas plasmid containing the spacer 20-1070 (Table 1) and the corresponding donor plasmid containing the amber and silent mutations (FIG. 8A).

FIG. 8A illustrates generation of a g20 amber mutant by CRISPR-Cas editing according to one embodiment. E. coli B40 containing the spacer 20-1070 (FIGS. 2A and 2B, and Table 1) was infected with 500 PFU WT T4 according to the basic scheme shown in FIGS. 1A-1E. Plaques generated were purified, amplified by PCR and sequenced. Sequencing shows the presence of amber mutation in the edited plaque. The arrow shows the 5′ to 3′ direction of the spacer sequence marked with a blue box 802. The PAM sequence and amber mutation are marked with red box 804 and green box 806 respectively.

The above sets of data demonstrate that the T4 genome may be efficiently edited and the desired mutants may be selected with virtually no parental phage background under the strong selection pressure of CRISP-Cas9.

### Example 4

The Length of the Homologous Arms Flanking the Mutant Protospacer Sequence Correlates with Editing Efficiency

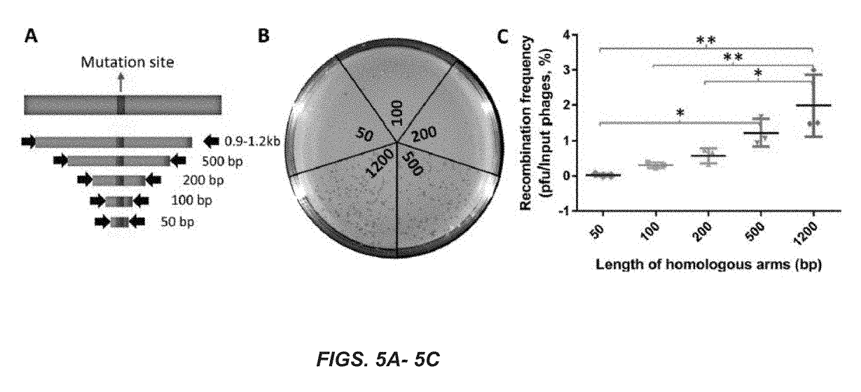

FIGS. 5A-5C illustrate effect of the length of the homologous arms on editing efficiency according to one embodiment of the present invention. FIG. 5A is a schematic of the donor templates with different lengths of homologous arms flanking the amber mutation site. FIG. 5A is an image showing plating of the phage lysates from the donor DNA templates shown in FIG. 5A. FIG. 5C is a graph showing the number of recombinant plaques produced using donor templates of different lengths. “*” and “**” indicate p<0.05 and <0.01, respectively (ANOVA).

In the above experiments in Examples 1 and 2, the amber mutation was introduced into the protospacer sequence near the center of the gene flanked by—1.2 kb DNA on either side. To determine the relationship between the length of the flanking arms and editing efficiency, five donor plasmids with different lengths of homologous arms ranging from 50 bp to 1,200 bp containing the same mutation at the center were constructed and co-transformed into E. coli B40 along with the CRISPR-Cas9 plasmid, as shown in FIG. 5A. The T4 infection assay was carried out as described above. It was found that all the donor plasmids produced plaques while there were no plaques from the control that contained only the CRISPR-Cas9 plasmid. The number of plaques increased with increasing length of the arms, as shown in FIGS. 5B and 5C. At the shortest length, 50-bp, plaques were generated at a frequency of 0.03% of the input phage, which increased up to 2-3% when the length of the arms increased to 1,200 bp (FIG. 5C). These data show that while longer homologous arms would be preferred, relatively short arms are sufficient to generate edited mutants, especially since there was no background under the CRISPR-Cas9 pressure.

### Example 5

### Genome Editing Using Two Adjacent Spacers

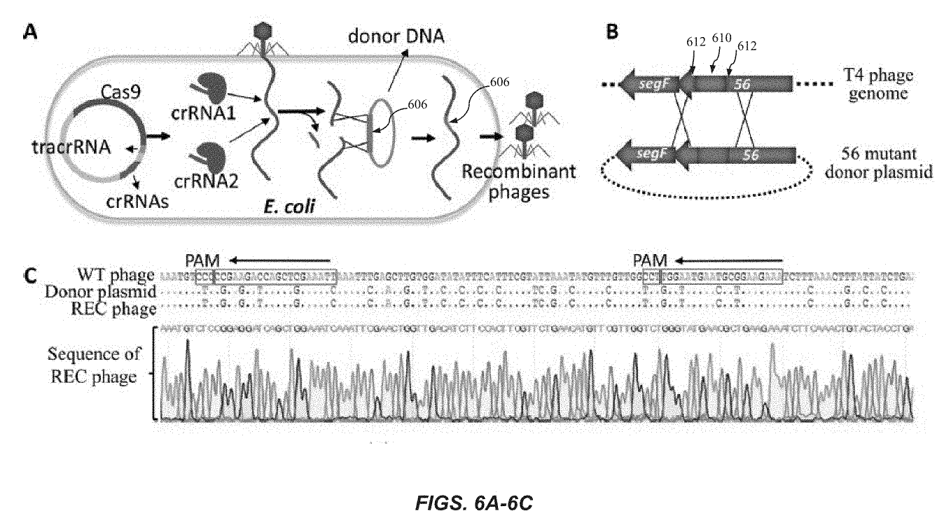

Often, it is necessary to edit the genome by introducing multiple mutations at a site or replace it with a new sequence of interest. CRISPR-Cas allows for a unique strategy to accomplish this. As shown in FIGS. 6A-6C, by expressing two crRNAs corresponding to adjacent protospacers, the CRISPR-Cas9 complex may be directed to make two cuts on the genome thereby excising the intervening sequence.

FIGS. 6A-6C illustrate exemplary genome editing using two adjacent spacers. FIG. 6A is a schematic of gene replacement by CRISPR-Cas editing using two spacers. FIG. 6B is a schematic showing protospacers in phage T4 genome are shown in portion 602 and the region targeted for replacement is shown in portion 604. The two CRISPR-Cas9 complexes formed with the two different crRNAs will create breaks in the T4 genome at the corresponding protospacer sequences in g56. This results in excision of the intervening sequence. Recombination of the genome ends with the donor DNA flanking the insert will lead to replacement of the original sequence with that present in the donor DNA (shown in pink portion 606). FIG. 6C illustrates sequencing of a random plaque showing the transfer of the new sequence into phage genome as a result of CRISPR-Cas mediated genome editing. Protospacer and PAM sequences are marked with blue box 610 and red box 612 respectively.

Recombination between the end sequences of the genome and the homologous arms of the donor plasmid replaces the excised sequence with the mutant sequence and also restores genome integrity. As shown in FIG. 6C, a recombinant plasmid containing two spacer sequences flanking a 53 nucleotide sequence was constructed such that both the crRNAs are constitutively expressed. The donor plasmid was designed by including a series of twenty-eight mutations in a sequence spanning a 112 nucleotides flanked by 500 bp homologous arms, as shown in FIGS. 6B and 6C. The donor and the CRISPR-Cas9 plasmids were co-transformed into E. coli and the T4 infection was performed as described above. Plaques at an editing efficiency of ˜1% of the input phage were generated. As shown in FIG. 6C, sequencing of one random plaque showed that the genome sequence was precisely replaced by the donor sequence.

### Example 6

Determining the Essentiality of rnlB Gene by Genome Editing

T4 genome encodes about 300 potential genes. However, despite decades of classical genetic experiments, nearly 130 of these genes still remain uncharacterized. Of these, the essentiality of several genes including the gene rnlB, an RNA ligase, remain unknown (19). The T4 RNA ligase has been extensively used in recombinant DNA technology to ligate single stranded RNA (or DNA) molecules (32).

T4 produces two RNA ligases encoded by genes 63 and rnlB (32, 33). While 63 RNA ligase I is essential in E. coli strains containing the prr locus (33), the importance of rnlB RNA ligase II is unknown. In E. coli strains containing the prr locus, the tRNA^{Lys} is cleaved 5′ to the wobble position by an anticodon nuclease (suicide function) induced upon T4 phage infection. The g63 RNA ligase together with the T4 polynucleotide kinase (pnk) repairs the tRNA allowing productive phage infection (34). If Pnk and RNA ligase I are not present, the synthesis of viral proteins and phage production is impaired by depletion of tRNA^{Lys} (34). Whether the rnlB RNA ligase II also plays a role in phage survival is unknown (34).

To determine if the above CRISPR-Cas9 T4 genome editing strategy could be used to determine the essentiality of rnlB gene, an rnlB spacer plasmid (rnlB270, Table 1) and a donor plasmid with an amber codon at amino acid 98 were first constructed. The plasmids were co-transformed into E. coli and plaques were selected using the same experimental design described in FIG. 4A-4F. The sequencing data showed that the T4 genome was successfully edited by transferring the amber mutation from the donor plasmid into the CRISPR-Cas9 cleaved T4 genome. Spot testing of individual edited plaques showed that the rnlB amber phage may survive both on E. coli CR63 (sup¹) and B834 (Sup⁰), indicating that the rnlB gene is not essential for phage T4 viability under the laboratory conditions used to grow the phage. Since an amber mutation may spontaneously revert at some frequency, it would be necessary to delete a portion of the gene to completely knock out function. Further, an ability to create deletion mutants would be critical to engineer phage genomes for phage therapy applications where background would not be tolerated.

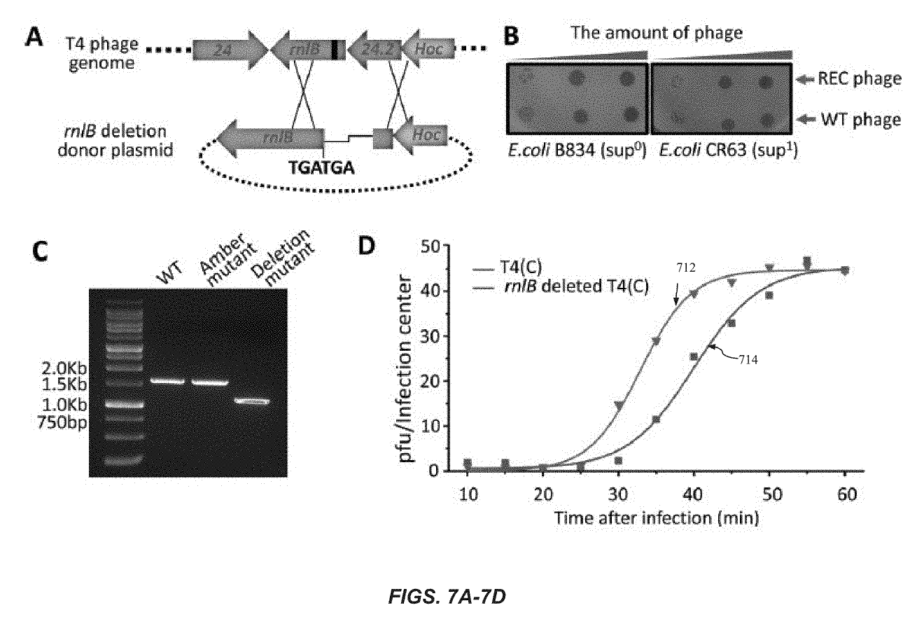

FIGS. 7A-7D illustrate the phage T4 rnlB being a nonessential gene. FIG. 7A is a schematic showing the introduction of a 437-bp deletion into rnlB region using CRISPR-Cas9 genome editing. Protospacer is shown in black portion 702 and Hoc gene is shown in orange portion 704. Two stop codons were also introduced following the deletion. The line shows the deleted region. FIG. 7B is a set of images showing spot-testing of an rnlB deletion mutant plaque, showing that it grows on both E. coli B834 (sup⁰) and CR63 (sup¹). WT phage was used as a control (blue arrow, which is the lower arrow). FIG. 7C is an image of PCR demonstrating that the rnlB mutant plaque has a deletion of appropriate size. FIG. 7D is a graph showing one step growth curve of rnlB deleted T4(C) (curve 712) and WT T4(C) phages (curve 714) on E. coli DH5a.

As shown in FIGS. 7A-7D, to knock-out the rnlB gene, a deletion donor plasmid was constructed, in which a 437-bp fragment containing the last 143-bp corresponding to the 3′-end of gene 24.2 and the 294-bp of rnlB gene and its upstream region were deleted. Consequently, the DNA sequence corresponding to the promoter, the translation initiation site, and the first 98 amino acids of rnlB gene were removed. Two stop codons were also introduced right before the amino acid 99 to further prevent any run-on translation from an upstream sequence, as shown in FIG. 7A. The donor and CRIPSR-Cas9 plasmids (24.2-181, Table 1) were co-transformed into E. coli strains B834 (sup⁰), which was then infected by phage T4(C). Five progeny plaques were randomly picked and tested for their ability to grow on E. coli strains CR63 (sup¹) and B834 (sup⁰). All the plaques survived on both the E. coli strains and the representative results of spot tests are shown in FIG. 7B. The recombinant phages were plaque-purified and the mutated region was amplified using flanking primers rnlBFW and 24.3XbaBW with appropriate controls. The amber mutant from above and the WT T4 had the same size PCR product, ˜1.5 kb, whereas the deletion mutant phage had ˜400 bp shorter band demonstrating adeletion in the rnlB gene of T4 genome, as shown in FIG. 7C. A one-step growth experiment was performed with the rnlB.del phage and compared with the WT phage. The data showed that the rnlB deletion mutant generated a similar burst size of progeny phage as the parental WT T4(C) but the eclipse time is about 5 minutes shorter, as shown in FIG. 7D. These results demonstrate that the T4 rnlB gene is not essential for phage T4 infection under the laboratory conditions.

### Example 7

### Discussion 1

Phage T4 is one of the most well characterized viruses. The atomic structures of essentially all the key components of the virus including the head, tail, fibers, and the DNA packaging machine have been determined (2, 35-40). Genetic and biochemical pathways were elucidated in 60's and 70's that revealed common principles of virus assembly (19). Combined with the unique features of the T4 outer capsid proteins Hoc and Soc and the promiscuous nature of the DNA packaging machine, a platform to deliver genes and proteins into mammalian cells has been developed (12, 13, 41). However, it has been difficult to engineer the T4 genome owing to its modified genome that is refractory to most restriction enzymes (19, 27). Lack of a clustered nonessential region in the genome that may be replaced with foreign DNA posed another barrier to use T4 as a cloning or protein/gene delivery vector (20). Overcoming such barriers would be essential to unleash the potential of T4 and other phages for biomedical applications. Our studies reported here demonstrate that some of these barriers could be overcome by CRISPR-Cas genome editing, which could potentially be extended to phages in general.

Studies disclosed herein led to several new findings. First, the infection data from twenty-five different spacers spanning the T4 genome show that the WT phage T4 genome containing the ghmC-modified DNA is vulnerable to CRISPR-Cas9 attack. However, it is not as highly susceptible to Cas9 cleavage as the T4(C) mutant genome containing the unmodified C-DNA. While the T4(C) mutant phage genome shows very low plating efficiency, on the order of ˜10⁻⁵ of the input phage, the plating efficiency of the WT T4 phage varies greatly, between 10⁻¹ to 10⁻⁶ of the input phage. Preliminary sequencing of the plaques that arose in WT or T4(C) mutant infections shows CRISPR-Cas escape mutations in the PAM trinucleotide sequence or the protospacer sequence. However, more work is underway to delineate the mechanisms involved in the selection of escape mutants.

Second, data of the present disclosure demonstrate that CRISPR-Cas9 cleavage allows the selection of edited T4 genomes. This required co-introduction into the same E. coli cell of both the CRISPR-Cas spacer plasmid and a donor plasmid containing the desired mutation(s). Conveniently, these mutation(s) also result in mismatch between the spacer-derived crRNA and the protospacer sequence of the donor, thereby sparing the donor plasmid from cleavage by Cas9 nuclease. This allows recombination between the cleaved ends of the delivered genome and the donor sequence. That such recombinants arose at high frequency (up to 2-3% of the input) means that the ends of the Cas9 cleaved genome remained competent for recombination and not degraded by nucleases. Whether this rescue is carried out by the E. coli recombinase or the highly efficient phage T4 recombination system (ref) (42) requires further investigation. A notable aspect of the CRISPR-Cas mediated recombinational editing is that there is essentially no parental phage background among the progeny phage, presumably due to the strong pressure of CRISPR-Cas9 nuclease which selects out the parental background.

Third, the T4 genome editing strategy may be extended to constructing complex mutants involving multiple point mutations, insertions, and deletions, including the simultaneous use of two spacers. The frequency of survived mutant progeny decreases with increasing length of the mutant region and increases with increasing length of the flanking homologous arms. A limitation of the strategy, however, is that the editing site must be associated with the PAM sequence, 5′-NGG-3′, for the CRISPR-Cas9 complex to recognize the target and cleave the adjacent spacer sequence. However, this may be overcome by using mutant Cas9 proteins that exhibit increased breadth of the PAM recognition sequences (43).

Finally, the CRISPR editing is demonstrated to allow functional characterization of the phage genome, especially of the nonessential genes, that has been otherwise difficult by the classical genetic strategies (44). A 437-bp deletion is readily introduced into the T4 RNA ligase II gene rnlB using CRISPR-Cas editing. The rnlB knock-out mutant not only forms plaques but also that its burst size is similar to that of the parental phage. However, the knock-out phage exhibits a shorter eclipse time compared to the parental phage the reason for which is not clear. These data suggest that the RNA ligase function of rnlB is not essential for phage infection, at least under the laboratory growth conditions at 37° C. However, it is possible that rnlB might provide survival advantage(s) under certain E. coli genetic backgrounds, or in the absence of g63 RNA ligase I, which also has a second essential function, attachment of tail fibers to the baseplate (45). The edited mutants such as the rnlB phage now allow detailed characterizations of the complex functional and evolutionary relationships among the phage genes as well as the host-virus interactions.

In conclusion, our studies for the first time established a CRISPR-Cas editing strategy to engineer both modified and unmodified genomes of phage T4. Selection of edited mutants under the strong selective pressure of CRISPR-Cas9 nuclease makes it a powerful strategy to modify phage T4 genome for functional characterizations as well as to accelerate the development of T4 as a gene/protein delivery vehicle. This editing strategy could potentially be applied to other phage genomes to harness the vast potential of the naturally occurring phages for various biotechnology applications and phage therapies.

### Example 8

Partial Resistance of ghmC-Modified DNA to CRISPR-Cas9 Drives the Evolution of Phage T4 Genome

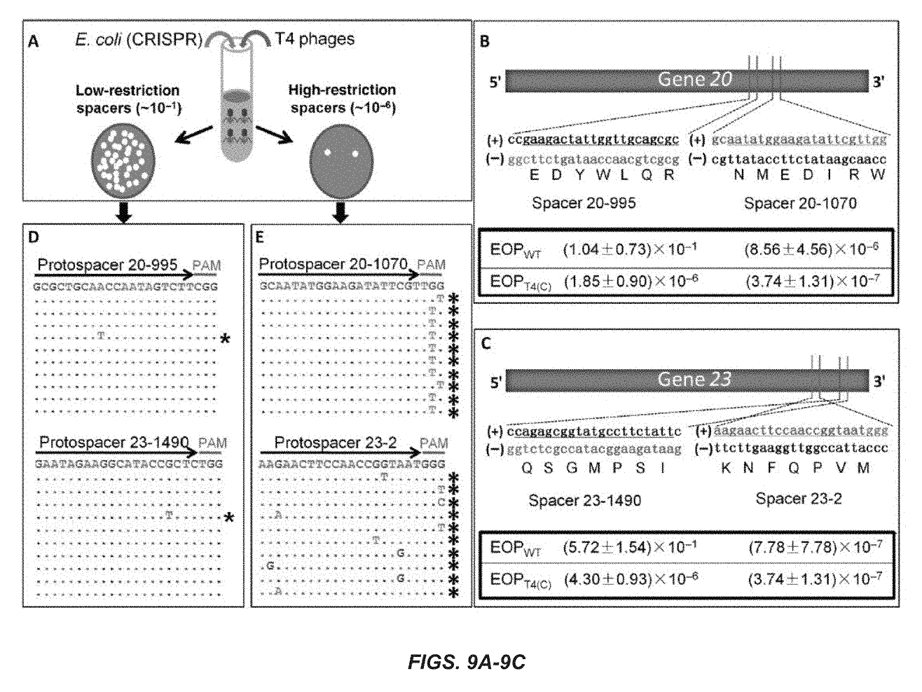

FIGS. 9A-9E illustrate the evolution of phage T4 genome under CRISPR-Cas9 pressure. FIG. 9A is an experimental scheme for testing the effect of CRISPR-Cas on phage T4 infection according to one embodiment. Efficient cleavage by Cas9 nuclease at the protospacer sequence disrupts the phage genome resulting in loss of plaque-forming ability (plate on the right; high restriction spacer). Inefficient cleavage by Cas9 nuclease reduces plating efficiency (plate on the left; low efficiency spacer). FIGS. 9B and 9C illustrate plating efficiencies of high restriction spacers 20-1070 and 23-2, and low restriction spacers 20-995 and 23-1490. Shown are the locations of spacers on genes 20 and 23, and the nucleotide and amino acid sequences corresponding to the protospacer (red) and PAM (green) sequences. The sequences of the complementary strand are shown in black. Efficiency of plating (EOP) was determined as described in Materials and Methods. The data shown are the average of three independent experiments ±SD. FIGS. 9D and 9E illustrate the alignment of sequences corresponding to single plaques produced from infection of various spacer-expressing E. coli. The DNA from single plaques was amplified and sequenced as described in Materials and Methods. The black arrows correspond to the spacer sequences and the red lines correspond to the PAM sequences. The sequence at the top of each panel corresponds to the WT sequence. The dotted lines below correspond to the sequence obtained from each plaque. Only the mutated nucleotides are shown. * indicates the mutant sequences.

As disclosed above, twenty-five spacer sequences across the T4 genome have been screened for their ability to restrict the WT T4 or the T4(C) mutant phage infection of E. coli bacteria containing the S. pyogenes type II CRISPR-Cas9 system (55). All the components of the system; crRNA, tracrRNA, and Cas9 nuclease were constitutively expressed from a resident plasmid under the control of appropriate promoters (see Materials and Methods). Although the Cas9 nuclease is not native to E. coli, it is one of the best defined models to analyze how phages respond to CRISPR-Cas attacks by the bacteria. The WT phage infections that deliver ghmC-modified genome, not surprisingly, produced more plaques when compared to the T4(C) mutant phages that deliver the unmodified cytosine (C) genome (FIGS. 9B and 9C). This difference may be due to more frequent escape of the ghmC-genome from cleavage by the Cas9 nuclease. Strikingly, however, the differences in plating efficiencies between the WT and T4(C) mutant phages, within even the same gene, varied vastly, up to 5-6 orders of magnitude (FIGS. 9A-9C). For instance, spacers 23-2 and 20-1070, both in the essential genes coding for the major capsid protein gp23 and the portal protein gp20, respectively, were highly restrictive (high restriction spacers). The plating efficiency was ˜10⁻⁶-10⁻⁷ for both the WT and T4(C) phages. On the other hand, two other spacers in the same genes, 23-1490 and 20-995, showed high level of restriction for the T4(C) phage (˜10⁻⁶) but poor restriction for the WT phage (˜10⁻¹) (low restriction spacers). This was intriguing because no obvious differences in the spacer sequences, such as the C or GC content that could affect Cas9 cleavage, or the location of the spacer in the coding versus non-coding strand, could explain this difference. Further investigation is necessary to determine the underlying mechanism.

Sequencing of the CRISPR-resistant plaques (CRPs) from the high restriction spacers showed that 100% of the plaques have mutations in the PAM or protospacer sequence (FIG. 9E). Clearly, these represent rare pre-existing mutations present in the phage stocks that either prevented binding of CRISPR-Cas to the PAM sequence or cleavage of the protospacer sequence by the Cas9 nuclease, thus escaping the CRISPR surveillance. On the other hand, sequencing of CRPs from the low restriction spacers showed no mutations in PAM or protospacer sequences. This was consistent with our hypothesis that resistance here was not due to a mutation but due to escape of the WT ghmC-modified genome from Cas9-mediated gene disruption due to poor cleavage. Surprisingly, however, it is found that although most of the plaques have WT protospacer sequence, ˜1 in 10-20 (5-10%) have mutations in the protospacer or PAM sequence (FIG. 9D). This was observed in both g20 and g23 with the low restriction spacers. This was completely unexpected because a mutation frequency of ˜10⁻¹ is too high to be due to pre-existing mutations which is expected, and determined (FIGS. 9B and 9C), to be on the order of ˜10⁻⁷ (56, 57). No such mutations were observed in the control plaques generated without the CRISPR-Cas9 pressure. Therefore, the high mutation rate of WT ghmC-modified T4 phage in CRISPR background must be due to rapid evolution and selection of mutants during active replication of phage genome in the infected cell under the pressure of CRISPR-Cas9.

### Example 9

### A Model for Rapid Evolution of Phage T4 Genome Driven by CRISPR-Cas

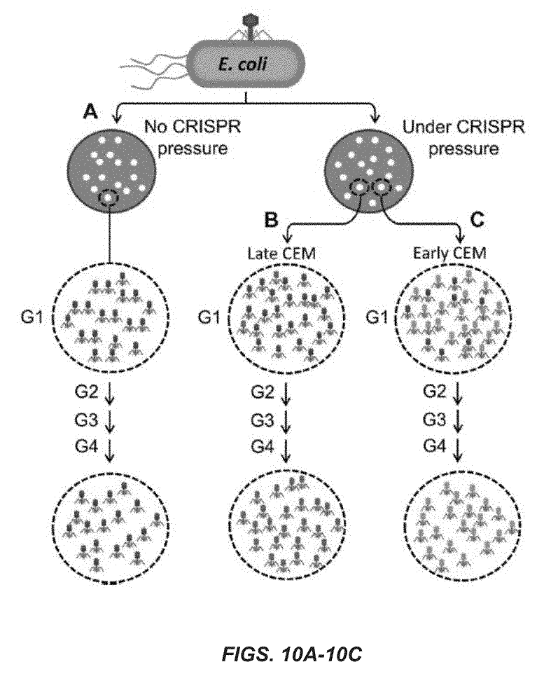

FIGS. 10A, 10B, and 10C illustrate a model for CRISPR-Cas9 driven evolution of phage T4 genome according to one embodiment of the present invention. Schematic depicting the patterns of phage progeny in single plaques starting from a single phage infecting a single E. coli cell. Details of the model are described in Results.

A model for evolution of phage mutants under the pressure of CRISPR-Cas9 (FIGS. 10A, 10B, and 10C) is proposed. At a multiplicity of infection (m.o.i.) of 0.001 (FIG. 9A), each plaque originates from infection of a single E. coli bacterium with a single WT phage. For a low restriction spacer, about 10-20% of the genomes escape CRISPR-Cas9 cleavage (see FIGS. 9B, 9C) and enter phage replication cycle triggering the production of new genomes. However, the constitutively expressed CRISPR-Cas components from the CRISPR plasmid may cleave the newly replicated genomes, albeit inefficiently (55). Although, continued production of CRISPR-Cas9 components may cease because early expression of T4 phage nucleases denA and denB degrade the CRISPR plasmid (58). The CRISPR-cleaved DNA may initiate new replication events because phage T4 has no defined replication origin and its replication is largely initiated by the recombination dependent invasion of DNA ends into the actively replicating DNA (59-62). In addition, since T4 is a highly recombinogenic phage expressing potent recombination and repair enzymes (19), the ends may be repaired by a combination of mechanisms involving these enzymes, mechanisms phage T4 uses for its own genome replication to generate a massively branched concatemeric DNA network. Consequently, the cleaved protospacer sequences might create hotspots for mutation as the T4-infected CRISPR-E. coli continually accumulates concatemeric DNA containing the repaired genomes of CRISPR-Cas cleaved DNA, which would then be encapsidated generating a burst of progeny (36, 37).

A plaque represents a locus where a series of phage infection cycles productively lyse E. coli bacteria and concentrate ˜10⁷ progeny phages (FIGS. 10A, 10B, and 10C). Spontaneous (random) mutants do exist in this population by classical error-prone replication mechanisms but at a very low frequency, roughly on the order of ˜10⁻⁶ to 10⁻⁷ (56). Under CRISPR pressure (FIGS. 10B and 10C), however, if a CRISPR escape mutant (CEM) arose from repairing Cas9-cleaved ends as described above, that mutant phage may have greater fitness as it is no longer cleaved by Cas9 nuclease. Hence, it may produce greater number of progeny viruses compared to the WT phage that has been partially restricted by Cas9. Thus, in subsequent generations, the fraction of CEM phages may raise dramatically. Consequently, a plaque produced under CRISPR-Cas9 pressure may consist of a mixture of WT and CEM phages (G1 in FIGS. 10B and 10C) as opposed to a plaque produced without the CRISPR-Cas pressure (FIG. 10A). However, the fraction of CEM phages in a given G1 plaque may depend on the time at which the CEM arose. If it arose late after the initial infection (FIG. 10B), the plaque may predominantly have WT phages and few CE mutants. But if it arose soon after the initial infection (FIG. 10C), CEM progeny may accumulate rapidly in the subsequent generations and predominate the population in the plaque. Consistent with this model, the CEMs were found at a remarkably high frequency among the first generation G1 plaques under CRISPR-Cas pressure, ˜5% for the low restriction g20 spacer 20-995 and ˜10% for the low restriction g23 spacer 23-1490 (FIG. 9D). In contrast, the CEM mutation frequency for the high restriction spacers in the same genes was ˜10⁻⁶ to 10⁻⁷, similar to the expected spontaneous mutation frequency (FIGS. 9B and 9C).

### Example 10

### Selection for CRISPR-Driven Mutations in the Portal Protein Gene

FIGS. 11A-11F illustrate selection of CEMs in the portal protein gene. FIG. 11A shows that plaques produced from WT phage T4 infection of CRISPR-E. coli DH5α expressing spacer 20-995 (G1) were transferred to a fresh plate and the process was repeated (G2-G4). The DNA from single plaques was amplified and sequenced. The panels on the left show alignment of sequences in the same manner as described in legend to FIGS. 9A-9C. FIG. 11B illustrates that the mixture of phages from a G3 plaque was separated by serial dilution and single plaques produced by individual variants were sequenced as shown in FIG. 11C. FIGS. 11D and 11E illustrate a determination of the relative fitness. The same phage mixture was used to infect E. coli DH5α expressing spacer 20-995 in a liquid culture. Single plaques obtained from the progeny produced after 315 min infection were picked and sequenced. The percentages of each CEM in the starting sample of evolution is shown in pie chart of FIG. 11E and the percentage of each CEM in the sample after 315 min of evolution is shown as pie chart in FIG. 11F. The spacer and PAM sequences are marked with arrow and red lines 1102, 1104, and 1106, respectively. See Materials and Methods for details.

Two tests to evaluate the above model were applied. First, if the model is correct, then every WT phage infection, thus every plaque that arises as a result on CRISPR-E. coli containing a low restriction spacer, should be on a trajectory to evolve into a CEM plaque. To test this prediction, each G1 plaque was transferred to a fresh CRISPR-E. coli lawn and allowed to form second generation (G2) plaque (FIG. 11A). This was repeated up to five generations (G3 to G5). Individual plaques from G2 to G5 were picked and the DNA flanking the protospacer/PAM sequence was sequenced (FIG. 11A). The data showed that the frequency of CEMs dramatically increased from 10% in G1 plaques to 50% in G2 plaques, and to 90% and 100% in G3 and G4 plaques, respectively. Furthermore, each plaque went through its own evolutionary trajectory both in time and sequence, selecting different CE mutations, although some of the mutations were repeatedly selected. Furthermore, 100% of the CEMs are in the protospacer/PAM sequences. No CEMs were found in any of the control G1 to G5 plaques that were not under the CRISPR-Cas pressure.

The second test was to capture an intermediate state of the evolutionary process. The disclosed model predicts that, at an intermediate stage, a single plaque may contain more than one CEM mutant phages plus the WT phages but eventually, the most-fit mutant phage(s) under CRISPR-Cas9 pressure predominate the population. To capture this state, a G3 plaque that showed significant background in the sequencing chromatogram at certain positions of the PAM/protospacer sequence is selected. This indicated the presence of a mixture of sequences. Individual phages present in this plaque were separated by serial dilution and plated on E. coli without the CRISPR-Cas pressure to ensure that no further evolution occurred (FIG. 11B). Of the 10 progeny phages sequenced, one had a WT sequence, six had mutations changing a G to an A of the two strictly required Gs of the 5′-NGG-3′ PAM recognition sequence, two had C to T mutations in the protospacer sequence, and one had an A to G mutation also in the protospacer sequence (FIG. 11C). This pattern demonstrated independent evolution of different CE mutants within the same plaque and near disappearance of the WT phage.

To determine the relative fitness of these CEMs, this G3 phage mixture was then used to infect the CRISPR-E. coli at a low m.o.i (0.001) and allowed to grow for several hours (FIG. 11D). The progeny phages were plated and single plaques were isolated and sequenced. The data showed that, although there were five different variants in the starting mixture of the G3 plaque (FIG. 11E), only two CE mutants were recovered after several generations (FIG. 11F). Of these two, the C to T mutation in the protospacer sequence (silent mutation) predominated with 70% of phages in the progeny population whereas the minor variant (30% of the progeny) had the G to A missense mutation (Thr to Ile) in the PAM sequence (FIG. 11F).

The above sets of data confirm the basic predictions of the proposed CRISPR-driven evolution of the phage T4 genome (FIGS. 10A, 10B, and 10C) disclosed herein.

### Example 11

### CRISPR-Driven Evolution of the Major Capsid Protein Gene

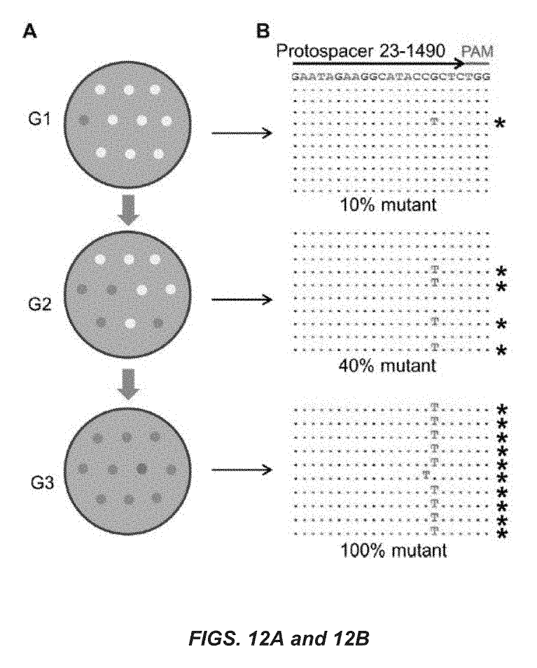

FIGS. 12A and 12B illustrate selection of CEMs in the major capsid protein gene. FIG. 12 is an experimental scheme for analysis of CEM selection in the major capsid protein gene, which is the same as that used for the portal protein gene. Panels in FIG. 12B show the sequences of CEMs. See Materials and Methods and legend to FIG. 11A for details.

To test if the CRISPR-driven evolution is applicable to any other (essential) gene in the phage T4 genome, the above analyses were carried out for another low restriction spacer 23-1490 which is part of the major capsid protein gene 23 (FIGS. 9A-9E). The data demonstrated the same pattern (FIGS. 12A and 12B); the CEMs arose at a frequency of 10% among G1 plaques, which increased to 40% in G2 plaques and to 100% in G3 plaques. However, the pattern of the CEM phage selection in this protospacer region was different from that of the portal protein protospacer described above. This is expected because the mutations that may restore the major capsid function would be different from that of the portal protein. Here, only two types of CEMs were selected, a predominant G to T mutation (9 out of 10 plaques) and a minor C to T mutation (1 out of 10 plaques), both in the protospacer region. No mutations in the PAM sequence were recovered.

### Example 12

### CRISPR-Escape Mutants Exhibit Dual Phenotype

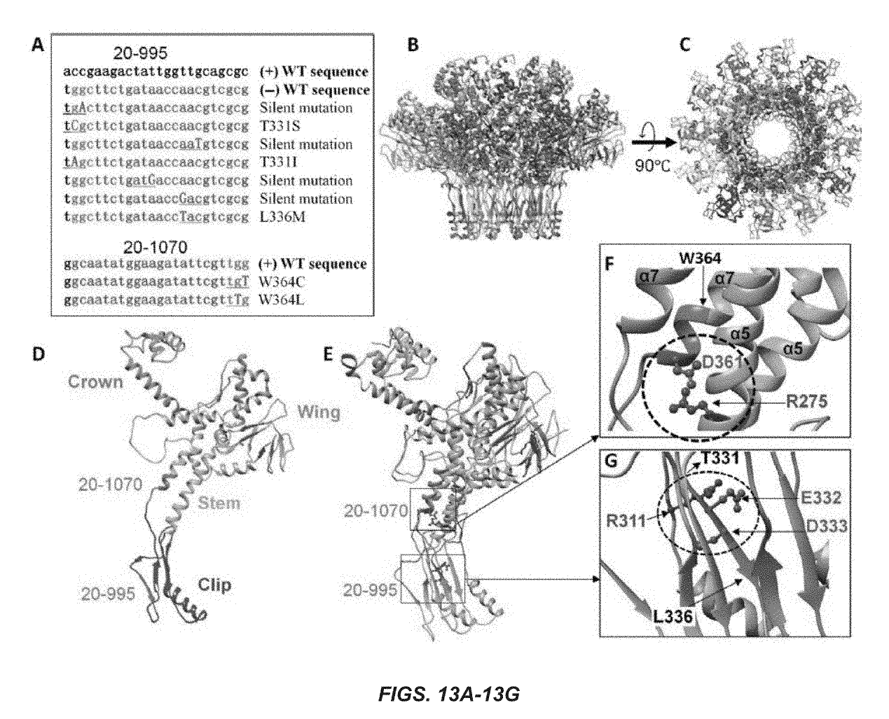

FIGS. 13A-13G illustrate the characteristics of the CEMs obtained from gene 20 spacers. FIG. 13A is a list of CEM sequences obtained from gene 20 spacers. The spacer sequence and PAM are shown in red and green, respectively, and the mutated nucleotides are shown in blue (bold) and underlined. The WT sequences are shown at the top of each alignment. FIGS. 13B-13G illustrate structural analysis of the CEMs of the portal protein gp20. FIG. 13B is a side view and FIG. 13C is a top view of the structure of the dodecameric gp20 portal assembly with each subunit shown in different color. FIG. 13D show a single subunit of gp20 and FIG. 13E show two subunits of gp20. The single subunit of gp20 shown in FIG. 13D and the two subunits of gp20 shown in FIG. 13E show: i) the critical salt bridge between the D361 residue of one subunit and R275 residue of an adjacent subunit (circled), shown in FIG. 13F, and ii) the functionally important residues of the clip domain, E332 and D333 of one subunit forming a salt bridge with R311 of an adjacent subunit (circled), shown in FIG. 13G. The regions corresponding to the protospacer and PAM sequences of spacers 20-1070 (shown in FIGS. 13D, 13E, and 13F) and 20-995 (shown in FIGS. 13D, 13E, and 13G) are shown in magenta color. The positions of the mutated residues of the CEM phages corresponding to spacers 20-1070 (W364) (FIG. 13F) and 20-995 (T331 and L336) (FIG. 13G) are shown with arrows.

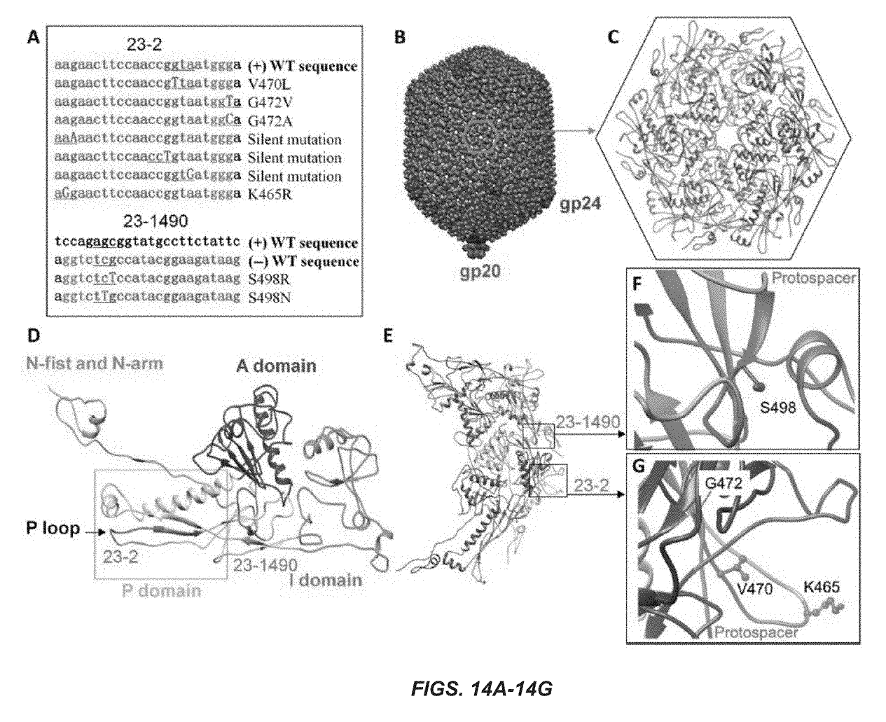

FIGS. 14A-14G illustrate the characteristics of the CEMs obtained from gene 23 spacers. FIG. 14A is a list of CEM sequences obtained from gene 23 spacers. The spacer sequence and PAM are shown in red and green, respectively, and the mutated nucleotides are shown in blue (bold) and underlined. The WT sequences are shown at the top of each alignment. FIGS. 14B-14G illustrate structural analysis of the CEMs of the major capsid protein gp23. FIG. 14B is a side view of the phage T4 capsid. FIG. 14C is a top view of the hexameric gp23 capsomer. FIG. 14D shows a single subunit of gp23. FIG. 14E shows three subunits of gp 23. The single subunit, as shown in FIG. 14D, and the three subunits of gp23, as shown in FIG. 14E, are involved in a network of inter-subunit interactions. The regions corresponding to the protospacer and PAM sequences of spacers 23-1490 are shown in magenta color in FIGS. 14D, 14E, and 14F and the protospacer and PAM sequences of spacers 23-2 are shown in magenta color in FIGS. 14D, 14E, and 14G. The side chains of the mutated residues of the CEM phages corresponding to spacers 23-1490 (S498) (FIG. 14F) and 23-2 (K465, V470, and G472) (FIG. 14G) are shown in red.

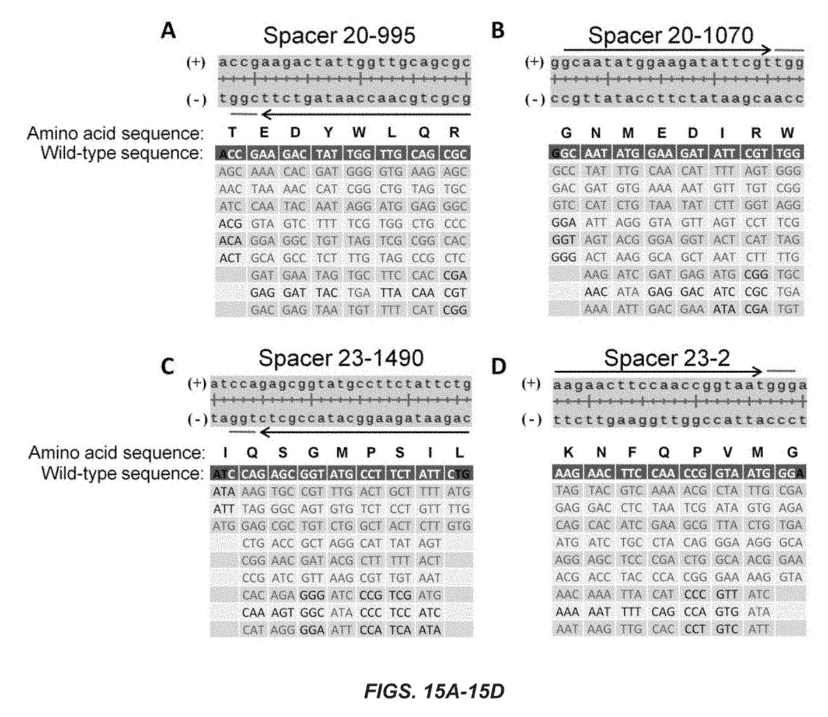

FIGS. 15A, 15B, 15C, and 15D are lists of all possible single mutations in each spacer that retain the reading frame. The spacer sequences are shown at the top of each panel. The spacer and PAM sequences are shown with arrows and red lines respectively. The possible single mutations at each codon of the spacers 20-995 (FIG. 15A), 20-1070 (FIG. 15B), 23-1490 (FIG. 15C), and 23-2 (FIG. 15D) are shown below the wild-type sequence in each table. The silent mutations are shown in black color and the missense mutations in red color.

FIGS. 16A, 16B, and 16C illustrate a relative fitness of the CEMs that escaped Cas9 cleavage of the protospacer 20-995 of the portal protein gene. Four phage mutants with nucleotide changes G991 to A (Thr to Ile), G992 to A (silent mutation), A1005 to G (silent mutation), and C1007 to T (silent mutation), were mixed in equal ratios and used to infect E. coli DH5α with or without the CRISPR-Cas9 plasmid (FIGS. 16A and 16B). Three hundred and fifteen min after infection, the progeny phages were collected and plated on LB plates after serial dilution. Twenty single plaques were picked, PCR-amplified, and sequenced (FIG. 16C). The spacer sequence and PAM are marked with arrow and red line respectively. The percentages of each CEM in the starting sample and after 315 min evolution are shown in the pie charts of FIGS. 16B and 16C.

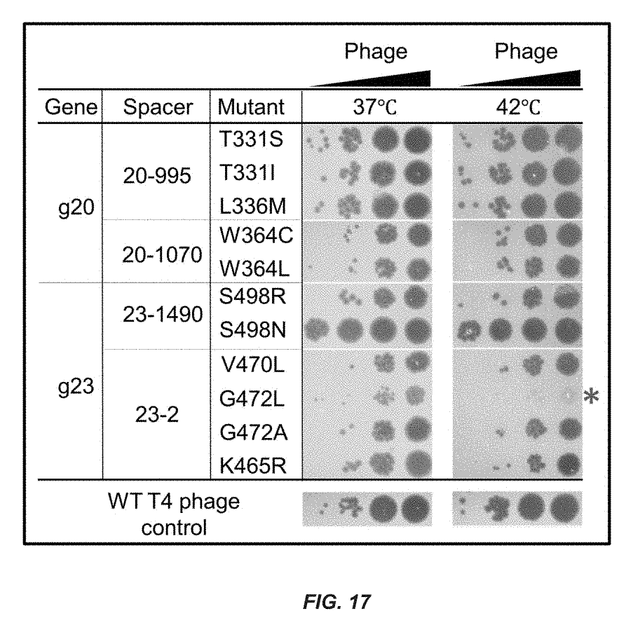

FIG. 17 illustrates the temperature sensitivity of the CEMs containing amino acid changes according to one embodiment. The mutant phages were spotted on E. coli DH5α lawn for lytic growth at 37° C. or 42° C. The amino acid changes and their location in genes 20 or 23 are shown. * indicates the heat-sensitive phenotype of the G472L CEM in g23.

In this example, forty CRISPR-escape mutations were isolated from either the high restriction spacers or the low restriction spacers. Of these, eighteen were unique variants and the rest were repeat isolates of one of the variants (FIGS. 13A-13G and FIGS. 14A-14G). All were single point mutations, each retaining the reading frame as required to express the essential gene functions of the major capsid protein and the portal protein. Seven of the mutations were silent and eleven involved amino acid changes. The four spacer regions contain 33 amino acid codons which include a total of 276 possible single point mutations while maintaining the reading frames of the essential genes 20 and 23 (FIGS. 15A, 15B, 15C, and 15D). Of these, forty-seven or 17% would be silent mutations and the rest would be missense amino acid substitutions. However, since the percentage of recovered silent mutations was—39% (7/18), more than twice that of what would be expected if the mutations were evenly distributed between the silent and missense mutations, it appears that the selection of CEMs is biased towards silent mutations. This might be because some (many) of the amino acid changes cost in fitness because these phage structural proteins are critical for head assembly (2, 13) and genome packaging (38). This was evident in at least one instance; when a cocktail of five CEM phages as present in a single plaque was used to infect E. coli, the C to T silent CEM at nucleotide 1008 of the protospacer sequence was preferentially selected (FIG. 11E, 11F). When this experiment was repeated slightly differently, by mixing the variants in equal proportion at the start (FIGS. 16A and 16B), again this and another silent mutation were recovered at greater frequency whereas the CEM with an amino acid change (Thr to Ile) became “extinct” after a few hours of growth (FIG. 16C). However, as the following data show, selection of the CEMs was spacer-specific and exhibited different patterns, in part depending on the functional importance of the amino acid sequence encoded by the protospacer sequence.

In the case of the low restriction spacer 20-995, in addition to four silent CEMs, three mutants with amino acid changes were recovered (FIG. 13A). The amino acid sequence ₃₃₁TEDYWLQR₃₃₈ (SEQ ID NO: 26) corresponding to the 20-995 protospacer encodes a 3-strand in the “clip” domain of the portal protein (FIGS. 13B-13E). It is part of the hydrophobic core of the domain and in addition, contains two negatively charged residues E332 and D333 that form a salt bridge with R311 of another β-strand of an adjacent subunit. Our mutational studies show that the salt bridge is critical for function. Consistent with the importance of this β-strand, the CEMs selected in this protospacer sequence fall at sites flanking the strand, the residues T331 (T331S and T331L) and L336 (L336M) (FIG. 13G).

For the high restriction spacer 20-1070, only two CEMs with changes at the same amino acid, W364C and W364L, were repeatedly selected (FIG. 9E and FIG. 13A). This is also consistent with our functional data in that the amino acid sequence encoded by this protospacer sequence (₃₅₇GNMEDIRW₃₆₄ (SEQ ID NO: 27)) is critical for head assembly and DNA packaging (63) (FIGS. 13B-13F). The amino acid residues 361-374 form the helix α-7 which, together with helix α-5 form the “stem” domain that lines the ˜35 Å diameter central channel of the dodecameric portal vertex (63). The channel allows passage of DNA into the capsid during packaging and out of the capsid during infection. The D361 residue of the α-7 helix forms a critical inter-subunit salt bridge with R275 residue of α-5 helix of an adjacent subunit (FIGS. 13D and 13F). This inter-digitation stabilizes the dodecameric portal structure and is essential for head assembly (63). Combinatorial mutagenesis of this region shows that no substitutions are tolerated at D361. The CEM selection confirmed this point as no mutations were recovered at or near D361. The CEM approach, however, seems more powerful because it allowed rapid scanning of seven amino acids spanning the protospacer sequence in one experiment and further identifying that the W364 residue that is upstream to D361 may be substituted without losing function.

Different CEMs were selected for the g23 spacers that also included both silent mutations and amino acid substitutions in the protospacer and PAM sequences (FIG. 14A). The low restriction spacer 23-1490 encodes amino acids ₄₉₇QSGMPSIL₅₀₄ (SEQ ID NO: 28) that links the axial domain (A-domain) to the peripheral domain (P-domain) whereas the high restriction g23 spacer 23-2 comprises of the amino acid sequence ₄₆₅KNFQPVMG₄₇₂ (SEQ ID NO: 29) that are part of the P-loop sequence (64) (FIGS. 14B-E). The A-domain through inter-subunit interactions is responsible to assemble hexameric capsomers whereas the P-domain and the P-loop residues are important for interface interactions between capsomers (64). Consistent with our structural analyses(64), the recovered CEMs correspond to residues that don't appear to be involved in these interactions. For instance, the side chain of S498 residue at which two CEMs were recovered (S498N, R) is fully exposed and not in proximity to any other side chains within ˜5 Å distance (FIG. 13F). The CEM G472L at the base of the P-loop resulted in a heat-sensitive phenotype probably because it affected the interaction of the P-loop with the “insertion” domain (I-domain) linker of the adjacent subunit (FIGS. 14E and 14G and FIG. 17).

The above sets of data suggest that the selection of CEMs was driven not by whether the spacer is of low or high restriction type, or silent vs amino acid change, but rather by their ability to overcome two strong selection pressures, i) resistance to Cas9 nuclease and ii) retaining essential phage function. Although the sample size of the mutants analyzed here is small, it seems clear that the CRISPR-Cas selection approach can be used to generate pools of CEMs, the analysis of which may generate a detailed functional map and reveal the mechanistic requirements for a given phage function or for Cas9 cleavage. These are currently under investigation.

### Example 14

### Discussion 2

CRISPR-Cas is generally thought of as an adaptive immune system that has evolved to protect the bacterial host against phage infections which are often lethal (49). An unexpected finding of this study is that the CRISPR-Cas might be a double-edged sword, not only a defensive mechanism against phages but also, a potentially robust platform for phage evolution, which would ultimately benefit both the host and the virus.

The surprising observation was that mutations accumulated in phage genome at unusually high frequency and rapidity among the progeny produced from CRISPR-Cas9 E. coli infected with WT T4 phage containing the ghmC-modified genome. Virtually every such infection was found to be on an evolutionary trajectory to become CRISPR-resistant, with the mutations clustering exclusively in the protospacer and PAM sequences. These CEMs outcompeted the WT phage and predominated the population even among the first generation plaques, about 5-10% of them, which increased to 40-50% in the second generation and nearly 100% in the third generation. These frequencies are striking, about 6 orders of magnitude greater than the spontaneous mutation frequency, which is on the order of ˜10⁻⁷ (56, 57). All the CEMs exhibited dual phenotype, resistance to CRISPR-Cas9 and retention of the respective gene function. This seems to be a general pattern as it was observed with two essential phage structural genes, one coding for the major capsid protein gp23 and another for the portal vertex protein gp20.

That such high mutation frequency was observed with the low restriction spacers (most of the spacers) suggests that the evolution of CEMs was linked to partial escape of the ghmC-modified phage genome from cleavage by Cas9 nuclease upon its first exposure to the CRISPR-Cas9 complex following delivery by phage injection. Otherwise, disruption of genome and loss of essential gene function would have destroyed the plaque forming ability even if the cleaved ends were repaired, as was observed with a few high restriction spacers or in infections by unmodified T4(C) mutant phage (55). Consistent with this reasoning, it has been well documented that the ghmC-modified genome is generally resistant to nucleases including the restriction endonucleases (19, 27, 55).

Escape from Cas9 cleavage means phage genome replication would be initiated before the delivered genome is cleaved. Vigorous genome replication, a characteristic of phage life cycle spanning a mere 20-30 minutes, plus the continuing presence of Cas9 then drive evolution and selection of resistant mutations, as per the model described in Results (FIGS. 10A, 10B, and 10C). This must be particularly robust in the case of phage T4 where replication is initiated largely by recombination events (60). The mechanisms are likely complex and not the main focus of this study, but the key implication of our findings is that the timing of CRISPR-Cas9 cleavage relative to the timing of the initiation of phage genome replication is critical for the evolution of the CEMs.

The timescales of CRISPR-Cas cleavage of phage genomes is unknown. A recent report (65) estimates that the association rate of CRISPR-Cas9 complex to a PAM site is ˜40 milliseconds if there were about 5 molecules of Cas9 per E. coli cell. Since the phage T4 genome contains 11,656 PAM sites, it would take about 6 minutes to scan the entire genome. The time taken might be even longer for the ghmC-modified T4 genome than for the unmodified C-genome, although the number of Cas9 molecules per cell is expected to be greater than 5. Therefore, it is safe to assume that it would take a few minutes for CRISPR-Cas9 to find a protospacer sequence in ghmC-genome. By then, many if not most, of the delivered T4 genomes would have initiated replication (62, 66). Consistent with this timeline, our data show that about 10-20% of ghmC-genomes survived Cas9 cleavage and every one of these evolved into a CEM with varying fitness under the continuing pressure of CRISPR-Cas9. Since in nature, this would happen with spacers distributed throughout the phage genome, and in both strands of the genome, the CRISPR system potentially may drive large scale evolution of phage genomes. Some of the mutant phages are expected to be more fit than the parental phage whereas others, probably most as this study indicates, may not have a fitness advantage but would nevertheless remain in the population. Though the specific CRISPR-Cas9 system used here is not native to E. coli, this phenomenon might explain why numerous conservative substitutions in phage genes remain in the closely related phage families even though they may not confer any fitness gain (67, 68). At the same time, all the mutant phages by virtue of their resistance to CRISPR-Cas would be able to contribute to bacterial evolution by horizontal gene transfer and other mechanisms (52).

The timing of CRISPR-Cas cleavage, thus, might provide a critical window for fine-tuning the balance between defense against phages and evolution of phages, and in turn, the bacteria. It could be accomplished by a variety of mechanisms; both phage-based such as the modification of genomes (55, 23, 24,-25), efficiency of initiation of genome replication (69), and inclusion of anti-CRISPR genes (53), and host-based such as the intrinsic catalytic rates of Cas9 cleavage and regulation of cleavage by accessory Cas proteins (70). All of these mechanisms have been described in the literature and it is predicted that some of these slow down the rate of Cas9 cleavage and the progeny phages thus produced likely contain a high frequency of mutations, as has been observed here. The CRISPR-Cas mechanism, thus, might be a part of the global evolutionary system that provides various degrees of advantages to both the bacteria and the phages.

In conclusion, results disclosed herein suggests the possibility that the defensive and counter-defensive systems of the “arms race” between bacteria and phages such as the CRISPR-Cas may have been selected for the survival advantages they provide to both the host and the virus, but not merely to one or the other, such that both the bacteria and the phages may co-exist and co-evolve leading to their dominant presence on Earth.

Although examples show editing phage T4 genome using CRISPR-Cas9 system, it will be appreciated that a CRISPR-Cas9 system may be used to edit genomes of other types of bacteriophages because of the structural and functional similarity of the different types of bacteriophages.

Furthermore, in the present invention, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

### REFERENCES

The following references are referred to above and are incorporated herein by reference:
1. Salmond, G. P., and Fineran, P. C. (2015) A century of the phage: past, present and future, Nat Rev Microbiol 13, 777-786.
2. Rao, V. B., and Black, L. W. (2010) Structure and assembly of bacteriophage T4 head, Virol J 7, 356.
3. Chanishvili, N. (2012) Phage therapy—history from Twort and d'Herelle through Soviet experience to current approaches, Adv Virus Res 83, 3-40.
4. Karam, J. D. (2005) Bacteriophages: the viruses for all seasons of molecular biology, Virol J 2, 19.
5. Wittebole, X., De Roock, S., and Opal, S. M. (2014) A historical overview of bacteriophage therapy as an alternative to antibiotics for the treatment of bacterial pathogens, Virulence 5, 226-235.
6. Lu, T. K., and Koeris, M. S. (2011) The next generation of bacteriophage therapy, Curr Opin Microbiol 14, 524-531.
7. Viertel, T. M., Ritter, K., and Horz, H. P. (2014) Viruses versus bacteria-novel approaches to phage therapy as a tool against multidrug-resistant pathogens, J Antimicrob Chemother 69, 2326-2336.
8. LaFee, S., and Buschman, H. (2017) Novel Phage Therapy Saves Patient with Multidrug-Resistant Bacterial Infection, UC San Diego News Center, San Diego.
9. Bakhshinejad, B., and Sadeghizadeh, M. (2014) Bacteriophages as vehicles for gene delivery into mammalian cells: prospects and problems, Expert Opin Drug Deliv 11, 1561-1574.
10. Pranjol, M. Z., and Hajitou, A. (2015) Bacteriophage-derived vectors for targeted cancer gene therapy, Viruses 7, 268-284.
11. Sunderland, K. S., Yang, M., and Mao, C. (2017) Phage-Enabled Nanomedicine: From Probes to Therapeutics in Precision Medicine, Angew Chem Int Ed Engl 56, 1964-1992.
12. Tao, P., Mahalingam, M., Kirtley, M. L., van Lier, C. J., Sha, J., Yeager, L. A., Chopra, A. K., and Rao, V. B. (2013) Mutated and bacteriophage T4 nanoparticle arrayed F1-V immunogens from Yersinia pestis as next generation plague vaccines, PLoS Pathog 9, e1003495.
13. Tao, P., Mahalingam, M., Marasa, B. S., Zhang, Z., Chopra, A. K., and Rao, V. B. (2013) In vitro and in vivo delivery of genes and proteins using the bacteriophage T4 DNA packaging machine, Proc NatlAcad Sci USA 110, 5846-5851.
14. Tao, P., Mahalingam, M., and Rao, V. B. (2016) Highly Effective Soluble and Bacteriophage T4 Nanoparticle Plague Vaccines Against Yersinia pestis, Methods Mol Biol 1403, 499-518.
15. Ishii, T., and Yanagida, M. (1977) The two dispensable structural proteins (soc and hoc) of the T4 phage capsid; their purification and properties, isolation and characterization of the defective mutants, and their binding with the defective heads in vitro, J Mol Biol 109, 487-514.
16. Li, Q., Shivachandra, S. B., Leppla, S. H., and Rao, V. B. (2006) Bacteriophage T4 capsid: a unique platform for efficient surface assembly of macromolecular complexes, J Mol Biol 363, 577-588.
17. Shivachandra, S. B., Rao, M., Janosi, L., Sathaliyawala, T., Matyas, G. R., Alving, C. R., Leppla, S. H., and Rao, V. B. (2006) In vitro binding of anthrax protective antigen on bacteriophage T4 capsid surface through Hoc-capsid interactions: a strategy for efficient display of large full-length proteins, Virology 345, 190-198.
18. Pires, D. P., Cleto, S., Sillankorva, S., Azeredo, J., and Lu, T. K. (2016) Genetically Engineered Phages: a Review of Advances over the Last Decade, Microbiol Mol Biol Rev 80, 523-543.
19. Karam, J. D., Drake, J. W., Kreuzer, K. N., Mosig, G., Hall, D. H., Eiserling, F. A., Black, L. W., Spicer, E. K., Kutter, E., Carlson, K., and Miller, E. S. (1994) Molecular biology of bacteriophage T4. American Society for Microbiology, American Society for Microbiology, Washington, D.C.
20. Miller, E. S., Kutter, E., Mosig, G., Arisaka, F., Kunisawa, T., and Ruger, W. (2003) Bacteriophage T4 genome, Microbiol Mol Biol Rev 67, 86-156.
21. Mohanraju, P., Makarova, K. S., Zetsche, B., Zhang, F., Koonin, E. V., and van der Oost, J. (2016) Diverse evolutionary roots and mechanistic variations of the CRISPR-Cas systems, Science 353, aad5147.
22. Box, A. M., McGuffie, M. J., O'Hara, B. J., and Seed, K. D. (2015) Functional Analysis of Bacteriophage Immunity through a Type I-E CRISPR-Cas System in Vibrio cholerae and Its Application in Bacteriophage Genome Engineering, J Bacteriol 198, 578-590.
23. Kiro, R., Shitrit, D., and Qimron, U. (2014) Efficient engineering of a bacteriophage genome using the type I-E CRISPR-Cas system, RNA Biol 11, 42-44.
24. Lemay, M. L., Tremblay, D. M., and Moineau, S. (2017) Genome Engineering of Virulent Lactococcal Phages Using CRISPR-Cas9, ACS Synth Biol.
25. Martel, B., and Moineau, S. (2014) CRISPR-Cas: an efficient tool for genome engineering of virulent bacteriophages, Nucleic Acids Res 42, 9504-9513.
26. Pawluk, A., Amrani, N., Zhang, Y., Garcia, B., Hidalgo-Reyes, Y., Lee, J., Edraki, A., Shah, M., Sontheimer, E. J., Maxwell, K. L., and Davidson, A. R. (2016) Naturally Occurring Off-Switches for CRISPR-Cas9, Cell 167, 1829-1838 e1829.
27. Bryson, A. L., Hwang, Y., Sherrill-Mix, S., Wu, G. D., Lewis, J. D., Black, L., Clark, T. A., and Bushman, F. D. (2015) Covalent Modification of Bacteriophage T4 DNA Inhibits CRISPR-Cas9, MBio 6, e00648.
28. Yaung, S. J., Esvelt, K. M., and Church, G. M. (2014) CRISPR/Cas9-mediated phage resistance is not impeded by the DNA modifications of phage T4, PLoS One 9, e98811.
29. Esvelt, K. M., Mali, P., Braff, J. L., Moosburner, M., Yaung, S. J., and Church, G. M. (2013) Orthogonal Cas9 proteins for RNA-guided gene regulation and editing, Nat Methods 10, 1116-1121.
30. Wilson, G. G., Young, K. Y., Edlin, G. J., and Konigsberg, W. (1979) High-frequency generalised transduction by bacteriophage T4, Nature 280, 80-82.
31. Carlson, K., Krabbe, M., Nystrom, A. C., and Kosturko, L. D. (1993) DNA determinants of restriction. Bacteriophage T4 endonuclease II-dependent cleavage of plasmid DNA in vivo, J Biol Chem 268, 8908-8918.
32. Ho, C. K., and Shuman, S. (2002) Bacteriophage T4 RNA ligase 2 (gp24.1) exemplifies a family of RNA ligases found in all phylogenetic domains, Proc Natl Acad Sci USA 99, 12709-12714.
33. Jabbar, M. A., and Snyder, L. (1984) Genetic and physiological studies of an Escherichia coli locus that restricts polynucleotide kinase- and RNA ligase-deficient mutants of bacteriophage T4, J Virol 51, 522-529.
34. Amitsur, M., Morad, I., and Kaufmann, G. (1989) In vitro reconstitution of anticodon nuclease from components encoded by phage T4 and Escherichia coli CTr5X, EMBO J 8, 2411-2415.
35. Arisaka, F., Yap, M. L., Kanamaru, S., and Rossmann, M. G. (2016) Molecular assembly and structure of the bacteriophage T4 tail, Biophys Rev 8, 385-396.
36. Black, L. W., and Rao, V. B. (2012) Structure, assembly, and DNA packaging of the bacteriophage T4 head, Adv Virus Res 82, 119-153.
37. Rao, V. B., and Feiss, M. (2008) The bacteriophage DNA packaging motor, Annu Rev Genet 42, 647-681.
38. Sun, S., Kondabagil, K., Draper, B., Alam, T. I., Bowman, V. D., Zhang, Z., Hegde, S., okine, A., Rossmann, M. G., and Rao, V. B. (2008) The structure of the phage T4 DNA packaging motor suggests a mechanism dependent on electrostatic forces, Cell 135, 1251-1262.
39. Sun, S., Rao, V. B., and Rossmann, M. G. (2010) Genome packaging in viruses, Curr Opin Struct Biol 20, 114-120.
40. Yap, M. L., and Rossmann, M. G. (2014) Structure and function of bacteriophage T4, Future Microbiol 9, 1319-1327.
41. Zhang, Z., Kottadiel, V. I., Vafabakhsh, R., Dai, L., Chemla, Y. R., Ha, T., and Rao, V. B. (2011) A promiscuous DNA packaging machine from bacteriophage T4, PLoS biology 9, e1000592.
42. Mosig, G., Gewin, J., Luder, A., Colowick, N., and Vo, D. (2001) Two recombination-dependent DNA replication pathways of bacteriophage T4, and their roles in mutagenesis and horizontal gene transfer. Proc. Natl. Acad. Sci. U.S.A 98, 8306-8311.
43. Kleinstiver, B. P., Prew, M. S., Tsai, S. Q., Topkar, V. V., Nguyen, N. T., Zheng, Z., Gonzales, A. P., Li, Z., Peterson, R. T., Yeh, J. R., Aryee, M. J., and Joung, J. K. (2015) Engineered CRISPR-Cas9 nucleases with altered PAM specificities, Nature 523, 481-485.
44. Selick, H. E., Kreuzer, K. N., and Alberts, B. M. (1988) The bacteriophage T4 insertion/substitution vector system. A method for introducing site-specific mutations into the virus chromosome. J. Biol. Chem. 263, 11336-11347.
45. Runnels, J. M., Soltis, D., Hey, T., and Snyder, L. (1982) Genetic and physiological studies of the role of the RNA ligase of bacteriophage T4, J Mol Biol 154, 273-286.
46. Tao, P., Li, Q., Shivachandra, S. B., and Rao, V. B. (2017) Bacteriophage T4 as a Nanoparticle Platform to Display and Deliver Pathogen Antigens: Construction of an Effective Anthrax Vaccine, Methods Mol Biol 1581, 255-267.
47. R. W. Hendrix, Bacteriophage genomics. Curr Opin Microbiol 6, 506-511 (2003).
48. K. D. Seed, Battling Phages: How Bacteria Defend against Viral Attack. PLoS Pathog 11, e1004847 (2015).
49. B. Wiedenheft, S. H. Sternberg, J. A. Doudna, RNA-guided genetic silencing systems in bacteria and archaea. Nature 482, 331-338 (2012).
50. P. Mali, K. M. Esvelt, G. M. Church, Cas9 as a versatile tool for engineering biology. Nat Methods 10, 957-963 (2013).
51. D. Paez-Espino et al., CRISPR immunity drives rapid phage genome evolution in Streptococcus thermophilus. MBio 6, (2015).
52. B. Koskella, M. A. Brockhurst, Bacteria-phage coevolution as a driver of ecological and evolutionary processes in microbial communities. FEMS Microbiol Rev 38, 916-931 (2014).
53. J. Bondy-Denomy, A. Pawluk, K. L. Maxwell, A. R. Davidson, Bacteriophage genes that inactivate the CRISPR/Cas bacterial immune system. Nature 493, 429-432 (2013).
54. A. Pawluk et al., Inactivation of CRISPR-Cas systems by anti-CRISPR proteins in diverse bacterial species. Nat Microbiol 1, 16085 (2016).
55. P. Tao, X. Wu, W. C. Tang, J. Zhu, V. Rao, Engineering of Bacteriophage T4 Genome Using CRISPR-Cas9. ACS Synth Biol 6, 1952-1961 (2017).
56. M. E. Santos, J. W. Drake, Rates of spontaneous mutation in bacteriophage T4 are independent of host fidelity determinants. Genetics 138, 553-564 (1994).
57. J. W. Drake, A constant rate of spontaneous mutation in DNA-based microbes. Proc Natl Acad Sci USA 88, 7160-7164 (1991).
58. K. Carlson, A. Overvatn, Bacteriophage T4 endonucleases II and IV, oppositely affected by dCMP hydroxymethylase activity, have different roles in the degradation and in the RNA polymerase-dependent replication of T4 cytosine-containing DNA. Genetics 114, 669-685 (1986).
59. G. Mosig, Recombination and recombination-dependent DNA replication in bacteriophage T4. Annu Rev Genet 32, 379-413 (1998).
60. K. N. Kreuzer, J. R. Brister, Initiation of bacteriophage T4 DNA replication and replication fork dynamics: a review in the Virology Journal series on bacteriophage T4 and its relatives. Virol J 7, 358 (2010).
61. J. W. George, B. A. Stohr, D. J. Tomso, K. N. Kreuzer, The tight linkage between DNA replication and double-strand break repair in bacteriophage T4. Proc Natl Acad Sci USA 98, 8290-8297 (2001).
62. A. W. Kozinski, Origins of T4 DNA replication Bacteriophage T4 C. K. Mathews, E. M. Kutter, G. Mosig, P. B. Berget, Eds., (America Society for Microbiology, Washington, D C, 1983).
63. L. Sun et al., Cryo-EM structure of the bacteriophage T4 portal protein assembly at near-atomic resolution. Nat Commun 6, 7548 (2015).
64. Z. Chen et al., Cryo-EM structure of the bacteriophage T4 isometric head at 3.3-A resolution and its relevance to the assembly of icosahedral viruses. Proc Natl Acad Sci USA 114, E8184-E8193 (2017).
65. D. L. Jones et al., Kinetics of dCas9 target search in Escherichia coli. Science 357, 1420-1424 (2017).
66. G. Mosig, Relationship of T4 DNA replication and recombination. C. K. Mathews, E. M. Kutter, G. Mosig, P. B. Berget, Eds., (America Society for Microbiology, Washington, D C, 1983).
67. V. M. Petrov, S. Ratnayaka, J. M. Nolan, E. S. Miller, J. D. Karam, Genomes of the T4-related bacteriophages as windows on microbial genome evolution. Virol J 7, 292 (2010).
68. J. M. Nolan, V. Petrov, C. Bertrand, H. M. Krisch, J. D. Karam, Genetic diversity among five T4-like bacteriophages. Virol J 3, 30 (2006).
69. I. J. Molineux, D. Panja, Popping the cork: mechanisms of phage genome ejection. Nat Rev Microbiol 11, 194-204 (2013).
70. A. V. Wright et al., Rational design of a split-Cas9 enzyme complex. Proc Natl Acad Sci USA 112, 2984-2989 (2015).
71. G.P.C. Salmond, P. C. Fineran, A century of the phage: past, present and future. Nature Reviews Microbiology 13, 777-786 (2015).

All documents, patents, journal articles and other materials cited in the present application are incorporated herein by reference.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An engineered system for editing a bacteriophage genome comprising:
a bacterial host cell adapted to produce an engineered bacteriophage using a Clustered Regularly Interspaced Short Palindromic Repeat (CRISPR)-CRISPR associated protein (Cas) (CRISPR-Cas), the bacterial host cell comprising:
a first nucleic acid sequence encoding a Cas protein, and
a second nucleic acid sequence encoding a guide RNA (gRNA) comprising a trans-activating crRNA (tracrRNA) and a guide sequence complementary to a target DNA sequence in a bacteriophage genome;
the first nucleic acid sequence and the second nucleic acid sequence being operably linked to a same regulatory element or different regulatory elements operable in the bacterial host cell, on same or different vectors, whereby the Cas9 protein and the at least one gRNA being expressed and forming a CRISPR-Cas complex in the bacterial host cell, wherein the Cas protein and the gRNA are engineered to occur together.

2. The engineered system of claim 1, wherein the first nucleic acid sequence encoding the Cas protein and the second nucleic acid sequence encoding the guide RNA (gRNA) are located in a same CRISPR-Cas spacer plasmid and are operably linked to a same regulatory element operable in the bacterial host cell.

3. The engineered system of claim 2, wherein the Cas protein and the guide sequence are constitutively expressed under a control of the promoter.

4. The engineered system of claim 1, wherein the Cas protein is type II CRISPR-associated nuclease enzyme Cas9 derived from Streptococcus pyogenes.

5. The engineered system of claim 1, wherein the bacteriophage is bacteriophage T4.

6. The engineered system of claim 1, wherein the target DNA sequence is a protospacer immediately preceding a protospacer adjacent motif (PAM) in a gene of the bacteriophage.

7. The engineered system of claim 1, wherein a nucleic acid sequence encoding the guide sequence comprises a spacer of about 20 nucleotides (nt) from a gene encoding bacteriophage capsid protein.

8. The engineered system of claim 7, wherein the nucleic acid sequence encoding the guide sequence comprises a sequence set forth in SEQ ID NO: 1 or 19.

9. The engineered system of claim 1, wherein a nucleic acid sequence encoding the guide sequence comprises a spacer of about 20 nucleotides (nt) from a gene encoding a bacteriophage portal protein.

10. The engineered system of claim 9, wherein the nucleic acid sequence encoding the guide sequence comprises a sequence set forth in SEQ ID NO: 2 or 13.

11. The engineered system of claim 1, wherein the bacterial host cell further contains a DNA repair template comprising a donor DNA sequence flanked by a left homologous arm and a right homologous arm, the donor DNA sequence comprising a mutation to the bacteriophage genome, the left and right homologous arms being sufficient long to allow the donor DNA sequence being introduced into the bacteriophage genome by homologous recombination.

12. The engineered system of claim 11, wherein the left homologous arm and the right homologous arm have a length of about 50 bp to about 1.2 kb.

13. The engineered system of claim 11, wherein the DNA repair template is included in a donor plasmid.

14. The engineered system of claim 1, wherein the bacterial host cell further contains a genome of a bacteriophage including the target DNA sequence.

15. The engineered system of claim 14, wherein the bacteriophage is a glucosylhydroxymethyl cytosine (ghmC)-unmodified mutant phage.

16. The engineered system of claim 15, wherein the ghmC-unmodified mutant phage contains an amber mutation in gene 42 that codes for deoxycytidine monophosphate hydroxymethylase (g42) and an amber mutation in gene 56 that codes for deoxycytidine triphosphatase (dCTPase).

17. The engineered system of claim 1, wherein the bacterial host cell is Escherichia coli (E. coli) bacteria.

18. An engineered system for editing a bacteriophage genome comprising:
a bacterial host cell adapted to produce an engineered bacteriophage using CRISPR-Cas comprising:
a first nucleic acid sequence encoding a Cas9 protein, and
at least one nucleic acid sequence encoding at least one guide RNA (gRNA) comprising a trans-activating crRNA (tracrRNA) and two or more guide sequences respectively complementary to two or more target DNA sequences in a bacteriophage genome;
the first nucleic acid sequence and the at least one nucleic acid sequence encoding the at least one guide RNA being operably linked to a same or different regulatory elements operable in the bacterial host cell, on same or different vectors, such that the Cas9 protein and the at least one gRNA are expressed and form at least one CRISPR-Cas complex in the bacterial host cell, wherein the Cas protein and the at least one gRNA are engineered to occur together.

19. The engineered system of claim 18, wherein the first nucleic acid sequence encoding the Cas protein and the at least one nucleic acid sequence encoding at least one guide RNA are located in a same CRISPR-Cas spacer plasmid and are operably linked to a same regulatory element operable in the bacterial host cell.

20. The engineered system of claim 18, wherein the Cas protein and the at least one guide RNA are constitutively expressed.

21. The engineered system of claim 18, wherein each of the one or more target DNA sequences is a protospacer immediately preceding a protospacer adjacent motif (PAM) in a gene of the bacteriophage.

22. The engineered system of claim 18, wherein the bacteriophage is bacteriophage T4.

23. The engineered system of claim 18, wherein the bacteriophage is a glucosylhydroxymethyl cytosine (ghmC)-unmodified mutant phage.

24. The engineered system of claim 18, wherein a nucleic acid sequence encoding the two or more guide sequences comprises a spacer of about 20 nucleotides (nt) from a gene encoding bacteriophage capsid protein.

25. The engineered system of claim 18, wherein a nucleic acid sequence encoding the two or more guide sequences comprises a spacer of about 20 nucleotides (nt) from a gene encoding a bacteriophage portal protein.

26. The engineered system of claim 18, wherein the bacterial host cell further contains a DNA repair template comprising a donor DNA sequence flanked by a left homologous arm and a right homologous arm, the donor DNA sequence comprising a mutation to the bacteriophage genome, the left and right homologous arms being sufficient long to allow the donor DNA sequence being inserted into the bacteriophage genome by homologous recombination.

27. The engineered system of claim 26, wherein the DNA repair template is included in a donor plasmid.

28. The engineered system of claim 27, wherein the bacterial host cell contains a genomic DNA of the bacteriophage that includes the one or more target DNA sequences.

29. The engineered system of claim 18, wherein the at least one guide RNA comprises two guide sequences including a first guide sequence being complementary to a first target DNA sequence and a second guide sequence being complementary to a second target DNA sequence, and the first target DNA sequence and the second target DNA sequence are two adjacent protospacers immediately preceding two respective PAM sequences in a bacteriophage gene.

30. The engineered system of claim 29, wherein the bacterial host cell further comprises a genome of the bacteriophage that includes the two adjacent protospacers immediately preceding the two respective PAM sequences.

31. A kit for editing a bacteriophage genome comprising:
one or more vectors containing:
a first nucleic acid sequence encoding a Cas9 protein, and
at least one nucleic acid sequence encoding at least one guide RNA (gRNA) comprising a trans-activating crRNA (tracrRNA) and one or more guide sequences respectively complementary to one or more target DNA sequences in a bacteriophage genome;
the first nucleic acid sequence and the at least one nucleic acid sequence encoding the at least one guide RNA being operably linked to a same regulatory element or different regulatory elements operable in a bacterial host cell, whereby allowing the Cas9 protein and the at least one gRNA to be expressed in the bacterial host cell, wherein the Cas protein and the at least one gRNA are engineered to occur together.

32. The kit of claim 31, wherein the first nucleic acid sequence encoding the Cas protein and the at least one nucleic acid sequence encoding the at least one guide RNA (gRNA) are located in different vectors.

33. The kit of claim 31, wherein the first nucleic acid sequence encoding the Cas protein and the at least one nucleic acid sequence encoding the at least one guide RNA (gRNA) are located in a same CRISPR-Cas spacer vector and operably linked to a same regulatory element operable in the bacterial host cell.

34. The kit of claim 31, wherein the CRISPR-Cas spacer vector is a plasmid, and the Cas9 protein and the at least one guide RNA are constitutively expressed.

35. The kit of claim 31, wherein each of the one or more target DNA sequences is a protospacer immediately preceding a protospacer adjacent motif (PAM) in a gene of the bacteriophage.

36. The kit claim 31, wherein a nucleic acid sequence encoding the one or more guide sequences comprises a spacer of about 20 nucleotides (nt) from a gene encoding bacteriophage capsid protein.

37. The kit claim 31, wherein a nucleic acid sequence encoding the one or more guide sequences comprises a spacer of about 20 nucleotides (nt) from a gene encoding a bacteriophage portal protein.

38. The kit of claim 31, wherein a nucleotide sequence encoding the one or more guide sequences comprises a sequence set forth in SEQ ID NO: 1 or 19.

39. The kit of claim 31, wherein a nucleotide sequence encoding the one or more guide sequences comprises a sequence set forth in SEQ ID NO: 2 or 13.

40. The kit of claim 31, wherein the at least one guide RNA comprises two guide sequences including a first guide sequence being complementary to a first target DNA sequence and a second guide sequence being complementary to a second target DNA sequence, and the first target DNA sequence and the second target DNA sequence are two adjacent protospacers immediately preceding two respective PAM sequences in a bacteriophage gene.

41. The kit of claim 31, further comprising a DNA repair template including a donor DNA sequence flanked by a left homologous arm and a right homologous arm, and the donor DNA sequence comprising a mutation to the bacteriophage genome, the left and right homologous arms being sufficient long to allow the donor DNA sequence being introduced into the bacteriophage genome by homologous recombination.

42. The kit of claim 41, wherein the DNA repair template is included in a donor plasmid.

43. The kit of claim 31, further comprising a glucosylhydroxymethyl cytosine (ghmC)-unmodified mutant bacteriophage.

44. A method comprising:
introducing a bacteriophage into a bacterial host cell containing a CRISPR-Cas spacer vector and a DNA repair template;
wherein the CRISPR-Cas spacer vector comprises:
a first nucleic acid sequence encoding a Cas9 protein, and
at least one nucleic acid sequence encoding at least one guide RNA (gRNA) comprising a trans-activating crRNA (tracrRNA) and one or more guide sequences respectively complementary to one or more target DNA sequences in a bacteriophage genome;
wherein the first nucleic acid sequence and the at least one nucleic acid sequence encoding the at least one guide RNA are operably linked to a regulatory element operable in the bacterial host cell, whereby the Cas9 protein and the at least one gRNA are expressed and form at least one CRISPR-Cas complex in the bacterial host cell, wherein the Cas protein and the at least one gRNA are engineered to occur together.
wherein the at least one gRNA targets the one or more target DNA sequences in the bacteriophage genome and the Cas9 protein cleaves the bacteriophage genome, thereby generating one or more double-strand breaks in the one or more target DNA sequences; and
wherein the DNA repair template includes a donor DNA sequence flanked by DNA segments homologous to end sequences of one of the one or more double-strand breaks, and the donor DNA sequence includes at least one mutation to the bacteriophage genome, whereby altering the bacteriophage genome after the donor DNA sequence being inserted into one of the one or more double-strand breaks through homology directed repair.

45. The method of claim 44, wherein the DNA repair template is included in a Cas9-resistant donor plasmid.

46. The method of claim 44, wherein the CRISPR-Cas spacer vector is a plasmid, and the Cas9 protein and the at least one guide RNA are constitutively expressed in the bacterial host cell.

47. The method of claim 44, wherein each of the one or more target DNA sequences is a protospacer immediately preceding a protospacer adjacent motif (PAM) in a gene of the bacteriophage.

48. The method of claim 44, wherein the bacteriophage is bacteriophage T4.

49. The method of claim 44, wherein the bacteriophage is a glucosylhydroxymethyl cytosine (ghmC)-unmodified mutant phage.

50. The method of claim 44, wherein a nucleic acid sequence encoding the one or more guide sequences comprises a spacer of about 20 nucleotides (nt) from a gene encoding bacteriophage capsid protein.

51. The method of claim 50, wherein the nucleic acid sequence encoding the one or more guide sequences comprises a sequence set forth in SEQ ID NO: 1 or 19.

52. The method of claim 44, wherein a nucleic acid sequence encoding the one or more guide sequences comprises a spacer of about 20 nucleotides (nt) from a gene encoding a bacteriophage portal protein.

53. The method of claim 52, wherein the nucleic acid sequence encoding the one or more guide sequences comprises a sequence set forth in SEQ ID NO: 2 or 13.

54. The method of claim 44, wherein the DNA segments homologous to end sequences of one of the one or more double-strand breaks are sufficient long to allow the donor DNA sequence being introduced into the bacteriophage genome by homologous recombination.

55. The method of claim 44, wherein the DNA segments homologous to end sequences of one of the one or more double-strand breaks have a length of about 50 bp to about 1.2 kb.

56. The method of claim 44,
wherein the at least one guide RNA comprises two guide sequences including a first guide sequence being complementary to a first target DNA sequence and a second guide sequence being complementary to a second target DNA sequence, and the first target DNA sequence and the second target DNA sequence being two adjacent protospacers immediately preceding two respective PAM sequences in a bacteriophage gene;
wherein the Cas9 protein cleaves the bacteriophage gene at two adjacent sites in the two adjacent protospacers under a direction of the at least one guide RNA, thereby creating a double-strand break in the bacteriophage genome with an intervening sequence between the two adjacent sites being excised; and
wherein the DNA repair template includes a donor DNA sequence flanked by DNA segments homologous to end sequences of the double-strand break, allowing the excised intervening sequence being replaced by the donor DNA sequence.

57. The method of claim 44, further comprising selecting a spacer of about 20 nucleotides (nt) from a genomic DNA sequence of the bacteriophage for encoding the one or more guide sequences.

58. The method of claim 44, further comprising co-delivering into the bacterial host cell the CRISPR-Cas spacer vector and the DNA repair template.

59. A method of determining an essentiality of a target gene of a bacteriophage comprising:
introducing a null mutation to a target gene of a bacteriophage genome by the method of claim 44 using a DNA repair template comprising the null mutation, causing the target gene to fail to be translated into a function protein product; and
performing a plaque assay for infection of bacterial host cells with bacteriophage having the null mutation and with wild type bacteriophage respectively;
wherein target gene is determined to be nonessential if plaque formation for infection of bacterial host cells with bacteriophage that has the null mutation is similar to plaque formation for infection of bacterial host cells with wild type bacteriophage.

60. The method of claim 59, wherein the null mutation is an amber mutation.

61. The method of claim 59, wherein the null mutation includes a deletion of at least a portion of the target gene, the null mutation being introduced into the genome of the bacteriophage by the method of claim 56.
